(19) United States
(12) Patent Application Publication
(10) Pub. No.: US 2026/0043082 A1
(43) Pub. Date: Feb. 12, 2026

(54) PREDICTING PATIENT RESPONSE

(71) Applicant: ONCOHOST LTD., Binyamina (IL)

(72) Inventors: Coren LAHAV, Tel Aviv (IL); Itamar SELA, Ramat-Gan (IL); Yehonatan ELON, Jerusalem (IL); Michal HAREL, Hod Hasharon (IL); Eyal JACOB, Haifa (IL); Ben YELLIN, Ganei Am (IL)

(21) Appl. No.: 19/102,780

(22) PCT Filed: Aug. 10, 2023
(86) PCT No.: PCT/IL2023/050841

§ 371 Date: Feb. 10, 2025

Related U.S. Application Data
(63) Continuation-in-part of application No. PCT/IL2022/050881, filed on Aug. 11, 2022.
(60) Provisional application No. 63/423,551, filed on Nov. 8, 2022.
(60) Provisional application No. 63/442,174, filed on Jan. 31, 2023.
(60) Provisional application No. 63/465,026, filed on May 9, 2023.

(30) Foreign Application Priority Data
Aug. 11, 2022 (WO) PCT/IL2022/050881

(51) Int. Cl. C12Q 1/6876; G16H 20/10

(52) U.S. Cl. CPC C12Q 1/6876; G16H 20/10; C12Q 2600/106; C12Q 2600/158

(57) ABSTRACT

Methods of predicting response of a subject suffering from cancer to an anti-PD-1/L1 immunotherapy, as a monotherapy or combination therapy, comprising calculating a resistance score for factors expressed by the subject, summing the resistance score to produce a total resistance score, wherein a total resistance score beyond a predetermined threshold indicates a subject is predicted to be resistant to the anti-PD-1/L1 immunotherapy as a monotherapy or combination therapy, are provided.

### CROSS REFERENCE TO RELATED APPLICATIONS

This application claims the benefit of priority of International Patent Application No. PCT/IL2022/050881 filed on Aug. 11, 2022, U.S. Provisional Patent Application No. 63/423,551 filed on Nov. 8, 2022, U.S. Provisional Patent Application No. 63/442,174 filed on Jan. 31, 2023, and U.S. Provisional Patent Application No. 63/465,026 filed on May 9, 2023, the contents of which are all incorporated herein by reference in their entirety.

### FIELD OF THE INVENTION

The present invention is in the field of patient-specific diagnostics.

### BACKGROUND OF THE INVENTION

Immunotherapy based on immune checkpoint inhibitors (ICIs) represents a significant breakthrough in clinical oncology. ICIs augment an anti-tumor immune response by targeting checkpoint proteins such as PD-1, PD-L1 and CTLA-4 expressed on tumor and immune cells. Although ICI therapies can achieve unprecedented long-term disease control across multiple tumor types, efficacy varies widely between patients, with the majority exhibiting primary or subsequent acquired resistance to therapy. In metastatic non-small cell lung cancer (NSCLC) for which ICI regimens are standard of care, response rates range between 10%-50% depending on tumor PD-L1 expression as well as type and line of treatment. Identifying patients who are likely to benefit from ICI therapy is still a major clinical challenge because available predictive biomarkers are not sufficiently accurate.

To date, tumor PD-L1 expression and tumor mutational burden (TMB) are the most prominent biomarkers for predicting ICI response. While immunohistochemical tests for assessing PD-L1 expression in tumor tissue are used as companion diagnostics for informing treatment decisions in NSCLC, TMB is still not used routinely. According to current guidelines for NSCLC patients lacking oncogenic driver mutations, patients with high tumor PD-L1 expression (defined as PD-L1 expression on at least 50% of tumor cells) are eligible for first-line ICI monotherapy whereas ICI in combination with chemotherapy is the preferred choice for patients with PD-L1 expression <50%. However, clinical evidence demonstrates limitations of the PD-L1 biomarker in predicting ICI response. For example, in the KEYNOTE-024 trial, approximately half of the PD-L1-high patient cohort did not respond to pembrolizumab monotherapy. Moreover, several clinical trials report clinical benefit from ICI therapies in some patients with low tumor PD-L1 expression. Notably, although both PD-L1 and TMB are related to the mechanism of action of ICIs, these biomarkers do not account for the complexity of tumor-immune system interactions and the heterogenous mechanisms underpinning response and resistance to ICI therapy. In addition, the PD-L1 test requires tumor tissues, which are sometimes not available.

To address this, a more comprehensive characterization of the tumor, the tumor microenvironment (TME), peripheral immune cells and other host factors is needed. Indeed, a growing number of emerging predictive biomarkers for ICI outcome are based on tumor genomic features and expression patterns, the abundance and phenotype of tumor-infiltrating lymphocytes in the TME, peripheral T cell dynamics, and properties of other immune cell types. Importantly, integrative models combining several biomarkers show promise for improving predictive performance, presumably by better capturing the multi-faceted nature of therapeutic benefit. For example, combining the PD-L1 and TMB biomarkers improves prediction of response to ICI therapy in lung cancer patients. In addition, several studies have demonstrated improved prediction of ICI outcomes using integrated genomic, transcriptomic, and immune repertoire data. Although such models are promising, they are limited in that they are based on multiple assays and usually require tumor tissue specimens.

Plasma proteomics represents a promising strategy for predictive biomarker discovery. Circulating blood contains thousands of proteins derived from the developing tumor, TME, peripheral immune cells and other host cells. As such, the plasma proteome reflects tumor-intrinsic properties, immune cell dynamics, angiogenesis, extracellular matrix remodeling and metabolic changes, making it a rich source of potential biomarkers that can be sampled in a minimally invasive manner and measured with a single assay. A method of determining patient-specific response to immunotherapy that integrates PD-L1 levels and plasma proteomic data, is greatly needed.

### SUMMARY OF THE INVENTION

The present invention provides methods of predicting response of a subject suffering from a PD-L1 high, low, or negative cancer to a monotherapy or combination therapy, comprising calculating a resistance score for factors expressed by the subject, combining the resistance score to produce a total resistance score, wherein a total resistance score beyond a predetermined threshold indicates a subject is predicted to be resistant to the monotherapy or combination therapy.

According to a first aspect, there is provided a method of predicting response of a subject suffering from a PD-L1 high cancer to a monotherapy comprising an anti-PD-1/PD-L1 immunotherapy, the method comprising:
a. receiving factor expression levels for a plurality of factors
   i. in a population of subjects suffering from cancer and known to respond to the monotherapy (responders);
   ii. in a population of subjects suffering from cancer and known to not respond to the monotherapy (non-responders); and
   iii. in the subject;
b. calculate for factors of the plurality of factors a resistance score, wherein the calculating comprises applying a machine learning algorithm trained on a training set comprising the received factor expression levels in responders and non-responders and the sex of each of the responders and non-responders to individual received factor expression levels from the subject and the subject's sex and wherein the machine learning algorithm outputs the resistance score; and
c. combine the calculated resistance scores to produce a total resistance score;
wherein a subject with a total resistance score beyond a predetermined threshold is predicted to not respond to the monotherapy and a subject with a total resistance score within the predetermined threshold is predicted to respond to the monotherapy;
   thereby predicting response of a subject to a monotherapy.

According to some embodiments, the total resistance score is converted to a total response score by the equation (1-total resistance score) and wherein a total response score above a predetermined threshold indicates the subject is responsive to the monotherapy and a total response score below a predetermined threshold indicates the subject is not responsive to the monotherapy.

According to some embodiments, the total resistance score is converted to a total response score by the equation (10-total resistance score) and wherein a total response score above a predetermined threshold indicates the subject is responsive to the monotherapy and a total response score below a predetermined threshold indicates the subject is not responsive to the monotherapy.

According to some embodiments, the training set further comprises received factor expression levels in subjects suffering from cancer and known to respond to a combination therapy comprising an anti-PD-1/PD-L1 immunotherapy and chemotherapy (combo-responders) and received factor expression levels in subject suffering from cancer and known to not respond to the combination therapy (combo-non-responders) and the sex of each of the combo-responders and combo-non-responders.

According to another aspect, there is provided a method of predicting response of a subject suffering from a PD-L1 low or negative cancer to a combination therapy comprising an anti-PD-1/PD-L1 immunotherapy and chemotherapy, the method comprising:
a. receiving factor expression levels for a plurality of factors
   i. in a population of subjects suffering from cancer and known to respond to the combination therapy (responders);
   ii. in a population of subjects suffering from the cancer and known to not respond to the combined therapy (non-responders); and
   iii. in the subject;
b. calculate for factors of the plurality of factors a resistance score, wherein the calculating comprises applying a machine learning algorithm trained on a training set comprising the received factor expression levels in responders and non-responders and the sex of each of the responders and non-responders to individual received factor expression levels from the subject and the subject's sex and wherein the machine learning algorithm outputs the resistance score; and
c. combine the calculated resistance scores to produce a total resistance score;
wherein a subject with a total resistance score beyond a predetermined threshold is predicted to not respond to the combination therapy and a subject with a total resistance score within the predetermined threshold is predicted to respond to the combination therapy; and
   thereby predicting response of a subject to a combination therapy.

According to some embodiments, the total resistance score is converted to a total response score by the equation (1-total resistance score) and wherein a total response score above a predetermined threshold indicates the subject is responsive to the combination therapy and a total response score below a predetermined threshold indicates the subject is not responsive to the combination therapy.

According to some embodiments, the total resistance score is converted to a total response score by the equation (10-total resistance score) and wherein a total response score above a predetermined threshold indicates the subject is responsive to the combination therapy and a total response score below a predetermined threshold indicates the subject is not responsive to the combination therapy.

According to another aspect, there is provided a method of predicting response of a subject suffering from cancer to an anti-PD-1/PD-L1 immunotherapy, the method comprising:
a. receiving factor expression levels for a plurality of factors
   i. in a population of subjects suffering from cancer and known to respond to the immunotherapy (responders);
   ii. in a population of subjects suffering from cancer and known to not respond to the immunotherapy (non-responders); and
   iii. in the subject;
b. calculate for factors of the plurality of factors a resistance score, wherein the calculating comprises applying a machine learning algorithm trained on a training set comprising the received factor expression levels in responders and non-responders and the sex of each of the responders and non-responders, to individual received factor expression levels from the subject and the subject's sex and wherein the machine learning algorithm outputs the resistance score; and
c. combine the calculated resistance scores to produce a total resistance score;
wherein a subject with a total resistance score beyond a predetermined threshold is predicted to not respond to the anti-PD-1/PD-L1 immunotherapy;
   thereby predicting response of a subject to an anti-PD-1/PD-L1 immunotherapy.

According to some embodiments, the training set further comprises received factor expression levels in subjects suffering from cancer and known to respond to a monotherapy comprising an anti-PD-1/PD-L1 immunotherapy (mono-responders) and received factor expression levels in subjects suffering from cancer and known to not respond to the monotherapy (mono-non-responders) and the sex of each of the mono-responders and mono-non-responders.

According to some embodiments, the total resistance score is converted to a total response score by the equation (1-total resistance score) and wherein a total response score above a predetermined threshold indicates the subject is responsive to the immunotherapy and a total response score below a predetermined threshold indicates the subject is not responsive to the immunotherapy.

According to some embodiments, the total resistance score is converted to a total response score by the equation (10-total resistance score) and wherein a total response score above a predetermined threshold indicates the subject is responsive to the immunotherapy and a total response score below a predetermined threshold indicates the subject is not responsive to the immunotherapy.

According to some embodiments, the plurality of factors comprises at least two factors selected from the factors provided in Table 4.

According to some embodiments, the plurality of factors consists of factors selected from Table 4.

According to some embodiments, the responders and non-responders are determined based on progression free survival (PFS) at 1 year after initiation of the monotherapy or combination therapy.

According to some embodiments, the method comprises before (b) selecting a subset of the plurality of factors, wherein the subset comprises factors that best differentiate between the responders and non-responders, and wherein the calculating is for each factor of the subset.

According to some embodiments, the selecting comprises applying a statistical test to the received factor expression levels, optionally wherein the statistical test is a Kolmogorov-Smirnov test.

According to some embodiments, the subset consists of at least 50 factors.

According to some embodiments, the factor expression level is from a time point before administration of an anti-PD-1/PD-L1 immunotherapy to the subject.

According to some embodiments, the combining is averaging.

According to some embodiments, the combining comprises determining the total number of factors with a resistance score above a predetermined threshold and producing a total resistance score proportional to the total number.

According to some embodiments, the method further comprises performing a dimensionality reduction step with respect to the plurality of factors, to reduce the number of factors in the plurality.

According to some embodiments, the cancer is selected from hepato-biliary cancer, cervical cancer, urogenital cancer, anogenital, testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer, renal cancer, skin cancer, head and neck cancer, leukemia and lymphoma.

According to some embodiments, the cancer is selected from lung cancer, skin cancer, anogenital cancer, cervical cancer and head and neck cancer.

According to some embodiments, the cancer is non-small cell lung cancer (NSCLC).

According to some embodiments, the cancer is a tyrosine kinase inhibitor resistant cancer.

According to some embodiments, the predetermined threshold is determined by performing a cross-validation within the training set or is the median score of the training set.

According to some embodiments, the plurality of factors is at least 200 factors.

According to some embodiments, the factors expression levels are factors expression levels in a biological sample provided by the subjects.

According to some embodiments, the biological sample is selected from blood plasma, whole blood, blood serum or peripheral blood mononuclear cells.

According to some embodiments, the biological sample is blood plasma or blood serum.

According to some embodiments, the method further comprises administering the monotherapy to the subject predicted to respond to the monotherapy or administering a combined therapy comprising the anti-PD-1/PD-L1 immunotherapy and chemotherapy to the subject predicted to not respond to the monotherapy.

According to some embodiments, the method further comprises administering the combination therapy to the subject predicted to respond to the combination therapy or administering an alternative therapy to the subject predicted to not respond to the combination therapy.

According to some embodiments, the method further comprises administering said anti-PD-1/PD-L1 immunotherapy to said subject predicted to respond to said anti-PD-1/PD-L1 immunotherapy or administering an alternative therapy to said subject predicted to not respond to said anti-PD-1/PD-L1 immunotherapy.

According to some embodiments, the anti-PD-1/PD-L1 immunotherapy is selected from Pembrolizumab, Nivolumab, Durvalumab and Atezolizumab.

According to some embodiments, the chemotherapy is selected from Carboplatin, Paclitaxel, Nab-Paclitaxel, Pemetrexed, Vinorelbine, and Cisplatin.

According to some embodiments, the combination therapy is selected from:
a. Carboplatin, Durvalumab, and Paclitaxel;
b. Atezolizumab, Bevacizumab, Carboplatin, and Paclitaxel;
c. Carboplatin, Nab-Paclitaxel, and Pembrolizumab;
d. Carboplatin, Nivolumab, and Paclitaxel;
e. Carboplatin, Nivolumab, Pemetrexed;
f. Carboplatin, Paclitaxel, Pembrolizumab;
g. Carboplatin, Paclitaxel, Pembrolizumab, and radiation;
h. Carboplatin, and Pembrolizumab;
i. Carboplatin, Pembrolizumab, and Pemetrexed;
j. Carboplatin, Pembrolizumab, and Vinorelbine; and
k. Cisplatin, Pembrolizumab, and Pemetrexed.

According to some embodiments, predicting response comprises predicting overall survival.

According to some embodiments, predicting response comprises predicting progression free survival.

According to some embodiments, progression free survival is at 1 year after initiation of the monotherapy or combination therapy.

According to some embodiments, progression free survival is at 1 year after initiation of the immunotherapy.

According to some embodiments, the subject suffers from a negative PD-L1 cancer.

According to some embodiments, PD-L1 high cancer comprises at least 50% of cancer cells being positive for surface expression of PD-L1 and PD-L1 low or negative cancer comprises fewer than 50% of cancer cells being positive for surface expression of PD-L1.

According to some embodiments, the PD-L1 low or negative cancer is PD-L1 negative cancer comprising less than 1% of cells being positive for surface expression of PD-L1.

According to some embodiments, the trained machine learning algorithm is trained by a method comprising:
at a training stage, training a machine learning algorithm on a training set comprising:
(i) factor expression levels of resistance-associated factors in samples from subjects suffering from cancer and known to be responsive to an anti-PD-1/PD-L1 immunotherapy and factor expression levels of resistance-associated factors in samples from subjects suffering from the cancer and known to be non-responsive to the anti-PD-1/PD-L1 immunotherapy;
(ii) at least one clinical parameter of the subjects known to be responsive and the subjects known to be non-responsive; and
(iii) labels associated with the responsiveness of the subjects suffering from the cancer;
   to produce a trained machine learning algorithm, wherein the trained machine learning algorithm is trained to output the resistance score or response score.

According to some embodiments, the expression levels of resistance-associated factors and the at least one clinical parameter are labeled with the labels.

According to some embodiments, the total resistance score predetermined threshold is 5 and a resistance score above 5 indicates the subject is resistant to the therapy or the total resistance score is converted to a response score by the equation (10-total resistance score) and wherein a response score above a predetermined threshold indicates the subject is responsive to therapy, optionally wherein the response score predetermined threshold is 5.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C: Illustration of protein expression distributions in responders and non-responders populations at the single protein level. (1A-1C) Computer-generated examples of distributions of protein expression for responder and non-responder populations. Example of protein expression levels that may be considered as RAPs (light-gray dashed line) or are not RAPs (dark-gray dashed line) based on the population expression distribution data are shown.

FIG. 2: Illustration of the RAP score of Equation 2 as implemented in Algorithm 1. The RAP score was calculated using synthetic data, where the responder and non-responder populations were generated by sampling from a normal distribution. The synthetic populations expression levels are shown in histograms, the responder population in dark-grey and the non-responder population in light-grey. Given these distributions, the RAP score was calculated for each expression level. The resulting RAP score is plotted in a blue curve, the values are indicated in the secondary Y-axis on the right.

FIGS. 3A-3C: RAP score threshold determination based on AUC as a function of RAP score. AUC at each RAP score was calculated and the peak of the obtained curve was determined as the threshold (dotted line) for determining a certain protein as a RAP or not. (3A-3B) Graph describing determination of the RAP score threshold using the mathematical approach for protein measures at (3A) T1 and (3B) T0. (3C) Graph describing determination of the RAP score threshold using the machine learning approach.

FIGS. 4A-4B: (4A) Bar chart showing the number of RAPs for each patient in the test cohort (n=30). Responders-light-grey; non-responders-dark-grey. (4B) ROC curve for the RAP analysis.

FIG. 5: Heat map of hallmarks of cancer significantly enriched in six patients. The enrichment analysis was based on Fisher exact test (FDR<0.05). Next to each patient identifier the number of the patient's RAPs is indicated in brackets.

FIG. 6: Protein-protein network of the key RAPs in the current cohort. The network is based on STRING database. Each node (protein) is colored based on the hallmarks of cancer to which it is associated. A black circular frame indicates a targetable RAP. The size of each node correlates with the number of patients that had the examined RAP. The compartment/s of each node is indicated in the middle (based on Human Protein Atlas). I, intracellular. M, membranal. S, soluble. A protein can have more than one compartment.

FIG. 7: Chart of the significantly enriched hallmarks of cancer among the 19 RAPs. The analysis was done using Fisher exact test. Enrichment factor above 1 indicates enrichment.

FIG. 8: Heat map of the protein expression levels of the 19 RAPs in healthy tissues. The expression data is based on Human Protein Atlas (HPA) database.

FIG. 9: Heat map of the percentage of medium-high level staining in patients with different cancer types, including NSCLC. The expression data is based on Human Protein Atlas (HPA) database.

FIGS. 10A-10F: Clinical description of the 184 patients included in the analysis. (10A) Heatmap representing patient clinical characteristics: response to treatment (ORR1, ORR2, 1-year DCB); percent of cells expressing PD-L1 in biopsy immunostaining, a prognostic marker of response to treatment; treatment type: ICI only or combined treatment of ICI and chemotherapy; line of treatment: first line indicates ICI treatment was given as the first systemic treatment for NSCLC, advanced line indicates a previous non-ICI treatment was given before the current ICI treatment was administered. Sex indicates patient sex at birth. Histology indicates the lung cancer histological type (ADC-adenocarcinoma, SCC-squamous cell carcinoma). (10B-10C) Violin plots of the correlation of the patient age with response in each time point: (10B) ORR1 and (10C) ORR2. ORR1 and ORR2 are overall response determined 3 months and 6 months following treatment initiation, respectively. (10D-10E) Graphical display of the response groups in (10D) ORR1 and (10E) ORR2. NR=non-responders. R=responders (partial responders or complete responders). SD=stable disease (in the model they are included in the responder group). (10F) Graphical display of the division of the population into the development and the validation sets.

FIGS. 11A-11B: Performance of the classification model. ROC AUC was calculated using the total resistance score together with actual overall response evaluation at 3-month ORR, −6-month ORR and 1 year duration of clinical benefit (DCB) for both TO and T1. Results at TO for the (11A, upper panel) development set and for the (11A, lower panel and 11B, upper panel) validation set are shown. (11B, lower panel) A similar classification model was generated based on T1.

FIGS. 12A-12B: (12A) Patients sorted by their response probability score (calculated as 1-resistance score) based on protein levels at T0. Actual observed response at 3 months ORR is indicated by color for each patient. (12B) Dot plot of the agreement between the predicted response probability based on TO protein expression and observed response probability at either 3 months, 6 months or 1 year. Each point on the graph indicates a specific patient, and the different time points are indicated by different hues and marker types. The black diagonal line indicates the line y=x, the red diagonal line indicates the fitted regression line for all the points and the goodness of fit of the regression (R²) is indicated. The horizontal lines indicate the average observed response probability for the 3 timepoints (color coded) across the entire validation set.

FIGS. 13A-13B: Survival analysis based on prediction results for ORR at 3-months based on TO protein measurements for (13A) PFS and (13B) OS.

FIGS. 14A-14B: (14A) A functional network of all potential RAPs from this analysis. Each node represents a RAP, and the edge between nodes indicates a functional relation. Nodes with a larger size, and protein name provided, indicate investigational new drugs (INDs) in combination with immunotherapy. The nodes are colored based on the protein function. (14B) Functional network for two patients a predicted non-responder (upper) and a predicted responder (lower). RAPs detected for each patient are outlined in black. The non-responder patient had 44 RAPs detected and the responder had 10 RAPS detected.

FIG. 15: Functional differences between RAPs higher in each response group. Each polygon in the Voronoi plot represents a RAP, and the size correlates with the difference between responders and non-responders. While non-responder RAPs are involved in splicing, signaling and cytoskeleton-related processes, the responder RAPs mainly involved proteolysis and cell adhesion. Each color indicates a different overall function.

FIG. 16: A table describing the clinical parameters of the 339 patients included in the analysis.

FIG. 17: Line graphs of patient number at each time point are indicated per response group (NR, non-responder; R, responder) and in total. The patient cohort was divided into development and validation sets.

FIG. 18: Association of clinical parameters with CB at 3, 6 and 12 months. The examined clinical parameters are age, sex, histology type, treatment type, PD-L1 status and ECOG performance status. NSCC, Non-squamous cell carcinoma; SCC, Squamous cell carcinoma; CB, Clinical benefit; NCB, No clinical benefit; ICI, immune checkpoint inhibitor.

FIGS. 19A-19B: Performance of clinical parameter-based predictive models. (19A) Receiver operating characteristics (ROC) plot of the PD-L1-based predictive model. (19B) ROC plot of the predictive clinical model based on PD-L1, sex, ECOG and treatment line. The area under the curve (AUC) values are indicated for each time point. CI, confidence interval.

FIGS. 20A-20B: (20A) Development of the RAP prediction model. A cohort of advanced stage NSCLC patients receiving ICI-based therapy was assembled. Pre-treatment blood samples were obtained, and plasma proteomes were profiled. Clinical benefit (CB) was assessed at 3, 6 and 12 months after starting treatment, and patients were followed up for 2 years. A predictive model for CB was developed for each time point as follows: Proteins displaying differential plasma levels in CB and NCB patient populations were selected for model training using a statistical test. Such proteins are collectively termed Resistance Associated Proteins (RAPs). A predictive model for CB was developed per RAP using a machine learning algorithm. CB predictions inferred from each RAP were summed up to yield a RAP score per patient. RAP scores (total number of active RAPs) were linearly scaled to values between 0 and 1, enabling the conversion of a given patient's RAP score into a CB probability. (20B) Development and validation of the RAP model. The cohort was divided into development and validation sets (75% and 25%, respectively). The development set was randomly divided into train and test sets (75% and 25%, respectively). The train set was used for RAP selection followed by model training resulting in a predictive model per RAP. Clinical benefit (CB) predictions were then generated per RAP for each patient in the test set. CB predictions from all selected RAPs were summed up to yield a RAP score per patient in the test set. The process was repeated 80 times, each time with a random division of development set patients into train and test sets. RAP scores were averaged per patient in the development set and linearly scaled. Model output is CB probability (a value between 0 and 1). The model was then locked and tested on the independent validation set.

FIG. 21: The effect of RAP number on model performance per time point. Different numbers of RAPs (ranging from 1-400) were selected. For each number, the model was run 10 times. Model performance was assessed by ROC analysis. AUC is indicated. Based on this analysis, 50 was set as the cut-off for the number of selected RAPs.

FIGS. 22A-22G: RAP identification during model development. (22A) Histograms showing the number of identified RAPs grouped according to the number of times they were selected over 80 iterations. The top, middle and bottom histograms are for the 3-, 6- and 12-month time points, respectively. (22B) The total number of RAPs identified per time point. Some proteins measured by the SomaScan assay are redundant due to different aptamers binding to the same protein. The numbers of overall and non-redundant RAPs are indicated by blank and dotted bars, respectively. The numbers of non-redundant RAPs identified at least 40 times in a total of 80 iterations are indicated by lined bars. (22C) A Venn diagram showing the number of RAPs identified per time point. (22D) Hierarchical clustering-based heatmap showing the number of iterations in which a given protein was classified as a RAP. (22E) RAP cellular localization and potential cellular origin. Data were obtained from Human Protein Atlas. The same protein may be assigned more than one cellular localization. (22F) Voronoi plots displaying the main biological functions of the RAPs per time point. Each polygon represents a RAP, and the size correlates with the number of times that the protein was selected as a RAP. Proteins from the same KEGG biological process are grouped together (using default settings of the Proteomaps tool). (22G) Enrichment analysis of RAPs per time point. The enrichment analysis was performed for RAPs that were selected in at least 10 iterations. Fisher exact test (FDR<0.1) was used.

FIGS. 23A-23E: Performance of the RAP predictive model. (23A) Bar plot showing predicted clinical benefit (CB) probabilities sorted from lowest to highest. Observed CB and no CB (NCB) patients are shown as light and dark blue bars, respectively. (23B) Overall survival analysis of patients stratified to high and low CB probability groups. The median CB probability per time point was used as the stratification threshold. HR, hazard ratio. CI, confidence interval. (23C) Progression-free survival analysis of patients stratified to high and low CB probability groups. The median CB probability per time point was used as the stratification threshold. HR, hazard ratio. CI, confidence interval. (23D) Predicted CB probability as a function of observed CB rate. Each dot represents a patient. The observed CB rate for each predicted CB probability datapoint refers to the proportion of observed CB patients within a patient group assigned the CB probability+0.15. X=Y is indicated by a black line. The goodness of fit is indicated. (23E) Receiver operating characteristics (ROC) plot for the RAP model per time point. The area under the curve (AUC) is indicated. The dashed line indicates AUC=0.5. CI, confidence interval.

FIG. 24: Enrichment analysis for CB probabilities and observed CB rates at each time point. The enrichment analysis was done using 2D-enrichment test. The X-axis indicates the enrichment score for predicted CB probability. The Y-axis indicates the enrichment score for observed rates (as defined by the proportion of observed CB patients within a patient group assigned the CB probability+0.15). The enrichment score is a value between 1 and −1. Positive and negative enrichment scores indicate enrichment in high and low CB probabilities, respectively and in high and low observed CB rates, respectively. The solid line indicates the X=Y line.

FIGS. 25A-25D: (25A-25C) Comparison between CB probabilities at sequential time points. Each dot represents a patient in the cohort. CB probability at one time point is plotted against CB probability at a subsequent time point. The colors indicate patient CB labels per time point, and whether the clinical benefit label changed between time points. (25A) Comparison between 3 and 6 months. (25B) Comparison between 3 and 12 months. (25C) Comparison between 6 and 12 months. (25D) Sankey plot displaying the flow of CB labeling over time. CB, Clinical benefit; NCB, No clinical benefit; NA, not available.

FIGS. 26A-26B: The RAP model outperforms PD-L1 and clinical parameter-based models. Predictive performance was compared across five models: RAP model; PD-L1-based model (PD-L1); Clinical model (CM); Integrated RAP+PD-L1; Integrated RAP+CM. (26A) Receiver operating characteristics (ROC) plots of the five models at each time point. The area under the curve (AUC) is indicated. The dashed line indicates AUC=0.5. CI, confidence interval. (26B) Forest plot comparing the five models. Top, Cox regression analysis based on overall survival (OS) data. Bottom, Cox regression analysis based on progression-free survival (PFS) data.

FIG. 27: RAP model performance in different patient subsets. NSCC, non-squamous cell carcinoma; SCC, squamous cell carcinoma.

FIG. 28: Kaplan-Meier plots of PD-L1-high, PD-L1-low and PD-L1-negative patients in the overall cohort. Left, overall survival (OS); right, progression-free survival (PFS). Dashed line indicates median survival.

FIG. 29: The RAP model predicts differential survival outcomes in patients with PD-L1≥50%. PD-L1-high patients were stratified to high (left) and low (right) CB probability groups using the cohort median CB probability as the stratification threshold. Overall survival (OS; lower panel) and progression-free survival (PFS; upper panel) were evaluated in patients treated with ICI monotherapy vs combination ICI-chemotherapy. Dashed line indicates median survival.

FIG. 30: The RAP model predicts differential survival outcomes in patients with PD-L1≤50%. PD-L1-low and PD-L1-negative patients (PD-L1-low-negative) were stratified to high (left) and low (right) CB probability groups using the cohort median CB probability as the stratification threshold. Overall survival (OS; lower panel) and progression-free survival (PFS; upper panel) were evaluated in patients treated with ICI monotherapy vs combination ICI-chemotherapy. Dashed line indicates median survival.

FIG. 31A-31C: (31A) Patient clinical data. (31B) Development of the PROphet prediction model. A cohort of advanced stage NSCLC patients receiving ICI-based therapy was assembled. Pre-treatment blood samples were obtained, and plasma proteomes were profiled using SomaScan technology. Clinical benefit (CB) was assessed at 12 months after starting treatment, and patients were followed up for 2 years. A predictive model for CB was developed as follows: Proteins displaying differential plasma levels in CB and NCB patient populations were selected for model training using a statistical test. Such proteins are collectively termed Resistance Associated Proteins (RAPs). A predictive model for CB was developed per RAP using a machine learning algorithm. CB predictions inferred from each RAP were summed up to yield a RAP score per patient. RAP scores were linearly scaled to values between 0 and 1, enabling the conversion of a given patient's RAP score into a CB probability, which determines the PROphet result, negative or positive on the scale of 0 and 10. (31C) Development and validation of the RAP model. The cohort was divided into development and validation sets. The development set was randomly divided into train and test sets (75% and 25%, respectively). The train set was used for RAP selection followed by model training resulting in a predictive model per RAP. Clinical benefit (CB) predictions were then generated per RAP for each patient in the test set. CB predictions from all selected RAPs were summed up to yield a RAP score per patient in the test set. The process was repeated 80 times, each time with a random division of development set patients into train and test sets. RAP scores were averaged per patient in the development set and linearly scaled. Model output is CB probability (a value between 0 and 1) that is translated to PROphet score. The model was then locked and tested on the independent validation set.

FIGS. 32A-32D: The PROphet predicts overall survival for patients receiving ICI-based therapy and outperforms PD-L1 based prediction. (32A) Kaplan-Meier plot for PD-L1≥50% versus PD-L1<50%. (32B) Predicted CB probability based on PD-L1 prediction as a function of observed CB rate. Each dot represents a patient. The observed CB rate for each predicted CB probability datapoint refers to the proportion of observed CB patients within a patient group assigned CB probability±0.04. The goodness of fit (R2) is indicated. (32C) Kaplan-Meier plot for patient stratification based on PROphet model. (32D) Predicted CB probability based on PROphet model as a function of observed CB rate. The observed CB rate for each predicted CB probability datapoint refers to the proportion of observed CB patients within a patient group assigned CB probability+0.05.

FIGS. 33A-33B: PROphet is not predictive for chemotherapy patients. (33A) Kaplan-Meier plot for chemotherapy patients classified as PROphet positive or negative, with no significant difference between these two subgroups. (33B) Predicted CB probability based on PROphet model as a function of observed CB rate. Each dot represents a patient. The observed CB rate for each predicted CB probability datapoint refers to the proportion of observed CB patients within a patient group assigned CB probability+0.05. The goodness of fit (R²) is indicated.

FIG. 34: Flowchart of the patients participating in the PROphet+PD-L1 analysis.

FIGS. 35A-35H: The PROphet model predicts differential overall survival outcome between different subgroups when combined with PD-L1 expression level. (35A-35C) Kaplan-Meier plots for PROphet-positive prediction with PD-L1≥50% patients (35A), PD-L1 1-49% (35B) and PD-L1<1% (35C). In 35A, PD-L1≥50% patients received either ICI-chemotherapy combination therapy or ICI monotherapy. In 35B, and 35C, PD-L1 1-49% and PD-L1<1% patients that received ICI-chemotherapy combination were compared to patients receiving chemotherapy alone. (35D-35F) Kaplan-Meier plots for PROphet®-negative prediction with PD-L1≥50% patients (35D), PD-L1 1-49% (35E) and PD-L1<1% (35F). In 35D, PD-L1≥50% patients received either ICI-chemotherapy combination therapy or ICI monotherapy. In 35E, and 35F, PD-L1 1-49% and PD-L1<1% patients that received ICI-chemotherapy combination were compared to patients receiving chemotherapy alone. HR, hazard ratio. CI, confidence interval. (35G-35H) Kaplan Meier plots for PROphet positive (35G) or PROphet negative (35H) patients with PD-L1 expression level of 1-49%. The ICI-chemotherapy combination is compared either to ICI-monotherapy or to chemotherapy alone. HR, hazard ratio. CI, confidence interval.

FIGS. 36A-36F: The PROphet model predicts differential progression free survival outcome between different subgroups when combined with PD-L1 expression level. (36A-36C) Kaplan-Meier plots for PROphet-positive prediction with PD-L1≥50% patients (36A), PD-L1 1-49% (36B) and PD-L1<1% (36C). In 36A, PD-L1≥50% patients received either ICI-chemotherapy combination therapy or ICI monotherapy. In 36B, and 36C, PD-L1 1-49% and PD-L1<1% patients that received ICI-chemotherapy combination were compared to patients receiving chemotherapy alone. (36D-36F) Kaplan-Meier plots for PROphet®-negative prediction with PD-L1>50% patients (36D), PD-L1 1-49% (36E) and PD-L1<1% (36F). In 36D, PD-L1≥50% patients received either ICI-chemotherapy combination therapy or ICI monotherapy. In 36E, and 36F, PD-L1 1-49% and PD-L1<1% patients that received ICI-chemotherapy combination were compared to patients receiving chemotherapy alone. HR, hazard ratio. CI, confidence interval.

FIGS. 37A-37C: Forest plot for multivariate analysis of the PROphet model. (37A) PD-L1>50% patients. (37B) PD-L1 1-49% patients. (37C) PD-L1<1% patients.

FIGS. 38A-38D: Comparison between PROphet-positive and -negative results. (38A-38B) Comparison for PD-L1≥50% patients. (38A), Patients receiving ICI monotherapy. (38B) Patients receiving ICI-chemotherapy combination. (38C) Comparison for PD-L1 1-49% patients receiving ICI-chemotherapy combination. (38D) Comparison for PD-L1<1% patients receiving ICI-chemotherapy combination.

FIGS. 39A-39H Applicability of the response prediction using the PROphet model in patients with melanoma, SCLC, and HPV-related malignancies. (39A-C) PROphet model prediction in melanoma cohort. (39A) Model ROC AUC for 1-year durable clinical benefit. (39B) Predicted response probability based on the PROphet model versus observed response probability. Each point indicates a specific patient. (39C) Kaplan Meier plots for PROphet positive and PROphet negative patients. (39D) Kaplan-Meier curves showing survival of PROphet positive and PROphet negative patients with SCLC. (39E-G) Kaplan-Meier curves showing survival of PROphet positive and PROphet negative patients with HPV-related malignancies including (39E) anogenital SCC, (39F) cervical cancer, and (39G) head and neck cancer. (39H) Kaplan-Meier curve for all HPV-related malignancies. Hazard ratio with 95% confidence intervals and p-values are indicated.

FIGS. 40A-40B: Applicability of the response prediction using the PROphet model for NSCLC patients with targetable mutations. (40A) Correlation between the PROphet score and overall survival duration of NSCLC patients with targeted mutations treated with PD-1 inhibitors. R²-0.41, p=0.0073. (40B) Kaplan-Meier curves showing survival of PROphet positive and PROphet negative patients, HR=0.36, p=0.07.

### DETAILED DESCRIPTION OF THE INVENTION

The present invention, in some embodiments, provides methods of predicting response of a subject comprising a tumor with high, low or negative levels of PD-L1 to immunotherapy. Here, we developed a novel and inherently robust machine learning (ML)-based model that analyzes proteomic profiles in pre-treatment blood plasma to predict benefit from ICI therapy in cancer patients. By integrating predictions from a large collection of proteomic biomarkers, the model accurately predicts clinical benefit at three time points along the treatment course and stratifies patients according to survival outcomes, or PFS, outperforming PD-L1-based prediction. Furthermore, the model shows potential for further optimizing treatment selection when used together with PD-L1 classification. Overall, the model provides clinically valuable information to support treatment decisions in cancer.

The invention is based, at least in part on the discovery of a novel tool for supporting treatment decision for cancer patients receiving ICI-based therapy. The RAP (PROphet) model provides two main clinical utilities. First, it successfully predicts therapeutic benefit at 12 months, displaying superior predictive capabilities over PD-L1 based models. Second, when used in combination with PD-L1 testing, the model helps in determining whether a patient should receive ICI alone or an ICI-chemotherapy combination. Specifically, subjects with high PD-L1 levels and a high total response score are predicted to respond to ICI as a monotherapy and need not be exposed to the adverse side effects resultant from chemotherapy. Subjects with high PD-L1 but a low total response score are advised to proceed with combination ICI-chemotherapy. In patients with low PD-L1 but a high total response score, treatment with combination ICI-chemotherapy is predicted to be effective, but patients with low PD-L1 and low total response score would be advised to consider alternative therapies.

By a first aspect, there is provided a method of predicting response of a subject suffering from a PD-L1 high cancer to a monotherapy comprising immunotherapy, the method comprising
a. receiving expression levels for a plurality of factors
   i. in a population of subjects known to respond to the therapy (responders);
   ii. in a population of subjects known to not respond to the therapy (non-responders); and
   iii. in the subject;
b. calculate for factors of the plurality of factors a resistance score, wherein the calculating comprises applying a machine learning algorithm and wherein the machine learning algorithm outputs the resistance score; and
c. combine the calculated resistance scores to produce a total resistance score;
   wherein a subject with a total resistance score beyond a predetermined threshold is predicted to not respond to the monotherapy and a subject with a total resistance score within the predetermined threshold is predicted to respond to the monotherapy;
   thereby predicting the response of a subject to a monotherapy.

By another aspect, there is provided a method of predicting response of a subject suffering from a PD-L1 low or negative cancer to a combination therapy comprising immunotherapy and chemotherapy, the method comprising
a. receiving expression levels for a plurality of factors
   i. in a population of subjects known to respond to the therapy (responders);
   ii. in a population of subjects known to not respond to the therapy (non-responders); and
   iii. in the subject;
b. calculate for factors of the plurality of factors a resistance score, wherein the calculating comprises applying a machine learning algorithm and wherein the machine learning algorithm outputs the resistance score; and
c. combine the calculated resistance scores to produce a total resistance score;
   wherein a subject with a total resistance score beyond a predetermined threshold is predicted to not respond to the combination therapy and a subject with a total resistance score within the predetermined threshold is predicted to respond to the combination therapy;
   thereby predicting the response of a subject to the combination therapy.

By another aspect, there is provided a method of predicting response of a subject to a therapy, the method comprising:
a. receiving expression levels for a plurality of factors
   i. in a population of subjects known to respond to the therapy (responders);
   ii. in a population of subjects known to not respond to the therapy (non-responders); and
   iii. in the subject;
b. calculate for at least one factor of the plurality of factors a resistance score; and
c. classify a factor with a resistance score beyond a threshold as a resistance-associated factor;
   wherein a subject with a number of resistance-associated factors beyond a predetermined number is predicted to be resistant to the therapy, thereby predicting the response of a subject to a therapy.

By another aspect, there is provided a method of predicting response of a subject to a therapy, the method comprising:
a. receiving expression levels for a plurality of factors
   i. in a population of subjects known to respond to the therapy (responders);
   ii. in a population of subjects known to not respond to the therapy (non-responders); and
   iii. in the subject;
b. calculate for at least one factor of the plurality of factors a resistance score;
c. classify a factor with a resistance score beyond a threshold as a resistance-associated factor;
d. sum the number resistance-associated factors; and
e. apply a trained machine learning algorithm to the number of resistance-associated factors and at least one clinical parameter, wherein the trained machine learning algorithm outputs a total resistance score and a total resistance score beyond a predetermined threshold indicates the subject is resistant to the therapy;
thereby predicting the response of a subject to a therapy.

By another aspect, there is provided a method of predicting response of a subject to a therapy, the method comprising:
a. receiving expression levels for a plurality of factors
   i. in a population of subjects known to respond to the therapy (responders);
   ii. in a population of subjects known to not respond to the therapy (non-responders); and
   iii. in the subject;
b. calculate for factors of the plurality of factors a resistance score, wherein the resistance score is based on the similarity of the factor expression level in the subject to the factor expression level in the responders and the similarity to the factor expression level in the subject to the factor expression level in the non-responders and wherein the calculating comprises applying a trained machine learning algorithm that outputs the resistance score; and
c. sum the calculated resistance scores to produce a total resistance score;
   wherein a subject with a total resistance score beyond a predetermined threshold is predicted to be resistant to said therapy;
   thereby predicting the response of a subject to a therapy.

By another aspect, there is provided a method comprising:
at a training stage, training a machine learning algorithm on a training set comprising:
   (i) a number of resistance-associated factors expressed in samples from subjects suffering from a disease and known to be responsive to a therapy and from subjects suffering from the disease and known to be non-responsive to the therapy;
   (ii) at least one clinical parameter of the subjects; and
   (iii) labels associated with the responsiveness of the subjects;
to produce a trained machine learning algorithm

By another aspect, there is provided a method comprising:
at a training stage, training a machine learning algorithm on a training set comprising:
   (i) factor expression levels of resistance-associated factors in samples from subjects suffering from a disease and known to be responsive to a therapy and from subjects suffering from the disease and known to be non-responsive to the therapy;
   (ii) at least one clinical parameter of the subjects; and
   (iii) labels associated with the responsiveness of the subjects;
   to produce a trained machine learning algorithm.

In some embodiments, the method is a diagnostic method. In some embodiments, the method is an in vitro method. In some embodiments, the method is an ex vivo method. In some embodiments, the method is a computer implemented method. In some embodiments, the method is a statistical method. In some embodiments, the method is a method that cannot be performed in a human mind. In some embodiments, the method is a computerized method. In some embodiments, the processor is a computer processor. In some embodiments, the processor is a computer.

In some embodiments, the method is for predicting response to therapy. In some embodiments, the method is for determining response to therapy. In some embodiments, the method is for determining response score. In some embodiments, the method is for determining response probability. In some embodiments, a response probability is a response score. In some embodiments, the method is for determining clinical benefit probability. In some embodiments, the method is for determining overall survival. In some embodiments, the method is for determining progression free survival (PFS). In some embodiments, the method is for determining overall survival (OS). In some embodiments, the method is for determining survival probability. In some embodiments, determining is predicting. According to some embodiments, resistance score is determined. According to other embodiments, prediction of resistance probability is determined. According to some other embodiments, resistance probability below 20% indicates the subject is responsive to therapy. According to some other embodiments, resistance probability below 50% indicates the subject is responsive to therapy. According to some embodiments, response score is determined. According to other embodiments, prediction of response probability is determined. According to some other embodiments, response probability beyond 80% indicates the subject is responsive to therapy. According to some other embodiments, response probability beyond 50% indicates the subject is responsive to therapy. In some embodiments, beyond is above. In some embodiments, beyond is below. It will be understood by a skilled artisan that a scale can be designed to be measured in either direction and so above/below depends on the construction of the scale.

In some embodiments, the method is for determining if a subject is a responder to the therapy. In some embodiments, the method is for determining if a subject is a non-responder to the therapy. In some embodiments, the method is for predicting a subject's response to therapy. In some embodiments, the method is for monitoring response to the therapy. In some embodiments, the method is for determining if the therapy should continue, be adjusted (e.g., by further treating the subject with an additional therapy including but not limited to an agent determined by the RAP analysis provided hereinbelow) or changed. In some embodiments, the method is for determining a subject as being a responder to the therapy, or a non-responder to the therapy. In some embodiments, the method is for determining a subject as being a responder to the therapy, a non-responder to the therapy, or as having a stable diseased state. In some embodiments, the method is for predicting if a subject will respond to the therapy, or not respond to the therapy. In some embodiments, a responder is a responder to a monotherapy (mono-responder). In some embodiments, a responder is a responder to combination therapy (combo-responder). In some embodiments, a non-responder is a non-responder to monotherapy (mono-non-responder). In some embodiments, a non-responder is a non-responder to combination therapy (combo-non-responder). In some embodiments, the method is for determining if the subject will benefit or not benefit from the treatment.

In some embodiments, non-response comprises progressive disease. In some embodiments, non-response comprises cancer progression. In some embodiments, non-response comprises stable disease. In some embodiments, non-response comprises a worsening of symptoms of the disease. In some embodiments, non-response is not the development of side effects. In some embodiments, non-response comprises growth, metastasis and/or continued proliferation of a cancer. In some embodiments, non-response comprises no clinical benefit (NCB). In some embodiments, non-response is non-survival. In some embodiments, non-response is non-survival and/or cancer progression. In some embodiments, response is stable disease. In some embodiments, response comprises remission. In some embodiments, remission is minimal remission. In some embodiments, remission is partial remission. In some embodiments, remission is complete remission. In some embodiments, response is survival. In some embodiments, response is progression free survival. In some embodiments, response is long progression free survival. In some embodiments, response is measured using the overall response rate (ORR). A trained physician will be familiar with methods of determining response and specifically the ORR. In some embodiments, response is measured using Response Evaluation Criteria In Solid Tumors (RECIST). In some embodiments, response comprises survival. In some embodiments, survival is overall survival. In some embodiments, survival is progression free survival. In some embodiments, survival is overall survival. In some embodiments, response comprises a clinical benefit (CB). In some embodiments, response comprises a durable clinical benefit (DCB). In some embodiments, CB is DCB. In some embodiments, CB is PFS. In some embodiments, CB is PFS at 12 months after the commencement of treatment. In some embodiments, CB is PFS at 7 months after the commencement of treatment. In some embodiments, the population of subject known to respond and known not to respond are determined based on PFS and the predicted response comprises OS. In some embodiments, PFS is PFS at 12 months. In some embodiments, PFS is PFS at 7 months. In some embodiments, PFS is PFS at 6 months. In some embodiments, PFS is PFS at 3 months. In some embodiments, OS is OS at 12 months. In some embodiments, OS is OS at 7 months. In some embodiments, OS is OS at 6 months. In some embodiments, OS is OS at 3 months. In some embodiments, no clinical benefit or non-clinical benefit is the absent of a clinical benefit described herein.

In some embodiments, the subject is a mammal. In some embodiments, the subject is a human. In some embodiments, the subject suffers from a disease. In some embodiments, the disease is treatable by the therapy. In some embodiments, the disease is cancer. In some embodiments, the disease is treatable by an immune checkpoint inhibitor (ICI). In some embodiments, the cancer is a PD-L1 positive cancer. In some embodiments, the cancer is a PD-L1 high cancer. In some embodiments, the cancer is a PD-L1 low cancer. In some embodiments, the cancer is a PD-L1 negative cancer. In some embodiments, the cancer is a PD-L1 low or negative cancer. In some embodiments, the cancer is solid cancer. In some embodiments, the cancer is a tumor. In some embodiments, the cancer is selected from hepato-biliary cancer, cervical cancer, urogenital cancer (e.g., urothelial cancer), anogenital, testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer, leukemia and lymphoma. In some embodiments, the cancer is selected from skin cancer, and lung cancer. In some embodiments, the cancer is skin cancer. In some embodiments, the cancer is lung cancer. In some embodiments, the skin cancer is melanoma. In some embodiments, the lung cancer is small cell lung cancer. In some embodiments, the lung cancer is non-small cell lung cancer. In some embodiments, the melanoma is non-resectable melanoma. In some embodiments, the melanoma is metastatic melanoma. In some embodiments, the cancer is an HPV (Human Papilloma Virus) positive cancer. In some embodiments, the cancer is an HPV-related cancer. In some embodiments, the cancer is anogenital cancer. In some embodiments, the anogenital cancer is anogenital squamous-cell carcinoma (SCC). In some embodiments, anogenital cancer comprises anal, cervical, penile, vaginal, and vulvar cancer. In some embodiments, the cancer is cervical cancer. In some embodiments, the cervical cancer is small-cell cervical cancer. In some embodiments, the cancer is a head and neck cancer. In some embodiments, the head and neck cancer is head and neck SCC (HNSCC). In some embodiments, the cancer is selected from lung cancer, skin cancer, anogenital cancer, cervical cancer and head and neck cancer.

In some embodiments, the cancer is resistant to a therapy. In some embodiments, the therapy is a non-immunotherapy. In some embodiments, the therapy is another therapy. In some embodiments, the therapy is targeted therapy. In some embodiments, the therapy is not anti-PD-1/L1 immunotherapy. In some embodiments, the cancer is resistant to a targeted therapy. In some embodiments, the targeted therapy is a tyrosine kinase inhibitor (TKI). In some embodiments, the subject has been previously treated with a TKI. In some embodiments, the subject was treated with and found resistant to a TKI. In some embodiments, the method is a method of determining if a subject resistant to a targeted therapy will respond to a PD-1/L1 immunotherapy. In some embodiments, the subject comprises a TKI resistant cancer. In some embodiments, cancer is a TKI resistant NSCLC. In some embodiments, the cancer comprises a mutation of a tyrosine kinase receptor gene. In some embodiments, the tyrosine kinase receptor gene is selected from epidermal growth factor receptor (EGFR), Anaplastic lymphoma kinase (ALK) and Proto-oncogene tyrosine-protein kinase ROS (ROS1).

In some embodiments, the subject is naïve to therapy before the first determining. In some embodiments, the subject has not received the therapy before the first determining. In some embodiments, the subject has received the therapy previously. In some embodiments, the subject has previously been treated by a therapy other than the therapy. In some embodiments, the subject is simultaneously treated by a therapy other than the therapy. In some embodiments, the other therapy is a TGFB-trap fusion protein. In some embodiments, the other therapy is tyrosine kinase inhibitor. In some embodiments, the subject is naïve to any therapy. In some embodiments, the subject is naïve to immunotherapy. In some embodiments, the therapy is the first line of treatment. In some embodiments, the therapy is an advanced line of treatment.

In some embodiments, the therapy is an anticancer therapy. In some embodiments, the anticancer therapy is radiation. In some embodiments, the anticancer therapy is chemotherapy. In some embodiments, the therapy is immunotherapy. In some embodiments, the anticancer therapy is immunotherapy. In some embodiments, the anticancer therapy is targeted therapy. In some embodiments, the anticancer therapy is selected from radiation, chemotherapy, immunotherapy, targeted therapy, hormonal therapy, anti-angiogenic therapy and photodynamic therapy, thermotherapy, surgery, and a combination thereof. In some embodiments, the immunotherapy is selected from immune checkpoint inhibition, immune checkpoint modulation, immune checkpoint blockade, adoptive-cell transfer therapy, oncolytic virus therapy, vaccine therapy, immune system modulation and therapy using monoclonal antibodies. In some embodiments, an immunotherapy is selected from immune checkpoint inhibitors, immune checkpoint modulators, immune checkpoint blockers, adoptive-cell transfer therapy, oncolytic virus therapy, treatment vaccines, immune system modulators and monoclonal antibodies. In some embodiments, the immunotherapy is an immune checkpoint inhibitor. In some embodiments, the immunotherapy is immune checkpoint blockade. In some embodiments, the targeted therapy is tyrosine kinase inhibitors. In some embodiments, the targeted therapy is a TGFB-trap fusion protein.

In some embodiments, an immunotherapy is administered in combination with one or more conventional cancer therapy including chemotherapy, targeted therapy, steroids, and radiotherapy. Combinations of ICI and chemotherapy/radiotherapy/targeted therapy have been studied in multiple clinical trials. It will be understood by a skilled artisan that the predictive proteins disclosed herein are predictive in immunotherapy as a monotherapy, as well as part of a combination therapy. In some embodiments, the therapy is a monotherapy.

In some embodiments, the monotherapy comprises an immunotherapy. In some embodiments, the monotherapy consists of immunotherapy. In some embodiments, the monotherapy does not comprise chemotherapy. In some embodiments, the monotherapy is an anti-PD-1/PD-L1 immunotherapy. In some embodiments, the therapy is a combination therapy. In some embodiments, the combination therapy comprises an immunotherapy and another therapy. In some embodiments, the combination therapy comprises an immunotherapy and a chemotherapy. In some embodiments, the combination therapy comprises an immunotherapy and a targeted therapy. In some embodiments, the targeted therapy is a tyrosine kinase inhibitor. In some embodiments, the targeted therapy is an anti-transforming growth factor beta (TGFB) agent. In some embodiments, the TGFB agent is a TGFB-trap fusion protein. TGFB-trap fusion proteins are well-known in the art and are disclosed for example in Knudson et al., “M7824, a novel bifunctional anti-PD-L1/TGFB Trap fusion protein, promotes anti-tumor efficacy as monotherapy and in combination with vaccine”, Oncoimmunology. 2018 Feb. 14; 7 (5): e1426519 and Morris et al., “Bintrafusp alfa, an anti-PD-L1: TGF-β trap fusion protein, in patients with ctDNA-positive, liver-limited metastatic colorectal cancer”, Cancer Res Commun. 2022 September; 2 (9): 979-986, the contents of which are hereby incorporated by reference in their entirety. In some embodiments, the combination therapy further comprises radiation. In some embodiments, the combination therapy further comprises a non-anti-PD-1/PD-L1 immunotherapy. In some embodiments, the anti-PD-1/PD-L1 immunotherapy is selected from Pembrolizumab, Nivolumab, Durvalumab and Atezolizumab. In some embodiments, the anti-PD-1/PD-L1 immunotherapy is selected from Pembrolizumab, Nivolumab, Durvalumab, Atezolizumab, and Cemiplimab. In some embodiments, the immunotherapy comprises Pembrolizumab. In some embodiments, the immunotherapy comprises Nivolumab. In some embodiments, the immunotherapy comprises Durvalumab. In some embodiments, the immunotherapy comprises Atezolizumab. In some embodiments, the chemotherapy is selected from Carboplatin, Paclitaxel, Nab-Paclitaxel, Pemetrexed, Vinorelbine, and Cisplatin. In some embodiments, the chemotherapy is selected from Carboplatin, Paclitaxel, Nab-Paclitaxel, Pemetrexed, Vinorelbine, Cisplatin, dacarbazine, temozolomide, albumin-bound paclitaxel, and vinblastine. In some embodiments, the chemotherapy is Carboplatin. In some embodiments, the chemotherapy is Paclitaxel. In some embodiments, the chemotherapy is Nab-Paclitaxel. In some embodiments, the chemotherapy is Pemetrexed. In some embodiments, the chemotherapy is Vinorelbine. In some embodiments, the chemotherapy is Cisplatin. In some embodiments, the combination therapy comprises Carboplatin, Durvalumab, and Paclitaxel. In some embodiments, the combination therapy comprises Atezolizumab, Bevacizumab, Carboplatin, and Paclitaxel. In some embodiments, the combination therapy comprises Carboplatin, Nab-Paclitaxel, and Pembrolizumab. In some embodiments, the combination therapy comprises Carboplatin, Nivolumab, and Paclitaxel. In some embodiments, the combination therapy comprises Carboplatin, Paclitaxel, Pembrolizumab. In some embodiments, the combination therapy comprises Carboplatin, Nivolumab, Pemetrexed. In some embodiments, the combination therapy comprises Carboplatin, Paclitaxel, Pembrolizumab, and radiation. In some embodiments, the combination therapy comprises Carboplatin, and Pembrolizumab. In some embodiments, the combination therapy comprises Carboplatin, Pembrolizumab, and Pemetrexed. In some embodiments, the combination therapy comprises Carboplatin, Pembrolizumab, and Vinorelbine. In some embodiments, the combination therapy comprises Cisplatin, Pembrolizumab, and Pemetrexed. In some embodiments, the combination therapy comprises an anti-CTLA-4 antibody. In some embodiments, the CTLA-4 antibody is ipilimumab. In some embodiments, the CTLA-4 antibody is Tremelimumab. In some embodiments, the combination therapy comprises an anti-LAG3 antibody. In some embodiments, the LAG3 antibody is relatlimab. In some embodiments, the TKI is selected from Osimertinib, Erlotinib, Afatinib, Gefitinib, Dacomitinib, dacomitinib, Amivantamab-vmjw, Mobocertinib, Sotorasib, Adagrasib, Alectinib, Brigatinib, Lorlatinib, Ceritinib, Crizotinib, entrectinib, Dabrafenib, ceritinib, trametinib, Vemurafenib, Tepotinib, Capmatinib, Selpercatinib, Pralsetinib, Fam-trastuzumab, deruxtecan-nxki, Ado-trastuzumab, emtansine, Cabozantinib, Ado-trastuzumab emtansine, Larotrectinib, alectinib, Cetuximab, cobimetinib, Encorafenib, binimetinib, Lenvatinib, imatinib, dasatinib, nilotinib, and ripretinib.

The NCCN guidelines for 2023 provide the following lists of treatment which may be used alone or in combination to treat NSCLC, Melanoma or SCLC the are as follows: NSCLC-ICI: Atezolizumab, pembrolizumab, Durvalumab, nivolumab, ipilimumab, Cemiplimab, Cemiplimab-rwlc, and Tremelimumab. TKIs: Osimertinib, Erlotinib, Afatinib, Gefitinib, Dacomitinib, dacomitinib, Amivantamab-vmjw, Mobocertinib, Sotorasib, Adagrasib, Alectinib, Brigatinib, Lorlatinib, Ceritinib, Crizotinib, entrectinib, Dabrafenib, ceritinib, trametinib, Vemurafenib, Tepotinib, Capmatinib, Selpercatinib, Pralsetinib, Fam-trastuzumab, deruxtecan-nxki, Ado-trastuzumab, emtansine, Cabozantinib, Ado-trastuzumab emtansine, Larotrectinib, alectinib, and Cetuximab. Anti-VEGF: Ramucirumab, and bevacizumab. Chemotherapy: Carboplatin, paclitaxel, pemetrexed, gemcitabine, Cisplatin, docetaxel, vinorelbine, etoposide, and albumin-bound paclitaxel. Melanoma-ICI: Nivolumab, Pembrolizumab, Ipilimumab, and relatlimab. Targeted therapy: Dabrafenib, trametinib, Vemurafenib, cobimetinib, Encorafenib, binimetinib, and lenvatinib. KIT inhibitors: imatinib, dasatinib, nilotinib, and ripretinib. ROS1 fusions drugs: Crizotinib, and entrectinib. NTRK fusions drugs: Larotrectinib, and entrectinib. NRAS drugs: Binimetinib. Chemotherapy: dacarbazine, temozolomide, albumin-bound paclitaxel, carboplatin, paclitaxel, cisplatin, vinblastine, and dacarbazine. SCLC-Chemotherapy: Cisplatin, etoposide, Carboplatin, irinotecan, Topotecan, Lurbinectedin, Cyclophosphamide, doxorubicin, vincristine, Docetaxel, Gemcitabine, Temozolomide, Vinorelbine, Bendamustine, platinum, and paclitaxel. ICI: atezolizumab, durvalumab, nivolumab, pembrolizumab, and ipilimumab.

In some embodiments, the immunotherapy is a plurality of immunotherapies. In some embodiments, the immunotherapy is immune checkpoint blockade. In some embodiments, the immunotherapy is immune checkpoint protein inhibition. In some embodiments, the immunotherapy is immune checkpoint protein modulation. In some embodiments, the immunotherapy comprises immune checkpoint inhibition. In some embodiments, the immunotherapy comprises immune checkpoint modulation. In some embodiments, immune checkpoint blockade and/or immune checkpoint inhibition comprises administering to the subject an immune checkpoint inhibitor. In some embodiments, inhibition comprises administering an immune checkpoint inhibitor. In some embodiments, the inhibitor is a blocking antibody. In some embodiments, the immunotherapy comprises immune checkpoint blockade. In some embodiments, modulation comprises administering an immune checkpoint modulator. In some embodiments, immune checkpoint modulation comprises administering to the subject an immune checkpoint modulator.

As used herein, the term “an immune checkpoint inhibitor (ICI)” refers to a single ICI, a combination of ICIs and a combination of an ICI with another cancer therapy. The ICI may be a monoclonal antibody, a dual-specific antibody, a humanized antibody, a fully human antibody, a fusion protein, or a combination thereof directed to blocking, inhibition or modulation of immune checkpoint proteins. In some embodiments, an immune checkpoint inhibitor is an immune checkpoint modulator. In some embodiments, an immune checkpoint inhibitor is an immune checkpoint blocker. In some embodiments, the immune checkpoint protein is selected from PD-1 (Programmed Death-1); PD-L1; PD-L2; CTLA-4 (Cytotoxic T-Lymphocyte-Associated protein 4); A2AR (Adenosine A2A receptor), also known as ADORA2A; B7-H3, also called CD276; B7-H4, also called VTCN1; B7-H5; BTLA (B and T Lymphocyte Attenuator), also called CD272; IDO (Indoleamine 2,3-dioxygenase); KIR (Killer-cell Immunoglobulin-like Receptor); LAG-3 (Lymphocyte Activation Gene-3); TDO (Tryptophan 2,3-dioxygenase); TIM-3 (T-cell Immunoglobulin domain and Mucin domain 3); VISTA (V-domain Ig suppressor of T cell activation); NOX2 (nicotinamide adenine dinucleotide phosphate NADPH oxidase isoform 2); SIGLEC7 (Sialic acid-binding immunoglobulin-type lectin 7), also called CD328; SIGLEC9 (Sialic acid-binding immunoglobulin-type lectin 9), also called CD329; OX40 (Tumor necrosis factor receptor superfamily, member 4) also called CD134; and TIGIT. In some embodiments, the immune checkpoint protein is selected from PD-1, PD-L1 and PD-L2. In some embodiments, the immune checkpoint protein is selected from PD-1 and PD-L1. In some embodiments, the immune checkpoint protein is CTLA-4. In some embodiments, the immune checkpoint protein is PD-1. In some embodiments, immune checkpoint blockade comprises an anti-PD-1/PD-L1/PD-L2 immunotherapy. In some embodiments, immune checkpoint blockade comprises an anti-PD-1 immunotherapy. In some embodiments, immune checkpoint blockade comprises an anti-PD-1 and/or anti-PD-L1 immunotherapy. In some embodiments, immune checkpoint blockade comprises an anti-CTLA-4 immunotherapy. In some embodiments, immune checkpoint blockade comprises an anti-PD-1 and/or anti-PD-L1 immunotherapy and an anti-CTLA-4 immunotherapy. In some embodiments, the immunotherapy is anti-PD-1/PD-L1 immunotherapy. In some embodiments, the immunotherapy is anti-PD-1/PD-L1 axis immunotherapy. In some embodiments, immune checkpoint blockade comprises an anti-LAG-3. In some embodiments, immune checkpoint blockade comprises an anti-PD-1 and/or anti-PD-L1 immunotherapy and an anti-LAG-3 immunotherapy.

In some embodiments, the resistance-associated factor is determined by a method comprising:
a. receiving expression levels for a plurality of factors
   i. in a population of subjects known to respond to the therapy (responders);
   ii. in a population of subjects known to not respond to the therapy (non-responders); and
   iii. in the subject;
b. calculate for at least one factor of the plurality of factors a resistance score; and
c. classify a factor with a resistance score beyond a threshold as a resistance-associated factor.

In some embodiments, resistance-associated factors are in each subject. In some embodiments, resistance-associated factors are in the responders. In some embodiments, resistance-associated factors are in the non-responders. In some embodiments, the resistance-associated factors are labeled with the labels. In some embodiments, the expression levels of the resistance-associated factors are labeled with the labels. In some embodiments, the resistance-associated factors are resistance-associated proteins.

In some embodiments, the immunotherapy is a blocking antibody. In some embodiments, the immunotherapy is administration of a blocking antibody to the subject.

In some embodiments, the ICI is a monoclonal antibody (mAb) against PD-1 or PD-L1. In some embodiments, the ICI is a mAb that neutralizes/blocks/inhibits/modulates the PD-1 pathway. In some embodiments, the ICI is a mAb against PD-1. In some embodiments, the anti-PD-1 mAb is Pembrolizumab (Keytruda; formerly called lambrolizumab). In some embodiments, the anti-PD-1 mAb is Nivolumab (Opdivo). In some embodiments, the anti-PD-1 mAb is Pidilizumab (CT0011). In some embodiments, the anti-PD-1 mAb is Cemiplimab (Libtayo, REGN2810). In some embodiments, the anti-PD-1 mAb is any one of AMP-224, MEDI0680, or PDR001. In some embodiments, the ICI is a mAb against PD-L1. In some embodiments, the anti-PD-L1 mAb is selected from Atezolizumab (Tecentriq), Avelumab (Bavencio), and Durvalumab (Imfinzi). In some embodiments, the anti-PD-L1 mAb is Atezolizumab. In some embodiments, the anti-PD-L1 mAb is Durvalumab. In some embodiments, the ICI is a mAb against CTLA-4. In some embodiments, the anti-CTLA-4 mAb is ipilimumab. In some embodiments, the ICI is a mAb against LAG-3. In some embodiments, the anti-LAG-3 mAb is Relatlimab.

As used herein, the term “factor” refers to any measurable biological molecule produced by the subject. In some embodiments, the factor is a protein. In some embodiments, the factor is an RNA. In some embodiments, the factor is a gene. In some embodiments, the factor is a secreted factor. In some embodiments, the secreted factors are selected from cytokines, chemokines, growth factors, soluble receptors and enzymes. In some embodiments, the factor is a soluble factor. In some embodiment, the factor is cellular factor. In some embodiments, the factor is membranal factor. In some embodiments, the factor is a cell adhesion molecule. In some embodiments, the factor is a factor found in blood. In some embodiments, the factor is a host-generated factor. In some embodiments, the factor is a resistance factor.

In some embodiments, the expression is protein expression. In some embodiments, the expression is secreted protein expression. In some embodiments, protein expression is soluble protein expression. In some embodiment, the expression is cellular protein expression. In some embodiments, the expression is membranal protein expression. In some embodiments, the expression is mRNA expression. In some embodiments, the expression is protein expression or mRNA expression. In some embodiments, expression level is concentration. In some embodiments, concentration is concentration level. It will be understood by a skilled artisan that when the presence of factor is measured in a liquid sample the expression can be provided as a concentration such as mg/ml or in arbitrary units according to the method of determining the factor's expression. Arbitrary units can be selected from relative fluorescence unit (RFU) and Normalized Protein expression (NPX), or any other arbitrary units used as measurement of expression. The terms “expression” and “expression levels” are used herein interchangeably and refer to the amount of a gene product present in the sample. In some embodiments, gene product includes polynucleotide, e.g., tumor DNA, circulating tumor DNA, or circulating DNA. In some embodiments, the DNA is cell-free DNA. In some embodiments, determining comprises quantification of expression levels. In some embodiments, determining comprises normalization of expression levels. Determining of the expression level of the factor can be performed by any method known in the art. Methods of determining protein expression include, for example, antibody arrays, immunoblotting, immunohistochemistry, flow cytometry (FACS), ELISA, proximity extension assay (PEA), aptamer-based assays, proteomics arrays, proteome sequencing, flow cytometry (CyTOF), multiplex assays, mass spectrometry and chromatography. In some embodiments, determining protein expression levels comprises ELISA. In some embodiments, determining protein expression levels comprises protein array hybridization. In some embodiments, determining protein expression levels comprises mass-spectrometry quantification. In some embodiments, determining protein expression levels comprises PEA. In some embodiments, determining protein expression levels comprises aptamers. Methods of determining mRNA expression include, for example, RT-PCR, quantitative PCR, real-time PCR, microarrays, northern blotting, in situ hybridization, next generation sequencing, and massively parallel sequencing.

In some embodiments, the receiving factor expression levels is providing factor expression levels. In some embodiments, the receiving factor expression levels is determining factor expression levels. In some embodiments, determining is measuring. In some embodiments, the measuring is in a sample. In some embodiments, the expression levels were detected in a sample. In some embodiments, the sample is a biological sample. In some embodiments, the sample is provided by the subjects. In some embodiments, the sample is provided by the subject. In some embodiments, the sample is provided by a responder. In some embodiments, the sample is provided by a non-responder. In some embodiments, each subject of the population of responders provided a sample. In some embodiments, each subject of the population of non-responders provided a sample. In some embodiments, the sample is provided by a subject before receiving the therapy. In some embodiments, the factor expression level is from a time point before administration of the therapy. In some embodiments, the therapy is a monotherapy. In some embodiments, the therapy is an anti-PD-1/PD-L1 immunotherapy. In some embodiments, the therapy is a combination therapy. In some embodiments, the therapy is an anti-PD-1/PD-L1 immunotherapy and chemotherapy. In some embodiments, the sample is provided by a subject after receiving the therapy.

In some embodiments, the determining is directly in the sample. In some embodiments, the determining is in the unprocessed sample. In some embodiments, the determining is in a processed sample. In some embodiments, the method further comprises processing the sample. In some embodiments, processing comprises isolating proteins from the sample. In some embodiments, processing comprises isolating nucleic acids from the sample. In some embodiments, the nucleic acid is RNA. In some embodiments, the RNA is mRNA. In some embodiments, the processing comprises lysing cells in the sample. In some embodiments, the nucleic acid is cell free DNA. In some embodiments, the nucleic acid is tumor cell DNA.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the sample is a biological sample. In some embodiments, the sample is tissue. In some embodiments, the tissue sample is tumor sample. In some embodiments, the sample is a fluid. In some embodiments, the fluid is a biological fluid. In some embodiments, the sample is from the subject. In some embodiments, the sample is not a tumor sample. In some embodiments, the sample is a tumor sample. In some embodiments, the sample is not a hematopoietic cancer and the sample is a blood sample. In some embodiments, the sample is a sample that does not comprise cancer cells. In some embodiments, a blood sample comprises a peripheral blood sample, serum sample and a plasma sample. In some embodiments, the sample is a plasma sample. In some embodiments, the sample is a serum sample. In some embodiments, processing comprises isolating plasma. In some embodiments, processing comprises isolating serum. In some embodiments, the biological fluid is selected from, blood, plasma, serum, lymph, cerebral spinal fluid, urine, feces, semen, tumor fluid and gastric fluid. In some embodiments, the sample obtained from the subject and the responders are the same type of sample. In some embodiments, the sample obtained from the subject and the responders are different types of samples. In some embodiments, the sample obtained from the subject and the non-responders are the same type of sample. In some embodiments, the sample obtained from the subject and the non-responders are different types of samples. In some embodiments, the sample obtained from the non-responders and the responders are the same type of sample. In some embodiments, the sample obtained from the non-responders and the responders are different types of samples. In some embodiments, the sample obtained from the subject, the non-responders and the responders are the same type of sample. In some embodiments, the sample obtained from the subject, the non-responders and the responders are blood samples. In some embodiments, the sample obtained from the subject, the non-responders and the responders are plasma samples. In some embodiments, the sample obtained from the subject, the non-responders and the responders are serum samples. In some embodiments, the sample obtained from the subject, the non-responders and the responders are different types of samples.

In some embodiments, a factor is a factor of the plurality of factors. In some embodiments, expression levels of a plurality of factors are received. In some embodiments, expression levels of at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10000, 12000, 15000, 20000, 25000, 30000, 35000, or 40000 factors is received. Each possibility represents a separate embodiment of the invention. In some embodiments, expression levels of at least 50 factors are received. In some embodiments, expression levels of at least 100 factors are received. In some embodiments, expression levels of at least 200 factors are received. In some embodiments, expression levels of at least 300 factors are received. In some embodiments, expression levels of at least 350 factors are received. In some embodiments, expression levels of at least 375 factors are received. In some embodiments, expression levels of at least 380 factors are received. In some embodiments, expression levels of at least 385 factors are received. In some embodiments, expression levels of at least 388 factors are received. In some embodiments, a plurality is at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10000, 12000, 15000, 20000, 25000, 30000, 35000, or 40000. Each possibility represents a separate embodiment of the invention. In some embodiments, a plurality is at least 50 factors. In some embodiments, a plurality is at least 100 factors. In some embodiments, a plurality is at least 200 factors. In some embodiments, a plurality is at least 300 factors. In some embodiments, a plurality is at least 350 factors. In some embodiments, a plurality is at least 375 factors. In some embodiments, a plurality is at least 380 factors. In some embodiments, a plurality is at least 385 factors. In some embodiments, a plurality is at least 388 factors. In some embodiments, expression levels of at least 50 factors are received. In some embodiments, expression levels of at least 100 factors are received. In some embodiments, expression levels of at least 200 factors are received. In some embodiments, expression levels of at least 300 factors are received. In some embodiments, expression levels of at least 350 factors are received. In some embodiments, expression levels of at least 375 factors are received. In some embodiments, expression levels of at least 380 factors are received. In some embodiments, expression levels of at least 385 factors are received. In some embodiments, expression levels of at least 388 factors are received. In some embodiments, expression levels of at least 400 factors are received. In some embodiments, expression levels of at least 1000 factor are received. In some embodiments, expression levels of at least 5000 factors are received. In some embodiments, expression levels of at least 6000 factors are received. In some embodiments, expression levels of at least 7000 factors are received. In some embodiments, expression levels of at least 8000 factors are received.

In some embodiments, the factor is selected from a factor provided in Table 4. In some embodiments, the plurality of factors is selected from the factors provided in Table 4. In some embodiments, the plurality of factors comprises at least two factors selected from those provided in Table 4. In some embodiments, the plurality of factors consists of factors selected from Table 4. In some embodiments, the factor provided in Table 4 are: KCNAB2, IL12B, IL23A, MCL1, KIR2DS2, AGA, RPN1, LAT, MFAP2, PUF60, MPZ, ACE, RNF122, TXNDC5, CDH15, FGFBP3, COL11A2, INPP5E, ADH7, MVK, RNF146, SOCS3, RBFOX2, ARFGAP1, SRSF6, RBM23, DDR1, APOF, TRA2B, MCTS1, TBCA, RGS7, PTPN9, CSNKIG2, ILF3, TPPP2, ARHGEF2, SRSF7, EWSR1, FSTL1, SPP1, FLRT2, FLRT3, VTN, ATPIB1, WFIKKN2, NRAC, PKD2, HSPA9, EMC4, ASAP2, NAPIL2, HTR7, DCUNID3, RBL2, MADIL1, GRB14, RBBP5, NAB2, CSFIR, CCN4, GPD1, KLK3, CXCL13, GZMA, C9, IL12B, RAP1GAP, IGFBP1, DHX58, COPS2, IL1RAP, CCL25, HPX, ADM, CD93, ISG15, MYL6B, HSPAIA, MBD1, TRAPPC3, AKT2, CRLF1, FTL, RBBP4, BMPER, SERPINB5, PMP2, OTC, OTOR, AOC1, FGFBP1, ATRN, NAGLU, SAA1, SAA4, CLSTN1, GSS, DLD, EPHB4, PRSS27, MUC16, CFHR2, HTRA1, KRT19, RBP4, SMOC2, BTD, TXLNA, MZB1, FADD, GSN, CDH17, LECT2, ADAMTSL1, RNASET2, SEMA4A, DDOST, BDH2, SNRPB2, GOLM1, RAB3A, CD46, SEPTIN6, WWOX, WDR5, HPCAL1, ALDH5A1, VAT1, SARS1, AFM, CDA, ITLN1, LRIG1, GREM1, PTGR2, UBE2L6, CLTA, GSR, PDCD6, SNCG, CRH, RGS21, UBE2R2, BASP1, GBP5, LMNB2, POP7, RAETIL, SEMA5B, CNTN3, UBL3, MMACHC, GTF2B, GCHFR, LRATD2, SGK1, TSEN15, SAR1B, CDK5RAP3, HAUS1, NKIRAS1, PHOSPHO2, PCDH17, TRIM5, ALDH7A1, TXNL4A, CEP20, PDE1B, ITGA4, ITGB1, LRFN3, ADGRB1, SGSH, MGAT5, B3GAT1, MGAT5, FBLN7, APBB1IP, PON2, PPP2R5D, RBFOX1, TIMP1, GEMIN7, CSNK1A1L, PHF11, BTN2A2, SKP2, SPATA46, LIN7A, BORCS5, ARRDC5, PCYT1A, PHYH, ANKRD63, VCX, NTAN1, STARD7, APOL2, FLT4, RCSD1, INIP, VMAC, XPNPEP3, IFNE, NELFA, KDM8, NCBP1, USF2, LRRC75A, APCS, PLCD1, ESPN, RFX5, RPS6KB2, NOMO2, TCEAL2, CES3, DYRK1A, CYP2C19, CF1, IGFBP3, IL6, LEP, CRTC3, VEGFA, IL1RAP, HGF, PLA2G2A, CCL25, SERPINA7, POR, CCN3, HPX, IGFBP1, MMP3, FGA, FGB, FGG, BCAM, SPINT1, HAT1, GHR, CFP, CNTN1, SERPINF2, IL19, MB, C9, IGHM, LBP, NAAA, HAPLN1, IDS, NID1, ACAN, TGFB1, DLL4, FCGR3B, ACY1, IBSP, SERPINA4, POSTN, SELE, B2M, HAMP, SERPINA1, AHSG, CKB, CKM, PROC, PROC, ANGPTL4, MBD4, PSMD7, IGHE, CXCL10, KLKB1, CFH, PFDN5, RBM39, DCTPP1, PRSS22, KYNU, IL6, AFM, SERPINA6, ITIH4, SFN, CCL7, LYZ, MMP13, STC1, CAPG, PI3, GPC5, HRG, SCGB2A1, SIRT2, TNFAIP6, CD300C, GPNMB, KRT18, TNFSF14, LEPR, PRKCG, FGL1, PGLYRP2, NPFF, MFAP4, TMX3, PRKCSH, DEFB112, SEMA4D, ACP6, AFP, NGF, FTH1, FTL, DMKN, EPHA10, CHRDL2, TP53, AOC1, IFNA8, CSH1, CSH2, TNC, PLTP, CCN1, CLSTN3, OIT3, GGT2, FMOD, C5orf38, VWA1, INHBC, ADGRF5, CIQL2, PCYOX1, AOC2, CFHR4, LRRC15, POSTN, UBE2J1, GFRAL, IGF2, LILRB5, LILRA6, APOA2, VWA2, DEPP1, CIQTNF3, SERPINA9, CFHR5, DLG3, GLTPD2, HBQ1, ENTPD1, AGGF1, NRG2, SPON2, FAM241B, JAML, BCHE, GPNMB, APOD, DLL1, PEAR1, RSPO4, LEP, ARL8B, PCDH10, MFAP3L, CD14, COL15A1, PCDH10, HAVCR1, ARHGEF10, MANIA2, CRYZL1, TFPI2, PLXDC1, ACP2, BTD, MFAP2, ITIH2, EFCAB14, PLA1A, GZMK, YBX1, IDO1, NQO1, SPOCK3, and NXT1.

The amino acid sequences of these factors can be found in the Uniprot database, for example, and each factor's Uniprot accession number is provided in Table 4. Further, methods, reagents, and assays for measuring expression levels of these factors are well known in the art and are commercially available.

In some embodiments, the population of responders suffers from the disease. In some embodiments, the disease is a proliferative disease. In some embodiments, the disease is cancer. In some embodiments, the responders all have the same disease. In some embodiments, the population of non-responders suffers from the disease. In some embodiments, the non-responders all suffer from the same disease. In some embodiments, the population of responders and non-responders all suffer from the same disease. In some embodiments, the population of responders and the subject suffer from the same disease. In some embodiments, the population of non-responders and the subject suffer from the same disease. In some embodiments, the population of non-responders, the population of responders and the subject suffer from the same disease.

In some embodiments, the expression levels are from the subject before receiving the therapy. In some embodiments, the expression levels are determined for the subject before receiving the therapy. In some embodiments, the expression levels are from time TO. In some embodiments, the expression levels are baseline expression levels. In some embodiments, the sample is provided by the subject before receiving the therapy. In some embodiments, the expression levels are from the subject before receiving a first treatment of the therapy. In some embodiments, the expression levels are from the subject before receiving the first cycle of the therapy. In some embodiments, a treatment is a dose. In some embodiments, a treatment is a regimen. In some embodiments, a treatment is a combination of dose and regimen.

In some embodiments, before is at least 1 hour, 2 hours, 3 hours, 6 hours, 8 hours, 12 hours, 1 day, 2 days, 3 days, 5 days, 1 week, 2 weeks, 3 weeks, 4 weeks, 1 month, 2 months, 3 months, 4 months, 5 months, or 6 months before the therapy or before administration of the therapy. Each possibility represents a separate embodiment of the invention. In some embodiments, before is at least 1 hour before. In some embodiments, before is just before the therapy or before administration of the therapy. In some embodiments, before is at most 1 hour, 2 hours, 3 hours, 4 hours, 6 hours, 9 hours, 12 hours, 18 hours, 24 hours, 2 days, 3 days, 5 days, 1 week, 2 weeks, 3 weeks, 4 weeks, 1 month, 2 months, 3 months, 4 months, 5 months, or 6 months before the therapy or before administration of the therapy. Each possibility represents a separate embodiment of the invention. In some embodiments, before is at most 24 hours before the therapy or before administration of the therapy. In some embodiments, administration of the therapy is the first administration of the therapy. In some embodiments, administration of the therapy is any administration of the therapy.

In some embodiments, the expression levels are from the subject after receiving the therapy. In some embodiments, the expression levels are from time T1. In some embodiments, the sample is provided by the subject after receiving the therapy. In some embodiments, the expression levels are from the subject after receiving a first treatment of the therapy. In some embodiments, the expression levels are from the subject after receiving any treatment with the therapy.

In some embodiments, after is at a time after initiation of the therapy, or after administration of the therapy, sufficient for altered expression of the at least one factor. In some embodiments, after is at a time after initiation of the therapy, or after administration of the first treatment of the therapy. In some embodiments, after is at least 1 day, 2 days, 3 days, 4 days, 5 days, 6 days, 1 week, 2 weeks, 3 weeks, 4 weeks, 6 weeks, 1 month, 2 months, 3 months, 4 months, 5 months, 6 months, or a year after. Each possibility represents a separate embodiment of the invention. In some embodiments, after is at least 24 hours after. In some embodiments, after is at least 2 weeks after. In some embodiments, after is at least 3 weeks after. In some embodiments, after is at least 6 weeks after. In some embodiments, after is at most 1 week, 2 weeks, 3 weeks, 4 weeks, 6 weeks, 1 month, 2 months, 3 months, 4 months, 5 months, 6 months or a year after initiation of the therapy, or after administration of the therapy. Each possibility represents a separate embodiment of the invention.

In some embodiments, the receiving expression levels comprises receiving factor expression levels for a group of factors larger than the plurality of factors. In some embodiments, the received expression levels for the larger group are received for responders and non-responders. In some embodiments, a subgroup of proteins is selected from the group. In some embodiments, a subgroup is a subset. In some embodiments, the subgroup is designated the plurality of factors. In some embodiments, the method comprises designating. In some embodiments, the receiving further comprises for each factor of the group applying a machine learning algorithm. In some embodiments, the algorithm classifies factors as from responders and non-responders. In some embodiments, the algorithm outputs if a subject that provided the sample that had the measured factor expression level is a responder or non-responder. In some embodiments, the receiving further comprises selecting a subgroup of factors for which the algorithm most evenly divides the subjects into responders and non-responders. In some embodiments, the subjects are all the subjects in the populations of responders and non-responders. In some embodiments, the factors are processed with an algorithm that most evenly divides all subjects, responders and non-responders, into groups of responders and non-responders (even if designations are incorrect) are selected as the subgroup. In some embodiments, the algorithm is trained on the received factor expression levels in responders and non-responders. In some embodiments, the algorithm is trained on a training set. In some embodiments, training is on expression levels and tags indicating if an expression level was from a responder or non-responder. In some embodiments, training is on expression levels, clinical information and tags indicating if an expression level was from a responder or non-responder. In some embodiments, training is on the number of the resistance associated factors. In some embodiments, training is on the number of the resistance associated factors and tags indicating if a number of resistance associate factors was from a responder or non-responder.

In some embodiments, the receiving further comprises for each factor of the group determining the average difference between responder and non-responders. In some embodiments, the receiving further comprises for each factor of the group determining the statistical significance between the levels in responders and non-responders. In some embodiments, the statistical significance is between the averages. In some embodiments, the statistical significance is the p-value. In some embodiments, the receiving further comprises selecting a subgroup of factors with the greatest statistical significance. In some embodiments, a statistical test is applied to determine significance. In some embodiments, the test is a Kolmogorov-Smirnov test. In some embodiments, the subgroup comprises a predetermined number of factors with the greatest significance. In some embodiments, the predetermined number is about 50 factors. In some embodiments, the predetermined number is at least 50 factors.

In some embodiments, the subgroup comprises the factors whose algorithm most evenly divides the subjects. In some embodiments, evenly divides is into responders and non-responders. In some embodiments, the subgroup is the top 50, 100, 150, 200, 250, 300, 350, 400, 450, 500, 600, 700, 750, 800, 900, 1000, 1100, 1200, 1300, 1400, 1500, 1600, 1700, 1800, 1900, 2000, 3000, 4000, or 5000. Each possibility represents a separate embodiment of the invention. In some embodiments, the subgroup is the top 50. In some embodiments, the subgroup is the top 100. In some embodiments, the subgroup is the top 200. In some embodiments, the subgroup is the top 500.

In some embodiments, the method further comprises performing a dimensionality reduction step. In some embodiments, the reduction is with respect to the plurality of factors. In some embodiments, the reduction is reducing the number of factors in the plurality. In some embodiments, the dimensionality reduction step identifies a subgroup or a subset of factors. In some embodiments, factors are principal factors. In some embodiments, the training set comprises only the expression levels of the subset/subgroup of factors. In some embodiments, the subgroup or subset of factors are the factors that most evenly balance the predicted number of responders and non-responders. In some embodiments, predicted is predicted by the machine learning algorithm. In some embodiments, the machine learning algorithm is the trained machine learning algorithm. In some embodiments, the machine learning algorithm is the machine learning algorithm during training.

In some embodiments, a preprocessing stage may take place to preprocess the received expression levels. In some embodiments, the preprocessing stage may comprise at least one of data cleaning and normalizing, feature selection, feature extraction, dimensionality reduction, and/or any other suitable preprocessing method or technique. Feature selection can be performed by statistical tests, such as the Kolmogorov Smirnov (KS) test, or any other test known in the art.

In some embodiments, factor selection and/or dimensionality reduction steps may be performed, to reduce the number of factors in each sample and/or to obtain a set of principal factors, e.g., those factors that may have significant predictive power. In some embodiments, factor selection is RAP selection. Accordingly, in some embodiments, a factor selection and/or dimensionality reduction step may result in a reduction of the number of factors in each sample and/or set of values. In some embodiments, dimensionality reduction selects principal factors, e.g., proteins, based on the level of response predictive power a factor generates with respect to the desired prediction. In specific embodiments, the dimensionality reduction involves regarding all or some factors as vector components and calculating their norm.

In some embodiments, any suitable factor selection and/or dimensionality reduction method or technique may be employed, such as, but not limited to:
ANOVA with S₀ parameter: Analysis of variance with an additional parameter (S₀) that controls for the relative importance of features based on resulted test p-values and difference between the group means (see, e.g., Tusher, Tibshirani and Chu, PNAS 98, pp 5116-21, 2001).
Scalable EMpirical Bayes Model Selection (SEMMS): An empirical Bayes feature selection method which applies a parsimonious mixture model to identify significant predictors (see, e.g., Bar, Booth, and Wells. A scalable empirical Bayes approach to variable selection in generalized linear models, 2019).
L2N: A method for differential expression analysis that uses a three-component mixture model. The model consists of two log-normal components (L2) for differentially expressed features, one component for under-expressed features and the other for overexpressed features, and a single normal component (N) for non-differentially expressed features (see, e.g., Bar and Schifano. Differential variation and expression analysis. Stat 8, e237, doi: 10.1002/sta4.237, 2019).
Genetic algorithms: A family of heuristic optimization algorithms that employ organic evolutionary techniques such as random mutations, recombination, and natural selection as methods for achieving optimal configurations (see, e.g., Popovic, Sifrim, Pavlopoulos, Moreau, and Bart De Moor. A Simple Genetic Algorithm for Biomarker Mining. 2012).
Naïve classifier: The naïve classifier evaluates a response score by reducing the dimension to a single score. This is performed by regarding all features (e.g., specific profiles such as protein expression levels) as component of a vector and calculating its norm. The dimension reduction reduces the possible risk of an over-fitting. In some embodiments, the vector components are normalized according to the typical component value among patients that belong to the same response group (e.g., responders), such that the normalized norm quantifies the amount of deviation from the typical respective class value. In additional embodiments, the naïve classifier enables training using data of subjects that belong only to part of the response groups.

As used herein, the term “responder” or a subject “known to respond” are used interchangeably and refer to a subject that when administered a therapy displays an improvement in at least one criteria of the disease being treated by the therapy or does not show an increase in severity of the disease. In some embodiments, a responder is a subject that when administered a therapy displays an improvement in the disease that is being treated by the therapy. In some embodiments, a responder is a subject that when administered a therapy displays a clinical benefit. In some embodiments, a responder is a subject that when administered a therapy does not show an increase in severity of the disease. In some embodiments, an increase in severity is over time. In some embodiments, does not show an increase in severity is stable disease. In some embodiments, a responder is a subject that when administered a therapy show mixed response. In some embodiments, a responder is a subject that when administered a therapy show mixed response, wherein mixed response is improvement in at least one criteria of the disease but does not show an improvement in other criteria of the disease. In some embodiments, mixed response is shrinkage of some lesions in combination with growth of new or existing lesions. In some embodiments, a responder is a subject for which the therapy produces an anti-disease response. In some embodiments, for a subject with cancer, a responder is a subject in which the therapy produces an anticancer response. In some embodiments, a response is not a reduction in side effects. In some embodiments, a response is a reduction in side effects. In some embodiments, a response is a response against the disease itself. In some embodiments, an anticancer response is an antitumor response. In some embodiments, an antitumor response comprises tumor regression. In some embodiments, an antitumor response comprises tumor shrinkage. In some embodiments, an antitumor response comprises a lack of tumor growth. In some embodiments, an antitumor response comprises a lack of tumor metastasis. In some embodiments, an antitumor response comprises a lack of tumor hyperproliferation. In some embodiments, an improvement is in at least one symptom of the disease. In some embodiments, response is complete response. In some embodiments, response is minimal response. In some embodiments, response is partial response. In some embodiments, response comprises stable disease. In some embodiments, responder is a subject with a favorable response to the therapy. In some embodiments, non-responder is a subject with a non-favorable response to the therapy. In some embodiments, a non-favorable response is an increase in tumor burden. Increases in tumor burden can encompass any increase in tumor size or total cancer cell number such as increase in tumor size, increase in tumor spread, increase in metastasis, increase in tumor cell proliferation or any other increase. In some embodiments, response is response to a monotherapy. In some embodiments, response is response to a combination therapy.

As used herein, a “favorable response” of the cancer patient indicates “responsiveness” of the cancer patient to the treatment with the therapy, namely, the treatment of the responsive cancer patient with the therapy will lead to the desired clinical outcome such as tumor regression, tumor shrinkage or tumor necrosis; reduction in tumor burden; an anti-tumor response by the immune system; preventing or delaying tumor recurrence, tumor growth or tumor metastasis. In some embodiments, the subject is complete responder or treatment with the cancer therapy leads to stable disease. In some embodiments, a complete responder is a subject in which there is an absence of detectable cancer after treatment with the therapy. In this case, it is possible and advised to continue the treatment of the responsive cancer patient with the therapy or if the patient is cancer free to discontinue treatment. In some embodiments, the method further comprises continuing to administer the therapy to a subject that is not a non-responder. In some embodiments, the subject is non-responder, a minimal responder, partial responder or has a stable disease, and the method further comprises continuing to administer the therapy to a subject, as well as treating the subject with an additional therapy (e.g., determined using the resistance associated protein (RAP) analysis provided herein) to increase responsiveness. In some embodiments, a subject that is not a non-responder is a responder.

As used herein, the term “non-responder” and a subject “known to not respond” are used interchangeably and refer to a subject that when administered a therapy displays no improvement or stabilization in disease. In some embodiments, a non-responder displays a worsening of disease when administered a therapy. In some embodiments, a non-responder is a subject that when administered a therapy displays no clinical benefit. In some embodiments, non-responder is not a subject that experiences a side effect of the therapy. In some embodiments, a non-responder is a subject in which the disease progresses. In some embodiments, a non-responder is a subject in which the disease does not stabilize after therapy. In some embodiments, a non-responder is a subject in which the disease does not improve after therapy. In some embodiments, a non-responder is a subject that is not a responder as defined hereinabove. In some embodiments, a non-responder is a subject with a non-favorable response to the therapy. In some embodiments, a non-responder is a subject resistant to the therapy. In some embodiments, a non-responder is a subject refractory to the therapy. In some embodiments, non-response is non-response to a monotherapy. In some embodiments, non-response is non-response to a combination therapy.

As used herein a “non-favorable response” of the cancer patient indicates “non-responsiveness” of the cancer patient to the treatment with the therapy and thus the treatment of the non-responsive cancer patient with the therapy will not lead to the desired clinical outcome, and potentially to a non-desired outcomes such as tumor expansion, recurrence, or metastases. In some embodiments, the method further comprises discontinuing administration of the therapy to a subject that is a non-responder. In some embodiments the method further comprises continuing to administer the therapy to a subject, in combination with an additional therapy. In some embodiments, the additional therapy increases responsiveness of a non-responsive patient.

In some embodiments, the method is for determining whether the response is considered a durable response (e.g., a progression-free survival of more than 6 months). In some embodiments, response is response for at least 3-months. In some embodiments, the response is response at a time from treatment. In some embodiments, from treatment is from the commencement of treatment. In some embodiments, response is response at 3-months. In some embodiments, response is response for at least 6-months. In some embodiments, response is response at 6-months. In some embodiments, response is response for at least 7-months. In some embodiments, response is response at 7-months. In some embodiments, response is response for at least 1-year. In some embodiments, response is response at 1-year. In some embodiments, response is response for at least 2-year. In some embodiments, response is response at 2-year. In some embodiments, response is response for at least 3-year. In some embodiments, response is response at 3-year. In some embodiments, response is response for at least 4-year. In some embodiments, response is response at 4-year. In some embodiments, response is response for at least 5-year. In some embodiments, response is response at 5-year. It will be understood by a skilled artisan that response for at least a given amount of time comprises at least monitoring response at that time point and also potentially monitoring response up until that time point.

In some embodiments, the method further comprises administering the therapy to the subject predicted to respond to the therapy. In some embodiments, the method further comprises continuing to administering the therapy to the subject predicted to respond to the therapy. In some embodiments, the method further comprises not administering the therapy to the subject predicted to not respond to the therapy. In some embodiments, the method further comprises discontinuing the therapy to the subject predicted to not respond to the therapy. In some embodiments, the method further comprises administering an alternative therapy to the subject predicted to be a non-responder. In some embodiments, the alternative therapy is an additional therapy. In some embodiments, the additional therapy is chemotherapy. In some embodiments, the method further comprises administering the therapy or continuing to administer the therapy in combination with an agent or therapy that blocks or inhibits at least one of the resistance-associated factors in the subject predicted to be resistant to the therapy. In some embodiments, an agent or therapy that blocks or inhibits at least one of the resistance-associated factors is an additional therapy. In some embodiments, an agent or therapy that blocks or inhibits the signaling pathway of at least one of the resistance-associated factors is an additional therapy. In some embodiments, the combination therapy is administered to a subject predicted to be a non-responder.

In some embodiments, the method further comprises administering the monotherapy to a subject predicted to respond to the monotherapy. In some embodiments, the method further comprises administering the monotherapy to a subject with PD-L1 high cancer predicted to respond to the monotherapy. In some embodiments, the method further comprises administering a combination therapy to a subject predicted to not respond to the monotherapy. In some embodiments, the method further comprises administering the combination therapy to a subject with PD-L1 high cancer predicted to not respond to the monotherapy.

In some embodiments, the method further comprises administering the combination therapy to a subject predicted to respond to the combination therapy. In some embodiments, the method further comprises administering the combination therapy to a subject with PD-L1 low or negative cancer predicted to respond to the combination therapy. In some embodiments, the method further comprises administering an alternative therapy to a subject predicted to not respond to the combination therapy. In some embodiments, the method further comprises administering an alternative therapy to a subject with PD-L1 low or negative cancer predicted to not respond to the combination therapy. Examples of alternative therapies include, but are not limited to other ICI combination (e.g., with anti-CTLA-4) and non-chemotherapeutic treatments.

In some embodiments, the method further comprises administering to the subject (e.g., a non-responder) an agent that modulates the at least one factor. In some embodiments, modulates comprises inhibits, blocks and regulates. In some embodiments, modulates is inhibits. In some embodiments, the method further comprises administering to the subject (e.g., a non-responder) an agent that modulates a pathway that comprises the at least one factor. In some embodiments, modulating the at least one factor is modulating a pathway comprising the at least one factor. In some embodiments, modulating a pathway comprising modulating a driver protein/gene that controls the at least one factor. In some embodiments, modulating a pathway comprising modulating a driver protein/gene that controls the pathway. In some embodiments, modulating a pathway comprising the at least one factor is modulating a receptor of the factor (e.g., using a receptor agonist or antagonists), a ligand or the factor, a paralog of the factor, or a combination thereof. In some embodiments, the modulating is modulating a plurality of factors. In some embodiments, the modulating is modulating a plurality of factors in the signature. In some embodiments, the modulation is modulating each factor in the signature. In some embodiments, the modulation achieves better response to therapy. In some embodiments the factor is a resistance-associated factor.

In some embodiments, a resistance score is a RAP score. In some embodiments, a resistance score is a response score. In some embodiments, a resistance score is 1-response score. In some embodiments, a resistance score is 10-response score. In some embodiments, response score is 1-resistance score. In some embodiments, response score is 10-resistance score. It will be understood by a skilled artisan that the response score and resistance score are inverses. Thus, if the scale of the scores is 0-1 then the conversion of one score to the other is 1-score. Whereas if the scale of the scores is 0-10 then the conversion of one score to the other is 10-score. The same can be used for any scale being used for the two scores. In some embodiments, resistance score is total resistance score. In some embodiments, response score is total response score. In some embodiments, a RAP score is a total RAP score. In some embodiments, the resistance score is based on similarity of the factor expression level in the subject to the factor expression level in the non-responders. In some embodiments, the resistance score is based on similarity of the factor expression level in the subject to the factor expression level in the responders. In some embodiments, based on is calculated based on. In some embodiments, similarity is lack of similarity. In some embodiments, similarity to responders is lack of similarity to non-responders. In some embodiments, similarity to non-responders is lack of similarity to responders. In some embodiments, similarity is measured on a scale.

In some embodiments, the scale is from 0 to 1, wherein 1 is perfectly similar to non-responders and 0 is perfectly similar to responders. In some embodiments, the resistance score is from 0 to 1, wherein 1 is perfectly similar to non-responders and 0 is perfectly similar to responders. In some embodiments, the resistance score is based on similarity of the factor expression level in the subject to the factor expression level in the non-responders and the factor expression level in the responders. In some embodiments, the response score is from 0 to 1, wherein 1 is perfectly similar to responders and 0 is perfectly similar to non-responders. In some embodiments, the response score is the PROphet score. In some embodiments, a prophet positive subject is a subject with a response score above a predetermined threshold. In some embodiments, a prophet negative subject is a subject with a response score below a predetermined threshold. In some embodiments, the response score is based on similarity of the factor expression level in the subject to the factor expression level in the non-responders and the factor expression level in the responders. In some embodiments, a response score from 0.5 to 1 indicates the subject is a responder. In some embodiments, a response score above 0.5 indicates the subject is a responder. In some embodiments, a response score from 0.5 to 0 indicates the subject is a non-responder. In some embodiments, a response score below 0.5 indicates the subject is a non-responder.

In some embodiments, the scale is from 0 to 10, wherein 10 is perfectly similar to responders and 0 is perfectly similar to non-responders. In some embodiments, the resistance score is from 0 to 10, wherein 10 is perfectly similar to non-responders and 0 is perfectly similar to responders. In some embodiments, the resistance score is based on similarity of the factor expression level in the subject to the factor expression level in the non-responders and the factor expression level in the responders. In some embodiments, the response score is from 0 to 10, wherein 10 is perfectly similar to responders and 0 is perfectly similar to non-responders. In some embodiments, the response score is the PROphet score. In some embodiments, the response score is the total response score. In some embodiments, a prophet positive subject is a subject with a response score above a predetermined threshold. In some embodiments, a prophet negative subject is a subject with a response score below a predetermined threshold. In some embodiments, the response score is based on similarity of the factor expression level in the subject to the factor expression level in the non-responders and the factor expression level in the responders. In some embodiments, a response score from 5 to 10 indicates the subject is a responder. In some embodiments, a response score above 5 indicates the subject is a responder. In some embodiments, a response score from 5 to 0 indicates the subject is a non-responder. In some embodiments, a response score below 5 indicates the subject is a non-responder.

In some embodiments, the method comprises before step (b) selecting a subset of factors. In some embodiments, the subset is a subset of the plurality of factors. In some embodiments, before step (b) is before the calculating. In some embodiments, the subset is a subset of the plurality of factors. In some embodiments, the subset comprises the factors that best differentiate between the responders and non-responders. In some embodiments, the factors that best differentiate are the top percentage. In some embodiments, the top percentage is the top 1, 3, 5, 10, 15, 20, 25, 30, 35, 40, 45 or 50% of factors. Each possibility represents a separate embodiment of the invention. In some embodiments, the top percentage is the top 20%. In some embodiments, the top factors are the top 10, 20, 25, 30, 40, 50, 60, 70, 75, 80, 90 or 100 factors. Each possibility represents a separate embodiment of the invention. In some embodiments, the top factors are the top 50 factors. In some embodiments, selection comprises applying a Kolmogorov-Smirnov test. In some embodiments, the Kolmogorov-Smirnov test is applied to the received factor expression levels. In some embodiments, the Kolmogorov-Smirnov test determines how well a factor differentiates between responders and non-responders. In some embodiments, the Kolmogorov-Smirnov test outputs a measure of how well a factor differentiates and the best factors are the factors with the highest scores. In some embodiments, selection comprises applying an XGBoost algorithm. In some embodiments, the calculating is for the subset. In some embodiments, the calculating is for each factor of the subset.

In some embodiments, calculating comprises applying a machine learning algorithm. In some embodiments, calculating comprises applying a machine learning model. In some embodiments, the machine learning model is a machine learning algorithm. In some embodiments, the machine learning model implements a machine learning algorithm. In some embodiments, the algorithm is a classifier. In some embodiments, the algorithm is a regression model. In some embodiments, the algorithm is supervised. In some embodiments, the algorithm is unsupervised. In some embodiments, the machine learning algorithm is trained on the expression levels in responders. In some embodiments, the machine learning algorithm is trained on the expression levels in non-responders. In some embodiments, the machine learning algorithm is trained on the expression levels in responders and non-responders. In some embodiments, the machine learning algorithm is trained on a training set. In some embodiments, the machine learning algorithm is trained by a method of the invention. In some embodiments, a machine learning algorithm is applied to factors of the plurality of factors. In some embodiments, a machine learning algorithm is applied to each factor of the plurality of factors. In some embodiments, a machine learning algorithm is applied to the subset. In some embodiments, a machine learning algorithm is applied to the subset of factors. In some embodiments, a machine learning algorithm is applied to each factor of the subset of factors. In some embodiments, each factor is analyzed and calculated separately, and the machine learning algorithm does not use expression levels of more than one factor as the training set. In some embodiments, a trained machine learning algorithm is applied to individual protein expression levels from the subject. In some embodiments, a machine learning algorithm trained on expression levels of a specific factor in responders and non-responders is applied to the expression level of that specific factor in the subject. It will be understood by a skilled artisan, that for each of the factors of the plurality of factors, a different algorithm will be trained and then applied to each expression level of the subject. Thus, if three algorithms are separately trained on expression in responders and non-responders for Factor A, Factor B and Factor C, then the algorithm trained on Factor A expression levels will be applied to the subject's expression level of Factor A, the algorithm trained on Factor B expression levels will be applied to the subject's expression level of Factor B, and the algorithm trained on Factor C expression levels will be applied to the subject's expression level of Factor C. In some embodiments, during a training phase, the machine learning model is trained on a training set comprising expression data for a single factor from responders and non-responders, using corresponding annotations of “responder” or “non-responder” to predict or classify factor expression data according to classes “responder” and “non-responder”. In some embodiments, during an inference stage, the machine learning model is applied to expression data of the single factor from a subject to predict classification of the factor as similar to a responder or non-responder. In some embodiments, the classification is a resistance score. In some embodiments, the classification is a response score. In some embodiments, the classification is a measure of how similar the factor is to non-responders and dissimilar to responders.

In some embodiments, the trained machine learning algorithm is trained to predict responsiveness of subjects suffering from the disease to the therapy. In some embodiments, the trained machine learning algorithm is trained to output a resistance score. In some embodiments, the trained machine learning algorithm is trained to output a resistance probability. In some embodiments, the trained machine learning algorithm is trained to output clinical benefit probability. In some embodiments, the trained machine learning algorithm is trained to output an activity score. In some embodiments, the trained machine learning algorithm is trained to predict activity of a resistance-associated factor in a subject. In some embodiments, the trained machine learning algorithm is trained to predict if a factor is a resistance-associated factor in the subject. In some embodiments, the trained machine learning algorithm is trained to predict if a factor of the subject is a resistance-associated factor in the subject.

In some embodiments, the trained machine learning algorithm is trained to predict responsiveness of subjects suffering from the disease to the therapy. In some embodiments, the trained machine learning algorithm is trained to output a response score. In some embodiments, the trained machine learning algorithm is trained to output a response probability. In some embodiments, the trained machine learning algorithm is trained to output clinical benefit probability. In some embodiments, the trained machine learning algorithm is trained to output an activity score. In some embodiments, the trained machine learning algorithm is trained to predict activity of a response-associated factor in a subject. In some embodiments, the trained machine learning algorithm is trained to predict if a factor is a response-associated factor in the subject. In some embodiments, the trained machine learning algorithm is trained to predict if a factor of the subject is a response-associated factor in the subject.

In some embodiments, the training set comprises received factor expression levels. In some embodiments, the training set comprises received factor expression levels in both responders and non-responders. In some embodiments, the training set comprises received factor expression levels in both mono-responders and mono-non-responders. In some embodiments, the training set comprises received factor expression levels in both combo-responders and combo-non-responders. In some embodiments, the training set comprises received factor expression levels in mono-responders, mono-non-responders, combo-responders and combo-non-responders. In some embodiments, the training set comprises received factor expression levels for only one factor. In some embodiments, the training set comprises the number of resistance-associated factors or response-associated factors expressed in samples. In some embodiments, the sample are from subjects suffering from the disease. In some embodiments, the sample are from responders. In some embodiments, the sample are from non-responders. In some embodiments, the training set comprises at least one clinical parameter. In some embodiments, the clinical parameter is from subjects. In some embodiments, subjects are responders and non-responders. In some embodiments, the training set comprises labels. In some embodiments, the labels are associated with the responsiveness of the subjects. In some embodiments, the labels are responder or non-responder. In some embodiments, the resistance-associated factors are labeled with the labels. In some embodiments, the expression levels of the resistance-associated factors are labeled with the labels. In some embodiments, the at least one clinical parameter is labeled with the label.

According to some embodiments, the training set further comprises at least one clinical parameter of each responder and non-responder and the machine learning algorithm is applied to individual received factor expression levels from the subject and the subject's at least one clinical parameter. In some embodiments, the at least one clinical parameter is the sex of the subjects. In some embodiments, the training set further comprises the sex of the subjects. In some embodiments, the subjects are each subject. In some embodiments, sex is gender. In some embodiments, the at least one clinical parameter is sex. In some embodiments, sex is a subject's sex. In some embodiments, sex is male or female. In some embodiments, sex is sex at birth. In some embodiments, the training set comprises the sex of each responder. In some embodiments, the training set comprise the sex of each non-responder. In some embodiments, the training set comprises the sex of each mono-responder. In some embodiments, the training set comprise the sex of each mono-non-responder. In some embodiments, the training set comprises the sex of each combo-responder. In some embodiments, the training set comprise the sex of each combo-non-responder. In some embodiments, the clinical parameter is age. In some embodiments, age is a subject's age. In some embodiments, the clinical parameter is the line of treatment. In some embodiments, the line of treatment parameter is whether the therapy was a first line of treatment or an advanced treatment. In some embodiments, a line of treatment is first line treatment. In some embodiments, a line of treatment is a secondary treatment. In some embodiments, secondary treatment is an advanced treatment. It will be understood by a skilled artisan that advanced treatment may be any line of treatment after the first, e.g., second line, third line, fourth line, fifth line, etc. In some embodiments, the clinical parameter is whether the treatment is a first line treatment or an advanced treatment. In some embodiments, the clinical parameter is PD-L1 status. In some embodiments, PD-L1 status is PD-L1 status of the cancer. Methods of measuring PD-L1 levels in cancer cells (e.g., a tumor) are well known in the art and any such method may be employed. In some embodiments, PD-L1 status comprises high PD-L1 or low PD-L1. In some embodiments, PD-L1 status comprises high PD-L1, low PD-L1 or no PD-L1. In some embodiments, PD-L1 status comprises high PD-L1, medium PD-L1 or low PD-L1. In some embodiments, PD-L1 levels are numeric values between 0 to 100. In some embodiments, PD-L1 levels are percentages between 0 to 100. In some embodiments, PD-L1 status comprises PD-L1 expression in less than 1% of cancer cells, in 1-49% of cancer cells, or in 50% or more of cancer cells. In some embodiments, PD-L1 expression in less than 1% of cancer cells is no PD-L1 expression. In some embodiments, PD-L1 low or negative cancer comprises fewer than 50% of cancer cells being positive for PD-L1 expression. In some embodiments, expression is surface expression. In some embodiments, PD-L1 negative cancer comprises fewer than 1% of cancer cells being positive for PD-L1 expression. In some embodiments, PD-L1 expression in less than 1% of cancer cells is low PD-L1 expression. In some embodiments, PD-L1 expression in 1-49% of cancer cells is low PD-L1 expression. In some embodiments, PD-L1 low cancer comprises fewer than 1-49% of cancer cells being positive for PD-L1 expression. In some embodiments, PD-L1 expression in 1-49% of cancer cells is medium PD-L1 expression. In some embodiments, PD-L1 expression in 50% or more of cancer cells is high PD-L1 expression. In some embodiments, a high PD-L1 cancer comprises expression in at least 50% of cells. In some embodiments, PD-L1 high cancer comprises at least 50% of cancer cells being positive for PD-L1 expression. In some embodiments, a low PD-L1 cancer comprises expression in 1-49% of cells. In some embodiments, a no PD-L1 cancer comprises expression in 0% of cells. In some embodiments, a no PD-L1 cancer comprises expression in less than 1% of cells. In some embodiments, the PD-L1 low or negative cancer is PD-L1 low cancer. In some embodiments, the PD-L1 low or negative cancer is PD-L1 negative cancer. In some embodiments, a no PD-L1 cancer is a PD-L1 negative cancer.

In some embodiments, the clinical parameter is a known biomarker of the disease or mutations in known biomarkers of the disease. In some embodiments, the biomarker is selected from MYC, NOTCH, EGFR, HER2, BRAF, KRAS, MAP2K1, MET, NRAS, NTRK1, NTRK2, NTRK3, PIK3CA, RET, ROS1, TP53, ALK, CDKN2A, KIT, NFI, BFAST, FGFR, LDH, PTEN, RB1, PD-L1, MSI (Microsatelite Instability), TMB (Tumor Mutational Burden), or a combination thereof. In some embodiments, the clinical parameter is expression of the biomarker. In some embodiments, expression is percent expression. In some embodiments, expression is mutational status.

In some embodiments, the training set further comprises the sex, age and PD-L1 status of each responder and non-responder. In some embodiments, the training set further comprises the sex of each responder and non-responder. In some embodiments, the training set further comprises the age and PD-L1 status of each responder and non-responder. In some embodiments, the machine learning algorithm is applied to individual received factor expression levels from the subject and the subject's sex. In some embodiments, the machine learning algorithm is applied to individual received factor expression levels from the subject and the subject's sex, age and PD-L1 status. In some embodiments, the calculating comprises applying a machine learning algorithm trained on a training set comprising the received factor expression levels in responders and non-responders and at least one clinical parameter, to the expression levels from the subject and the subject's at least one clinical parameter and wherein the machine learning algorithm outputs the resistance score. In some embodiments, the training comprises the received factor expression levels in responders and non-responders and clinical parameters of each responder and non-responder and the machine learning algorithm is applied to individual received factor expression levels from the subject and the subject's clinical parameters and wherein the machine learning algorithm outputs response score. In some embodiments, the training comprises the received factor expression levels in responders and non-responders and a clinical parameter selected from sex, age and PD-L1 expression, or any combination thereof, of each responder and non-responder and the machine learning algorithm is applied to individual received factor expression levels from the subject and the subject's clinical parameters and wherein the machine learning algorithm outputs response prediction. In some embodiments, the training set comprises the number of resistance associated factors in each responder and non-responder and at least one clinical parameter and the machine learning algorithm is applied to the number of resistance associated factors from the subject and the subject's at least one clinical parameters and wherein the machine learning algorithm outputs a response prediction. In some embodiments, the training set comprises the number of resistance associated factors in each responder and non-responder and sex of each responder and non-responder and the machine learning algorithm is applied to the number of resistance associated factors from the subject and the subject's sex and wherein the machine learning algorithm outputs a response prediction. In some embodiments, the training set comprises the number of resistance associated factors in each responder and non-responder, age and PD-L1 status of each responder and non-responder and the machine learning algorithm is applied to the number of resistance associated factors from the subject and the subject's age and PD-L1 status and wherein the machine learning algorithm outputs a response prediction.

In some embodiments, the training set comprises the received factor expression levels in responder and non-responders. In some embodiments, the training set comprises the received factor expression levels in responder and non-responders and a clinical parameter. In some embodiments, the training set comprises the received factor expression levels in responder and non-responders and sex of each of the responders and non-responders. In some embodiments, the trained machine learning algorithm is applied to individual received factor expression levels from the subject. In some embodiments, the trained machine learning algorithm is applied to each received factor expression levels from the subject. In some embodiments, the trained machine learning algorithm is applied to individual received factor expression levels from the subject and a clinical parameter from the subject. In some embodiments, the trained machine learning algorithm is applied to individual received factor expression levels from the subject and the subject's sex.

In some embodiments, the clinical parameter is the type of treatment. In some embodiments, the clinical parameter is expression of a target of the therapy. In some embodiments, the clinical parameter is expression of a protein within a process that is a target of the therapy. In some embodiments, the process is a process comprising the target of the therapy. In some embodiments, expression is expression in the subject. In some embodiments, expression is expression in a diseased tissue. In some embodiments, expression is expression in a diseased tissue sample. In some embodiments, expression is expression in the tumor. In some embodiments, expression is expression in a tumor sample. In some embodiments, a tumor sample is a biopsy. In some embodiments, expression is expression not in the tumor. In some embodiments, expression is expression not in a tumor sample. In some embodiments, expression is expression in a liquid biopsy. In some embodiments, expression is percent expression. In some embodiments, percent is percent of cells. In some embodiments, the therapy is anti-PD-1 therapy and the protein in the process is PD-L1. In some embodiments, the therapy is anti-PD-L1 therapy, and the target protein is PD-L1. In some embodiments, the clinical parameter is PD-L1 expression. In some embodiments the training set comprises at least one clinical parameter selected from line of treatment, PD-L1 expression, sex and age. In some embodiments the training set comprises protein expression levels and sex. In some embodiments the training set comprises number of RAPs, age and PD-L1 status.

Additionally clinical parameters may also be included. A skilled artisan will be able to select relevant clinical parameters for inclusion in the training set. Examples of additional clinical parameters include, but are not limited to, histological type of the sample (e.g., adenocarcinoma, squamous cell carcinoma, etc.), metastatic location, tumor location, cancer staging (such as tumor, nodes and metastases, TNM, staging for example), performance status (such as ECOG performance status), genetic mutations, epigenetic status, general medical history, vital signs, blood measurements, renal and liver function, weight, height, pulse, blood pressure and smoking history.

In some embodiments, at an inference stage the trained machine learning algorithm is applied. In some embodiments, the trained machine learning algorithm is applied to individual received factor expression levels. In some embodiments, the trained machine learning algorithm is applied to individual received factor expression levels and the at least one clinical parameter. In some embodiments, the trained machine learning algorithm is applied to individual received factor expression levels from the subjects and the subject's sex. In some embodiments, the trained machine learning algorithm is applied to the number of resistance-associated proteins. In some embodiments, the trained machine learning algorithm is applied to the number of resistance-associated factors. In some embodiments, the trained machine learning algorithm is applied to the number of resistance-associated factors and at least one clinical parameter.

In some embodiments, at the inference stage an input is received. In some embodiments, the input comprises the number of resistance-associated factors expressed in a sample. In some embodiments, the sample is from a subject. In some embodiments, the input comprises at least one clinical parameter. In some embodiments, the subject suffers from the disease. In some embodiments, the subject has unknown responsiveness to the therapy. In some embodiments, the parameter is of the subject with unknown responsiveness. In some embodiments, at the inference stage the trained machine learning algorithm is applied. In some embodiments, applied is applied to the input. In some embodiments, the input is the received input. In some embodiments, the inference stage is to predict responsiveness. In some embodiments, responsiveness is responsiveness to the therapy of the subject with unknown responsiveness.

In some embodiments, the machine learning algorithm outputs the resistance score. In some embodiments, the outputted resistance score is scaled from 0 to 1. In some embodiments, 1 is perfectly similar to non-responders and 0 is perfectly similar to responders. In some embodiments, the machine learning algorithm calculates similarity to responders. In some embodiments, the machine learning algorithm calculates similarity to non-responders. In some embodiments, the machine learning algorithm outputs a numeric value of similarity to responders and non-responders. In some embodiments, a protein is considered to be a RAP if its resistance score is beyond a certain threshold. In some embodiments, the threshold for the resistance score is calculated on a scale of 0 to 1. In some embodiments, the threshold for the resistance score of a certain protein is between 0.2 and 0.95. In some embodiments, the threshold for the resistance score of a certain protein is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the resistance score is 0.25. In some embodiments, the threshold for the resistance score is 0.42. In some embodiments, the threshold for the resistance score is 0.6. In some embodiments, the threshold for the resistance score when calculated by a machine learning algorithm is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the resistance score when calculated with a machine learning algorithm is 0.25. In some embodiments, the threshold for the resistance score when calculated with a machine learning algorithm is 0.42. In some embodiments, the threshold for the resistance score when calculated with a machine learning algorithm is 0.6.

In some embodiments, response probability is determined by the calculation (1-resistance score). In some embodiments, 1-resistance score is 1-total resistance score. In some embodiments, the resistance score is the total resistance score. In some embodiments, response probability is a response score. In some embodiments, the machine learning algorithm outputs the response score. In some embodiments, the outputted response score is scaled from 0 to 1. In some embodiments, 1 is perfectly similar to responders and 0 is perfectly similar to non-responders. In some embodiments, the machine learning algorithm calculates similarity to responders. In some embodiments, the machine learning algorithm calculates similarity to non-responders. In some embodiments, the machine learning algorithm outputs a numeric value of similarity to responders and non-responders. In some embodiments, a protein is considered to be a RAP if its response score is beyond a certain threshold. In some embodiments, a protein is considered to be an active RAP if its response score is beyond a certain threshold. In some embodiments, the threshold for the response score is calculated on a scale of 0 to 1. In some embodiments, the threshold for the response score of a certain protein is between 0.2 and 0.95. In some embodiments, the threshold for the response score of a certain protein is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the response score is 0.25. In some embodiments, the threshold for the response score is 0.276. In some embodiments, the threshold for the response score is 0.42. In some embodiments, the threshold for the response score is 0.5. In some embodiments, the threshold for the response score is 0.6. In some embodiments, the threshold for the response score when calculated by a machine learning algorithm is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the response score when calculated with a machine learning algorithm is 0.25. In some embodiments, the threshold for the response score is 0.276. In some embodiments, the threshold for the response score when calculated with a machine learning algorithm is 0.42. In some embodiments, the threshold for the response score when calculated with a machine learning algorithm is 0.5. In some embodiments, the threshold for the response score when calculated with a machine learning algorithm is 0.6. In some embodiments, the algorithm outputs response probability, and the response probability is calculated on a scale of 0 to 1. In some embodiments, the algorithm outputs response probability, and the response probability is calculated on a scale of 0 to 10. In some embodiments, the algorithm outputs response probability, and the response probability is calculated on a scale of 0% to 100%, wherein 100% is a perfect responder and 0% is perfect non-responder. In some embodiments, a response probability above 50% indicates a subject likely to respond. In some embodiments, a response probability below 50% indicates a subject unlikely to respond. In some embodiments, the threshold for the response score when calculated with a machine learning algorithm is 0.25. In some embodiments, a protein with a response score above 0.25 is active in the subject. In some embodiments, the threshold for the response score when calculated with a machine learning algorithm is 0.5. In some embodiments, a protein with a response score above 0.5 is active in the subject. In some embodiments, the algorithm outputs clinical benefit probability. In some embodiments, the clinical benefit probability is calculated on a scale of 0 to 1. In some embodiments, a clinical benefit probability of 0 indicates a 0% likelihood of clinical benefit to the subject. In some embodiments, a clinical benefit probability of 1 indicates a 100% likelihood of clinical benefit to the subject. In some embodiments, the algorithm outputs clinical benefit probability, and the clinical benefit probability is calculated on a scale of 0 to 10. In some embodiments, a clinical benefit probability of 10 indicates a 100% likelihood of clinical benefit to the subject. In some embodiments, the algorithm outputs clinical benefit probability, and the clinical benefit probability is calculated on a scale of 0% to 100%. In some embodiments, a clinical benefit probability of 100% indicates a 100% likelihood of clinical benefit to the subject. In some embodiments, a clinical benefit probability of 0% indicates a 0% likelihood of clinical benefit to the subject. In some embodiments, greater than 50% likelihood of clinical benefit to the subject indicates the subject should continue or be administered the therapy. In some embodiments, the therapy is a monotherapy. In some embodiments, the therapy is a combination therapy. In some embodiments, the threshold for the clinical benefit probability is the median clinical benefit probability in the development set. In some embodiments, the threshold for the clinical benefit probability is the median clinical benefit probability in the development set, wherein a clinical benefit probability higher than the median clinical benefit probability is responder and a clinical benefit probability lower than the median clinical benefit probability is non-responder. According to some other embodiments, response probability or clinical benefit probability beyond 50% indicates the subject is responsive to therapy. According to some other embodiments, response probability or clinical benefit probability below 50% indicates the subject is non-responsive to therapy. In some embodiments, the response probability or the clinical benefit probability is from 0-10, and response probability or clinical benefit probability beyond 5 indicates the subject is responsive to therapy. In some embodiments, the response probability or the clinical benefit probability is from 0-10, and response probability or clinical benefit probability below 5 indicates the subject is non-responsive to therapy.

In some embodiments, the score is between zero and 1. In some embodiments, active is active in the cancer. In some embodiments, active is active in the subject. In some embodiments, active is active in promoting resistance. In some embodiments, beyond a threshold is below a threshold. In some embodiments, beyond a threshold is above a threshold. In some embodiments, the predetermined threshold is 0.5, 0.4, 0.3, 0.25, 0.2, 0.15, 0.1, 0.05, 0.01, 0.005, 0.001, 0.0005 or 0.0001. Each possibility represents a separate embodiment of the invention. In some embodiments, the threshold is 0.05. In some embodiments, the threshold is 5%. In some embodiments, the number of active RAPs is combined to give a total number of RAPs active in the subject. In some embodiments, the number of active RAPs is linearized to provide a total score between 0 and 1. In some embodiments, linearized is linearly scaled. In some embodiments, linearizing comprises a linear regression. In some embodiments, the number of active RAPs is converted to a total score between 0 and 1.

In some embodiments, the predetermined threshold is determined by performing a cross-validation within the training set. In some embodiments, the predetermined threshold is the median score in the training set. In some embodiments, the predetermined threshold is the score that best distinguishes between responders and non-responders in the training set.

In some embodiments, the machine learning algorithm outputs the resistance score. In some embodiments, the resistance score is the RAP score. In some embodiments, the outputted resistance score is scaled from 0 to 1. In some embodiments, 1 is perfectly similar to non-responders and 0 is perfectly similar to responders. In some embodiments, for a response score 1 is perfectly similar to responders and 0 is perfectly similar to non-responders. In some embodiments, the machine learning algorithm calculates similarity to responders. In some embodiments, the machine learning algorithm calculates similarity to non-responders. In some embodiments, the machine learning algorithm outputs a numeric value of similarity to responders and non-responders. In some embodiments, a protein is considered to be a RAP if its resistance score is beyond a certain threshold. In some embodiments, the threshold for the resistance score is calculated on a scale of 0 to 1. In some embodiments, the threshold for the resistance score of a certain protein is between 0.2 and 0.95. In some embodiments, the threshold for the resistance score of a certain protein is about 0.01, 0.05, 0.1, 0.15, 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the resistance score is 0.25. In some embodiments, the threshold for the resistance score is 0.42. In some embodiments, the threshold for the resistance score is 0.6. In some embodiments, the threshold for the resistance score when calculated by a machine learning algorithm is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the resistance score when calculated with a machine learning algorithm is 0.25. In some embodiments, the threshold for the resistance score when calculated with a machine learning algorithm is 0.42. In some embodiments, the threshold for the resistance score when calculated with a machine learning algorithm is 0.6.

In some embodiments, response probability is determined by the calculation (1-resistance score). In some embodiments, 1-resistance score is 1-total resistance score. In some embodiments, the resistance score is the total resistance score. In some embodiments, response probability is a response score. In some embodiments, the machine learning algorithm outputs the response score. In some embodiments, the outputted response score is scaled from 0 to 1. In some embodiments, 1 is perfectly similar to responders and 0 is perfectly similar to non-responders. In some embodiments, the machine learning algorithm calculates similarity to responders. In some embodiments, the machine learning algorithm calculates similarity to non-responders. In some embodiments, the machine learning algorithm outputs a numeric value of similarity to responders and non-responders. In some embodiments, a protein is considered to be a RAP if its response score is beyond a certain threshold. In some embodiments, beyond is above. In some embodiments, beyond is below. In some embodiments, the threshold for the response score is calculated on a scale of 0 to 1. In some embodiments, the threshold for the response score of a certain protein is between 0.2 and 0.95. In some embodiments, the threshold for the response score of a certain protein is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the response score is 0.25. In some embodiments, the threshold for the response score is 0.42. In some embodiments, the threshold for the response score is 0.6. In some embodiments, the threshold for the response score when calculated by a machine learning algorithm is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the response score when calculated with a machine learning algorithm is 0.25. In some embodiments, the threshold for the response score when calculated with a machine learning algorithm is 0.42. In some embodiments, the threshold for the response score when calculated with a machine learning algorithm is 0.6.

In some embodiments, the calculated resistance scores are combined to produce a total resistance score. In some embodiments, the calculated response scores are combined to produce a total response score. It will be understood by a skilled artisan that as the response and resistance scores are just 1 minus the other, they are always interchangeable. The conversion of resistance to response can be performed on the individual factor level or after the scores are combined and performed on the total level. In some embodiments, combine is sum. In some embodiments, the resistance scores are summed to produce a total resistance score. In some embodiments, combine is average. In some embodiments, the resistance scores are averaged to produce a total resistance score. In some embodiments, the scores are weighted when combined.

In some embodiments, the method comprises determining the number of factors of the plurality of factors that are active in the subject. In some embodiments, an active factor is a factor with a resistance score above a predetermined threshold. In some embodiments, the threshold is 0.25. In some embodiments, a factor with a resistance score above 0.25 is a factor active in the subject. In some embodiments, the threshold is 0.276. In some embodiments, a factor with a resistance score above 0.276 is a factor active in the subject. In some embodiments, only the active factors are combined. In some embodiments, the combining the calculated resistance scores is combining the active resistance scores. In some embodiments, combining comprises adding up the number of factors that are active in the subject. In some embodiments, the number of factors active in the subject is converted into a score from 0 to 1. In some embodiments, the number of factors active in the subject is converted into a score from 0 to 10. In some embodiments, converted comprises applying a linear regression model. In some embodiments, the number of active factors is linearized to provide a total score between 0 and 1. In some embodiments, the number of active factors is linearized to provide a total score between 0 and 10. In some embodiments, linearized is linearly scaled. In some embodiments, linearizing comprises a linear regression. In some embodiments, the threshold is 5.

In some embodiments, the machine learning model is a machine learning algorithm. In some embodiments, the algorithm is a supervised learning algorithm. In some embodiments, the algorithm is an unsupervised learning algorithm. In some embodiments, the algorithm is a reinforcement learning algorithm. In some embodiments, the machine learning model is a Convolutional Neural Network (CNN). In some embodiments, the at least one hardware processor trains a machine learning model. In some embodiments, the model is based, at least in part, on a training set. In some embodiments, the model is based on a training set. In some embodiments, the model is trained on a training set. In some embodiments, the at least one hardware processor applies the machine learning model to a factor expression level from a subject.

In some embodiments, the calculating comprises calculating a mean expression for each protein in responders. In some embodiments, the calculating comprises calculating a mean expression for each protein in non-responders. In some embodiments, the calculating comprises calculating a mean expression for each protein in responders and a mean expression for each protein in non-responders. In some embodiments, the calculating comprises calculating a distribution of the expression for each protein in responders and non-responders. In some embodiments, the calculating comprises calculating a standard deviation of expression for each protein in responders and non-responders. In some embodiments, in responders is in the responders population. In some embodiments, in non-responders is in the non-responders population. In some embodiments, the resistance score is based on the ratio of deviation of the factor expression in the subject from the calculated mean in responders to the deviation of the factor expression in the subject from the calculated mean in non-responders. Calculation of deviation is well known to one skilled in the art. It will be understood that the more dissimilar the expression in the subject is from a mean the larger the deviation will be. Thus, factors that are very dissimilar to the mean in responders will have a large numerator in the calculation of this ratio and factors that are lowly dissimilar to the mean in non-responders will have a small denominator. Thus, the more dissimilar to responder expression and the more similar to non-responder expression is expression of a factor in a subject the higher the resistance score will be. In some embodiments, a resistance score beyond a predetermined threshold indicates a factor is a resistance-associated factor. In some embodiments, a resistance-associated factor is a resistance-associated protein (RAP). In some embodiments, resistance-associated factor is a RAP if its expression in responders is statistically different from its expression in non-responders.

In some embodiments, the calculating further comprises calculating a distribution for each factor in responders. In some embodiments, the calculating further comprises calculating a distribution for each factor in non-responders. In some embodiments, the calculating further comprises calculating a distribution for each factor in responders and a distribution for each factor in non-responders. In some embodiments, the calculating further comprises calculating a standard deviation for each factor in responders. In some embodiments, the calculating further comprises calculating a standard deviation for each factor in non-responders. In some embodiments, the calculating further comprises calculating a standard deviation for each factor in responders and a standard deviation for each protein in non-responders. In some embodiments, the calculating further comprises calculating a standard deviation for each factor in a mix of responders and non-responders. In some embodiments, the deviation is measured as a multiple of the calculated standard deviation. It will be understood by a skilled artisan that by scaling the deviation to the standard deviation for a group of expression values the deviation can be given in more absolute terms allow for the comparison of factors and populations with very small and very large stand deviations (which may also have very low and very high expression levels).

In some embodiments, the resistance score is based on a Z-score for the expression level of each factor in the subject. In some embodiments, the resistance score is based on the Z-score relative to responders. In some embodiments, the resistance score is based on the Z-score relative to non-responders. In some embodiments, the resistance score is based on both the Z-score relative to responders and the Z-score relative to non-responders. In some embodiments, the resistance score is based on the ratio of the Z-score relative to responders to the Z-score relative to non-responders. It will be well known to a skilled artisan that a Z-score counts the distance of the individual level from the population mean in units of the population standard deviation. In some embodiments, the Z-score is calculated by Equation 1.

In some embodiments, the resistance score is calculated by the equation

$$( \frac{\vert {Z}_{R} \vert}{\vert {z}_{NR} \vert + c} ) .$$

In some embodiments, Z_{R} is the deviation of the factor expression in the subject from the calculated mean in responders. In some embodiments, Z_{NR} is the deviation of the factor expression in the subject from the calculated mean in non-responders. In some embodiments, | | is the Z-score of the deviation. In some embodiments, | | is the standardizing of the deviation to a multiple of the standard deviation. In some embodiments, c is a constant. In some embodiments, constant is a regulation constant that prevents the score from divergence for Z_{NR}=0. In some embodiments, the resistance score is calculated by Equation 2. In some embodiments, monotonoic is an ad-hoc function that prevents the resistance score from decreasing for extreme values within the non-responder distributions. In some embodiments, function is the function provided in Algorithm 1.

In some embodiments, a resistance score beyond a predetermined threshold indicates a factor is a RAP. In some embodiments, beyond is above. In some embodiments, the threshold is a predetermined threshold. In some embodiments, threshold is a threshold value. In some embodiments, the threshold for the resistance score is about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. Each possibility represents a separate embodiment of the invention. In some embodiments, the threshold is about 0.05, 0.1, 0.15, 0.2, 0.25, 0.3, 0.35, 0.4, 0.45, 0.5, 0.55, 0.6, 0.67, 0.7, 0.75, 0.8, 0.85 or 0.9. Each possibility represents a separate embodiment of the invention. In some embodiments, the threshold for the resistance score is about 2.9. In some embodiments, the threshold for the resistance score is 2.9. In some embodiments, the threshold for the resistance score is about 3.0. In some embodiments, the threshold for the resistance score is 3.0. In some embodiments, the threshold for the resistance score is calculated on a scale of arbitrary units. In some embodiments, the threshold for the resistance score when calculated by a mathematical calculation is about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, or 5.0. Each possibility represents a separate embodiment of the invention. In some embodiments, the threshold for the resistance score when calculated with a mathematical calculation is about 2.9. In some embodiments, the threshold for the resistance score when calculated with a mathematical calculation is 2.9. In some embodiments, the threshold for the resistance score when calculated with a mathematical calculation is about 3.0. In some embodiments, the threshold for the resistance score when calculated with a mathematical calculation is 3.0. In some embodiments, a mathematical calculation is a method that comprises calculating a mean expression for each protein.

In some embodiments, a subject with a number of resistance-associated factors (e.g., RAPs) above a predetermined number is predicted to be resistant to the therapy. In some embodiments, a subject with a number of resistance-associated factors above a predetermined number is predicted to not respond to the therapy. In some embodiments, a subject with a number of resistance-associated factors above a predetermined number is predicted to be a non-responder to the therapy. In some embodiments, a subject with a number of resistance-associated factors below a predetermined number is predicted to be suitable to the therapy. In some embodiments, a subject with a number of resistance-associated factors below a predetermined number is predicted to respond to the therapy. In some embodiments, a subject with a number of resistance-associated factors below a predetermined number is predicted to be a responder to the therapy. In some embodiments, a subject with a number of resistance-associated factors at or below a predetermined number is predicted to be suitable to the therapy. In some embodiments, a subject with a number of resistance-associated factors at or below a predetermined number is predicted to respond to the therapy. In some embodiments, a subject with a number of resistance-associated factors at or below a predetermined number is predicted to be a responder to the therapy.

In some embodiments, the predetermined number is a threshold number. In some embodiments, the predetermined number is 2, 3, 4, 5, 6, 7, 8, 9, 10, 11, 12, 13, 14, 15, 16, 17, 18, 19 or 20. Each possibility represents a separate embodiment of the invention. In some embodiments, the predetermined number is 3. In some embodiments, the predetermined number is 4. In some embodiments, the predetermined number is 7. In some embodiments, the predetermined number is 13.

In some embodiments, the method further comprises classifications of the resistance-associated factors into at least one pathway, process, or network. In some embodiments, the method further comprises performing analysis on resistance associated factors to determine at least one pathway, process, or network in which the resistance-associated factors are involved. In some embodiments, the pathway, process, or network causes non-responsiveness to the therapy. In some embodiments, the analysis is selected from pathway analysis, process analysis and network analysis. In some embodiments, the method further comprises performing pathway analysis on RAPs. In some embodiments, the method further comprises performing process analysis on RAPs. In some embodiments, the method further comprises performing network analysis on RAPs. In some embodiments, at least one pathway, process or network comprises at least 2, 3, 4, 5, 6, 7, 8, 9 or 10 pathways, processes, or networks. Each possibility represents a separate embodiment of the invention. In some embodiments, at least one pathway, process or network is all the pathways, processes or networks known to include the resistance associated factors. In some embodiments, at least one pathway, process or network is all the pathways, processes or networks enriched with resistance associated factors. In some embodiments, enriched is the most enriched. In some embodiments, enriched comprises contains the most RAPs of any or the pathways, processes or networks.

In some embodiments, the method comprises selecting a pathway, process or network. In some embodiments, the selected pathway, process or network is hypothesized to affect non-response to the therapy. In some embodiments, the selected pathway, process or network is hypothesized to cause non-response to the therapy. In some embodiments, the selected pathway, process or network is known to be druggable. In some embodiments, known to be druggable comprises a known therapeutic agent that modulates the pathway, process or network. In some embodiments, the known therapeutic agent is in or has concluded clinical trials. In some embodiments, the known therapeutic agent is approved for human use. In some embodiments, approved for human use is approved for use in treating the disease in a human. In some embodiments, the disease is cancer. In some embodiments, the method further comprises administering to a subject that is a non-responder, or predicted to be a non-responder, an agent that modulates the at least one pathway, process, or network containing a resistance associated factor. In some embodiments, the agent inhibits a target in said pathway, process, or network. In some embodiments, the target is a gene. In some embodiments, the target is a protein. In some embodiments, the protein is a regulatory RNA. In some embodiments, the target is a response associated factor. In some embodiments, the target is not a response associated factor. In some embodiments, the agent activates a target in the pathway, process, or network. In some embodiments, the agent modulates the pathway, process or network. In some embodiments, the pathway's activity induces non-response, and the agent inhibits the pathway. In some embodiments, the pathway's activity reduces non-response, and the agent activates the pathway. It will be understood by a skilled artisan that a response associated factor is identified by its expression in a subject being more similar to the expression in non-responders than responders. Thus, for example, if the factor is more highly expressed in non-responders and increases activity of the pathway/process/network then the agent would inhibit the pathway. If, for example, the factor is more highly expressed in non-responders, but decreases activity of the pathway/process/network then the agent would activate the pathway/process/network. Similarly, if the factor, for example, is more lowly expressed in non-responders and decreases activity of the pathway/process/network the agent would inhibit the pathway/process/network. And lastly, if, for example, the factor is more lowly expressed in non-responders but increases activity of the pathway/process/network the agent would activate the pathway/process/network. Essentially, the agent should induce the pathway/process/network to function more as it does in responders. In some embodiments, the agent targets a hub target in the pathway. In some embodiments, the agent targets a regulator target in the pathway. In some embodiments, the process activity induces non-response, and the agent inhibits the process. In some embodiments, the processes' activity reduces non-response, and the agent activates the process. In some embodiments, the agent targets a hub target in the process. In some embodiments, the agent targets a regulator target in the process. In some embodiments, the network activity induces non-response, and the agent inhibits the network. In some embodiments, the network activity reduces non-response, and the agent activates the network. In some embodiments, the agent targets a hub factor in the network. In some embodiments, the agent targets a regulator factor in the network. In some embodiments, the regulator is a master regulator. The factors can be classified into pathways, protein interaction or signals using any analysis tool known in the art. Examples include, but are not limited to, GO analysis, Ingenuity analysis, Metacore analysis (Clarivate Analytics), reactome pathway analysis and functional analysis.

By another aspect there is provided, a computer program product comprising a non-transitory computer-readable storage medium having program code embodied thereon, the program code executable by at least one hardware processor to perform a method of the invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Patient cohort and specimen collection: Blood plasma samples and clinical data were collected from 610 advanced stage NSCLC patients receiving ICI-based treatment at 20 participating medical centers. Comprehensive clinical data were collected for each patient and validated by comparing with source documentation. All patients were treated with ICI-based regimens including single agent ICI (pembrolizumab, atezolizumab or nivolumab), a combination of ICI and chemotherapy (pembrolizumab/atezolizumab plus chemotherapy) or an ICI combination (ipilimumab plus nivolumab). Inclusion criteria were: provision of informed consent; age older than 18 years; stage IIIB-IV NSCLC; ECOG performance status 0-2; normal hematological, renal and liver functions. In addition, exclusion criterion was any concurrent and/or other active malignancy that required systemic treatment within 2 years prior to receiving the first dose of ICI-based treatment. The overall cohort size was set when the performance was stable in the development set.

Specimens were collected prior to commencement of treatment, either immediately before the first treatment dose on the same day (n=244), or within 10 days (n=52), 11-30 days (n=38) or 31-58 days (n=5) prior to starting treatment (the numbers refer to the cohort after patient exclusion; please see patient exclusion section for additional details). Specimen collection was performed as follows: blood samples were collected from each patient into EDTA-anticoagulated tubes; plasma was isolated from whole blood by centrifugation at 1200×g at room temperature for 10-20 minutes within 4 hours of venipuncture; plasma supernatant was collected and stored frozen at −80° C. and were shipped frozen to the analysis laboratory.

A separate retrospective cohort comprised of 85 patients receiving chemotherapy was included for certain comparisons. In addition to the ICI-based cohort, a retrospective cohort of patients receiving chemotherapy as a monotherapy was assembled. The samples were collected using the same protocol between September 2015 and October 2018. Inclusion criteria: advanced stage NSCLC undergoing first-line chemotherapy treatment without changing to ICI treatment or adding ICIs to the treatment regimen. For comparison between ICI-based therapy and chemotherapy cohorts, patient baseline characteristics were compared between the ICI-based development set and the chemotherapy set using Chi-square test for categorical data and t-test for continuous variables.

Assessment of therapeutic benefit: Clinical benefit data were retrieved from patient medical records and verified by the investigators through a review of radiologic images, i.e., CT chest/abdomen and brain MRI performed every 2-3 months, based on Response Evaluation Criteria In Solid Tumors (RECIST) 1.1. Clinical benefit (CB) was also assessed based on Progression Free Survival (PFS) at 12 months after the commencement of treatment. Therapeutic benefit was assessed based on progression event at 12 months. Since a patient may arrive to the 12 month-clinical evaluation some time before or after 12 months, we decided to examine the range of 330 and 400 days after commencement of treatment as following; patients were assigned as having clinical benefit (CB) if progression event was determined beyond 400 days, or if until 330 days there was no progression; Patients were assigned as having no clinical benefit (NCB) if there was a progression until 400 days following treatment initiation (including); in case there was no progression event until day 330 (including), the patient was regarded as ‘no clinical benefit label’ and was excluded from the classifier development or validation process.

Alternatively, therapeutic benefit was assessed at 3, 6 and 12 months after commencement of treatment, and patients were assigned clinical benefit (CB) or no clinical benefit (NCB) classifications per time point. At 3 and 6 months, patients displaying complete response, partial response or stable disease were classified as CB patients, whereas patients displaying progressive disease or who had died were classified as NCB patients. Durable clinical benefit was assessed 12 months after commencement of treatment. Patients who were alive with confirmed absence of progressive disease for at least 12 months after starting treatment were classified as CB patients. Patients who stopped treatment before the 12-month mark due to treatment-related adverse events (but displayed no signs of progression for at least 12 months) were also classified as CB patients. All other patients were classified as NCB patients. All patients were followed up for at least 2 years. The time at which progressive disease and/or death occurred was recorded. If there was a change in treatment due to treatment-related adverse events or patient refusal to continue treatment, only those patients who received 2 or more ICI cycles remained in the study. Patients who stopped chemotherapy, but continued ICI therapy remained in the study.

Proteomic measurements, data normalization and quality control: Proteomic profiling of plasma samples was performed using an assay that simultaneously measures approximately 7000 protein targets. The assay is based on chemically-modified single stranded oligonucleotides that fold into molecular structures capable of binding to proteins with high affinity and specificity. The measurement is performed using DNA microarray technology with a readout provided in relative fluorescence units (RFU). The assay simultaneously measures a total of 7596 protein targets, out of which 7289 targets are human proteins.

Cohort samples were run in two running batches. Each sample was profiled once. Quality control and standardization were performed. Since protein level distributions are roughly log-normal (i.e., the logarithm of the measurement is normally distributed), and given that many statistical methods assume normality, log 2 transformation was applied unless stated otherwise. There were no data imputations in the model development and validation. When a patient had a ‘not available’ (NA) data entry in a clinical parameter, the entry was treated as NA.

The proteomic dataset was narrowed down to a set of proteins with high analytical reliability by comparing the proteomic dataset of the current cohort to that of a distinct cohort not participating in the study. For each assayed protein, the expression level distributions were compared between the two cohorts by applying the Kolmogorov-Smirnov test. Proteins with a p-value below 0.05 were excluded, resulting in 1578 proteins for model development.

Model development and evaluation was performed on patients receiving ICI-based therapy who had clinical benefit evaluation. The model was constructed on the development set (n=228) and tested in a blinded manner on the independent validation set (n=272).

### Patient Exclusion:

Of the 610 enrolled patients, 65 were excluded due to technical or clinical reasons (eFigure1 in supplemental). Ten patients did not pass SomaScan® quality check or had missing measurements. Samples from 13 patients were excluded as they were obtained not within the time frame defined for blood collection (blood was collected more than 2 months before treatment or after ICI-based treatment). Thirty-two patients were excluded due to treatment-related issues (did not receive therapy; not naïve to immunotherapy; received chemotherapy less than 60 days prior to ICI treatment; received ipilimumab combined with nivolumab; notably, the latter group was excluded since this is a different treatment compared to anti PD-L1 with or without chemotherapy, and 16 patients in this category are not enough for a robust analysis. Future research will include these patients when the group size will be sufficiently large). Ten patients were excluded due to eligibility issues (ECOG above 2; mental health disorder; driver mutations; multiple cancer types). Following patient exclusion, 545 patients remained in the dataset.

For each downstream analysis a different exclusion of the ICI-based cohort was performed; For the development and validation of the PROphet model, the analysis required patients with clinical benefit evaluation and only first-line ICI treatment in the validation set, leaving 500 patients for this analysis; For the analysis that involved the combination of PROphet with PD-L1 expression level, patients without PD-L1 evaluation were excluded, as well as patients with advanced/unknown line of treatment, leaving 444 ICI-based therapy patients in the analysis.

Resistance Associated Protein (RAP) model development and validation: To avoid data leakage, the cohort was divided into development and validation sets. The model was constructed on the development set (n=228). After construction of the final model, a blinded validation was performed on the validation set (n=272). After the model development was completed and the model configuration was locked, proteomic and clinical data was acquired for additional 272 patients, that constitutes the validation set, and a blinded validation was performed on this set of samples. Notably, the data of the validation set data were not available at the time the model was develop; while this practice guarantees that the validation is truly blinded, it was not possible to assure that the distributions of various clinical parameters are similar between the development and the validation sets. Indeed, few clinical parameters displayed a statistically significant difference between the development and validation sets (sex, ECOG, PD-L1 expression levels and age). It is important to emphasize that while similar distributions of clinical parameters are desired, it is impossible to achieve when the validation set is constructed after the model development is completed. Moreover, the model is expected to perform best when applied to similar populations; therefore, differences between the development and validation sets exert additional stress on the model. The same division into development (n=228) and validation (n=272) sets described above was applied for the PD-L1 based prediction model and prediction model. To improve performance, the PD-L1 model was based on numeric values of PD-L1 rather than categorical values (i.e., PD-L1≥50%, PD-L1 between 1% and 49% and PD-L1<1%); since not all samples had numeric values, 210 and 204 patients were in the development and validation sets of this model, respectively.

The model was developed using a random sampling approach with multiple iterations. In each iteration the development set was randomly divided into a train set and a test set (75% and 25% of the development set, respectively). In each iteration, the training set was used for feature selection and model training in the following manner: Proteins displaying differential levels between CB and NCB patients were identified using Kolmogorov-Smirnov test. A prediction model based on a single protein was constructed on the iteration train set for each of the 50 proteins with the lowest p-value (i.e., 50 independent models were constructed, where each model is based on a single protein). XGBoost algorithm was used for the construction of each single protein model using two features, namely the protein expression level and the patient's sex. Sex was included in the model as a feature since it affects the plasma expression level of the protein (this way the model was not biased toward the majority of patients, which are males). The output of each single protein model is a probability between 0 and 1—where the lower the probability is, the more likely is the patient to display clinical benefit. The overall patient score at each iteration was extracted by summing the number of single protein model that are indicative of NCB, where a single-protein model was indicative of NCB if its predicted probability was above the observed cohort CB rate (=0.276). Following this methodology, the output of the iteration model is an integer between 0 and 50, where a number close to 0 corresponds to CB and a number close to 50 corresponds to NCB. The steps described above for a single iteration were repeated for 80 iterations, where the overall patient outcome is its average outcome of the 80 iterations. Finally, the model score was linearly scaled to values between 0 and 10, where values below 5 indicate a NEGATIVE result, while values equal or greater than 5 indicate a POSITIVE result.

Model performance was evaluated on the independent validation set in a blinded manner using two metrics: (i) Agreement between the predicted CB probability and the observed CB rate in terms of goodness of fit (R2 of a linear regression), where the observed CB rate for each CB value was defined as the proportion of CB patients among a group of patients within the range of the CB probability ±0.05 window. (ii) By examining the hazard ratio (HR) for the positive population vs. the negative population, as calculated using Cox proportional hazard model. Additional prediction models: To maintain consistency with the RAP model, all prediction models described in this study underwent a similar development pipeline. First, the same development and validation sets used for the RAP model were used for the other models. Second, the development set was randomly divided into train and test sets (75% and 25% of the development set, respectively) 80 times. In each iteration, the model was developed on the train set using the XGBoost algorithm and predictions were inferred on the test set. The predictions from all iterations were averaged and returned as CB probabilities. For the PD-L1-based model, PD-L1 status was the only input (high, low or negative). For the clinical model, four clinical parameters were used as input: (i) PD-L1 status (high, low or negative); (ii) ECOG performance status; (iii) patient sex; (iv) line of treatment (first or advanced). Integrated models (i.e., RAP model combined with another model) were developed in two steps. In the first step, the RAP model was developed as described above. In the second step, the output of the RAP model served as an input feature along with the relevant clinical parameters. The development set was again divided into train and test sets 80 times, each time with a new division into train and test sets, and predictions from all iterations were averaged. Model output was CB probability. Performance assessment and comparison was performed using ROC curves and linear regression between predicted CB probability and observed CB rate, as described above.

Data analysis: All data analyses were conducted using Python, Perseus computational platform and GraphPad Prism (San Diego, California, USA, graphpad.com). Multivariate Cox proportional hazard regression with the stepwise model reduction procedure was used to obtain hazard ratios for treatment effect, adjusted to all other factors, and to assess the interaction between treatment and prediction class. Factors that were initially found to have an effect on the hazard ratio, were also tested for interaction with treatment. Hazard ratios are reported with 95% confidence intervals and p-values. A level of 0.05 or lower was considered significant. The R statistical software was used for analysis by the packages Survival and MASS. For the overall survival and the progression-free survival analyses, the 444 patients first-line ICI-treated patients with determined PD-L1 levels, along with the chemotherapy cohort (n=85), were examined.

Associations between CB and clinical parameters were evaluated using χ₂ test for categorical parameters or t-test for numerical parameters. The network of RAPs was generated based on STRING database. Voronoi plots for the proteins in each consensus cluster were plotted using Proteomaps. Enrichment analysis for the CB probability values was done using 2D enrichment test (false discovery rate <0.05) [37]. Enrichment analysis for the RAPs selected in at least 10 iterations was done using Fisher exact test against the overall background of 1578 examined proteins (false discovery rate <0.1). Enrichment analyses for RAP functionality was performed using different iteration number cutoffs and resulted in similar results. The protein categories were based on Human Protein Atlas (proteinatlas.org), CHAT, ECM maristome project, and UniProt (keywords).

Statistical analysis: Log-rank and multivariate Cox proportional hazard regression tests were used to obtain hazard ratios for treatment effect while accounting for prediction class and adjusting for effects of other patient covariates.

### Example 1: Response Prediction Based on Resistance Associated Proteins (RAPs)-Proof of Concept

### Data Collection

The response prediction proof of concept was based on analysis of blood samples from 108 Non-Small Cell Lung Cancer (NSCLC) patients under Immune Check Inhibitor (ICI) treatment. The various administered treatments are summarized in Table 1.

TABLE 1

| Treatment | Number of Patients |
|---|---|
| Pembrolizumab, Chemotherapy | 41 |
| Pembrolizumab | 37 |
| Nivolumab | 12 |
| Ipilimumab, Nivolumab | 6 |
| Treatment unknown | 3 |
| Ipilimumab, Nivolumab, Chemotherapy | 4 |
| Atezolizumab | 3 |
| Nivolumab, Chemotherapy | 2 |

Plasma protein levels in the 108 patients were measured, in which approximately 1100 non-redundant protein targets are measured. Samples were taken before initiation of ICI treatment (T0) and after the first treatment was administered (T1) for a total of 156 samples in the batch.

### Classifier Construction

To predict response to treatment, the proteomic levels and the response labels were incorporated by a supervised learning algorithm. The response labels were responders (R) and non-responders (NR) and were determined based on the Overall Response Rate (ORR) assessment at 3 months. Specifically, progressive disease (PD) or early death associated with disease progression was classified as NR. Stable Disease (SD), Minimal Response (MR), Partial Response (PR) and Complete Response (CR) were classified as R. The ORR assessment was performed as described in clinical trial NCT04056247 (clinicaltrials.gov/ct2/show/NCT04056247, herein incorporated by reference in its entirety) in the “Primary Outcome Measures” section, by RECIST 1.1 or other validated method for ORR evaluation. Changes in the blood levels of different proteins that represent the host response [Time Frame: At baseline (pre-therapy, T0) and after 1^{st} treatment administration (post therapy), T1] were determined as described.

The samples were divided into a training set and a test set. All the development stages of the algorithm were performed using the training set while the test set was used only at the final stage to test the performance of the final algorithm. The training set included samples from n=78 patients (59 responders and 19 non-responders), and the test set included the samples analyzed in n=30 patients.

The response classifier treats features as an input and predicts response based on feature values. The features are the protein levels measured in the plasma at the two time points—at baseline (T0) and following the first treatment (T1). Measurements of the same protein at different time points are regarded as independent features. Moreover, some proteins have more than one measurement in a single proteomic profile (for example, the protein IL-6 is measured four times). Each repeat was treated as an independent feature.

### Resistance Associated Proteins

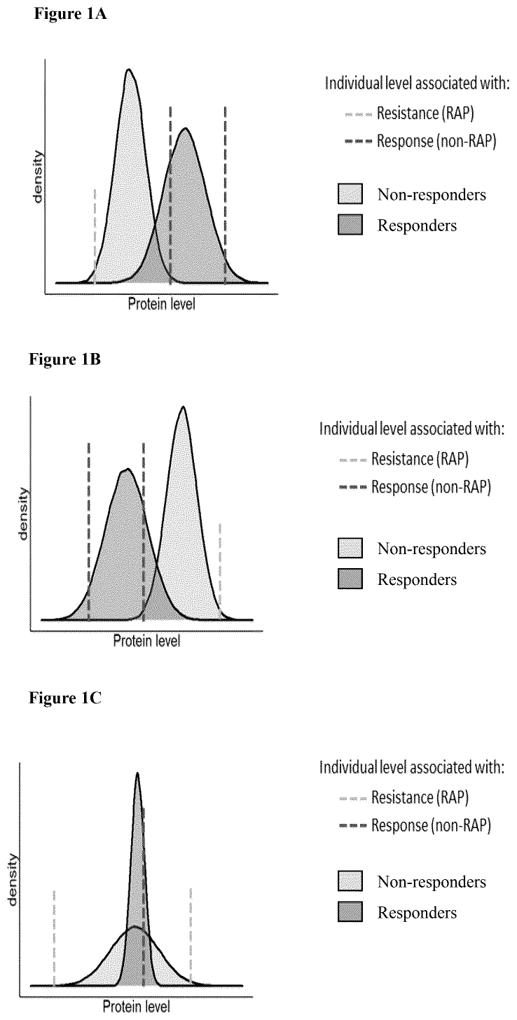

A resistance associated protein (RAP) refers to a specific protein whose expression in a given patient confers resistance to therapy, i.e., RAPs are patient specific. A protein is considered to be a RAP when its expression level in the respective patient is more similar to its expression distribution in the non-responder population than to the responder population (see FIGS. 1A-1C for illustrations). RAPs can be determined in a variety of ways. Provided herein is a mathematical calculation of RAPs as well as a machine learning algorithm for classifying RAPs and a method that combines the two. These methods are merely exemplary and any method of calculating RAPs may be employed.

To put the above concept into quantitative terms, a RAP score (i.e., a resistance score) was determined for each protein. A low RAP score value represents an expression level which is typical to the responder population, and a high RAP score indicates an expression level which is typical to the non-responder population. A protein is considered a RAP in cases where its RAP score is beyond (e.g., above or below depending on the construction of the score) a certain threshold. The RAP score threshold optimization process is described hereinbelow.

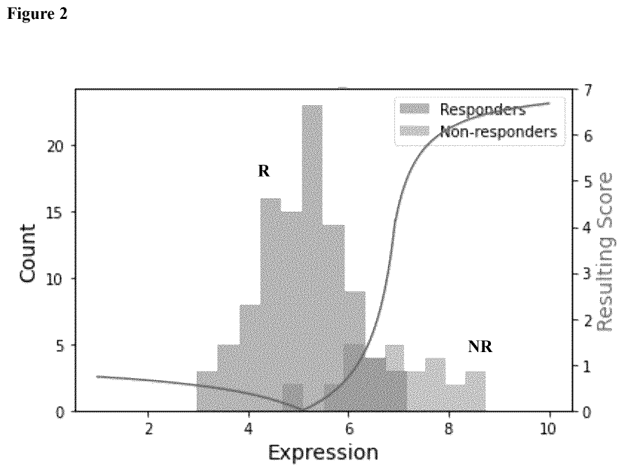

The RAP score calculation requires knowing the expression level distribution of each protein in both responder and non-responder populations, and data on the protein level expression of the tested patient. To allow comparison between several different proteins at different ranges of expression level, it is important that the RAP score will not be affected by and sensitive to the protein level expression scale. This is especially important in plasma samples, where there is a large dynamic range of 11 orders of magnitude in protein expression levels. To achieve this, the RAP score is based on Z-score, which counts the distance of the individual level from the population mean in units of the population standard deviation. In technical terms, Z-score is defined by Equation 1.

$$Z = \frac{x - μ}{σ} \qquad Equation 1$$

where x is the protein level in the tested patient, μ is the mean protein level in the population, and σ is the population standard deviation. The Z-score of a given patient is calculated separately with respect to the responders and non-responders populations. For the calculation of the Z-score relative to the responder population, noted by Z_{R}, the distribution measures, μ and σ, are calculated by using the responder population. For the calculation of the Z-score relative to the non-responder population, noted by Z_{NR}, the distribution measures, μ and σ, are calculated by using the non-responder population. Finally, the RAP score is defined by 2,

$$monotonic ( \frac{\vert {Z}_{R} \vert}{\vert {z}_{NR} \vert + c} ) \qquad Equation 2$$

where c is a regularization constant that prevents the score from divergence for Z_{NR}=0, and monotonoic is an ad-hoc function that was designed to prevent the RAP-score from decreasing for extreme values within the non-responder distributions. The function implementation is given by pseudo-code in Algorithm 1. RAP score values for representative responder and non-responder distributions are shown in FIG. 2.

Algorithm 1: The monotonic function used in Equation 2.

```
if |mean(R) − mean(NR)| > c · std(NR) then
  if mean(NR) > mean(R) then
      $sign ( mean ( NR ) - x ) \cdot RAP Score + \frac{( x > mean ( NR ) \cdot 2 \cdot \vert {Zscore}_{R} \vert )}{c}$
  else
      $sign ( x - mean ( NR ) ) \cdot RAP Score + \frac{( x < mean ( NR ) \cdot 2 \cdot \vert {Zscore}_{R} \vert )}{c} .$
```

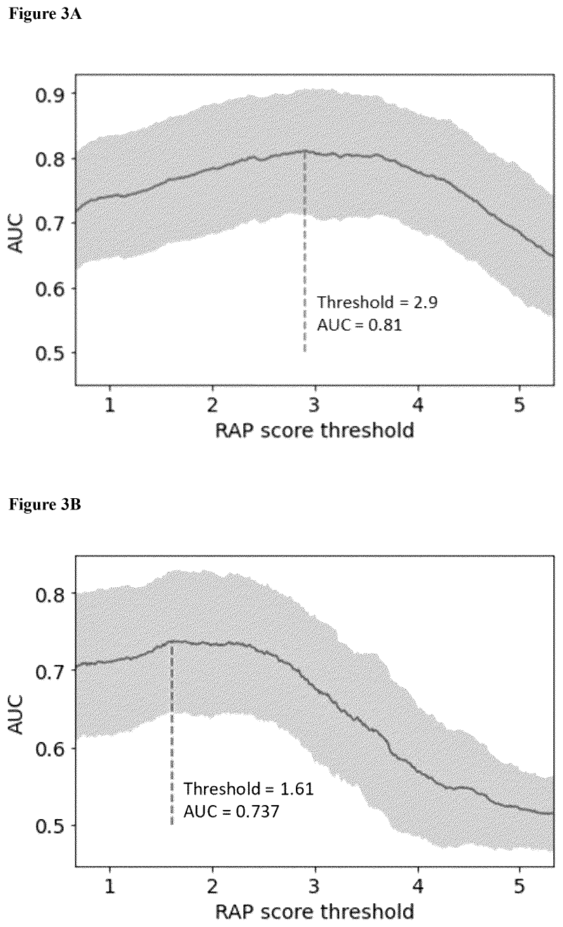

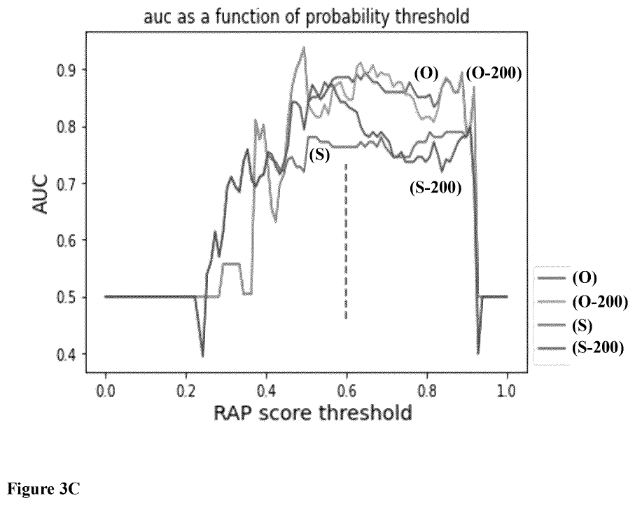

To determine the exact number of RAPs for a given patient, a threshold was determined for all proteins, wherein a protein with a RAP score above the determined threshold was considered as a RAP. The threshold was determined using cross-validation which is applied on the training set. Specifically, a cross-validation data set consisting of one third of the training set and a non-cross validation data set consisting of an additional one-third of the training set were sampled, while keeping the number of responders and non-responders similar between cross-validation and non-cross validation data sets. The calculation was performed on the non-cross-validation set and then for each patient in the cross-validation data set, a RAP score was calculated for every feature (i.e., all measured proteins at T0 and T1) using the responder and non-responder expression level distributions. The number of RAPs was then used to predict the response and receiver operating characteristics (ROC) area under the curve (AUC) quantifying the prediction performance was calculated for each threshold value (FIG. 3A-3B). To minimize the noise associated with a small dataset, 100 realizations were performed for each threshold value (i.e., different sampling of the cross-validation set from the training set) and the average AUC across the 100 realizations was considered. Notably, the mean ROC AUC curve of FIG. 3A demonstrates a single wide peak, suggesting that the prediction power of the number of RAPs is not very sensitive to the selected threshold. For features that include measurements at T0 and T1, the threshold was set to 1.61 (FIG. 3B) and 2.9 (FIG. 3A), respectively.

Machine learning evaluation: Although a purely mathematical approach is powerful (both conceptually and practically), it has several disadvantages that should be addressed:
1. The RAP score function depends on the underlying distribution of the protein expression level, hence its effectiveness may be platform dependent (in particular, as different proteomic systems use different measurement, units that do not scale naturally).
2. The current implementation does not provide a natural way to include clinical parameters (such as patient condition, indication details, treatment details, etc.) in the predictor.

An alternative approach making use of decision tree learning based on a machine learning algorithm to classify proteins as RAPs for a given subject was invented. For each measured protein a prediction model was generated using a machine learning algorithm (e.g., XGBoost algorithm) and based on the data of the training set. Such data from the training set may include not only protein expression levels and responder/non-responder tags, but also other features such as patient age, sex, condition, type of treatment, line of treatment, biomarkers expression such as PD-L1 expression etc. This approach makes no assumptions on the protein distribution and offers a natural framework to utilize clinical parameters.

To test this approach, samples from a cohort of 76 patients were screened using two different protein analysis platforms: approximately 1200 proteins (O) and the other measuring approximately 7500 proteins(S), with about 1000 proteins being common to both platforms. The treatment administered to these subjects is summarized in Table 2.

TABLE 2

| Treatment | Number of patients |
|---|---|
| Pembrolizumab | 28 |
| Pembrolizumab, Chemotherapy | 27 |
| Nivolumab | 5 |
| NA | 3 |
| Ipilimumab, Nivolumab | 4 |
| Ipilimumab, Nivolumab, Chemotherapy | 4 |
| Atezolizumab | 3 |
| Nivolumab, Chemotherapy | 2 |

The cohort of the 76 patients was divided into a training set that included 51 subjects (38 responders and 13 non-responders) and a test set that included 25 subjects (19 responders and 6 non-responders). The XGBoost algorithm was selected for this analysis due to the non-linear nature of the problem and the algorithms reputation of efficiency with learning on small data sets. In order to avoid multiple comparisons on the test set that will increase the risk of false discovery, and as the study goal is to verify the prediction feasibility (rather than identifying the optimal model configuration), the following predetermined configuration was used for the training model:

Model hyperparameters were set to:
a. Max tree depth=4
b. Ridging factors: eta=0.8, lambda=5, alpha=2
c. num_parallel_tree=100
d. objective=binary: logistic
e. eval_metric=logloss
   The parameters were selected in order to handle a small, noisy data set.

For the purposes of this evaluation the machine learning algorithm was trained only on protein expression levels while other considerations were excluded. Patient expression results were evaluated for each protein separately, and protein classifier was calculated for each single protein. The machine learning algorithm outputs a score from 0 to 1, with 1 being most similar to non-responders and 0 being most similar to responders.

Two configurations of input proteins were used to evaluate this approach. In the first configuration, all proteins were used as potential predictors. This is similar to what was employed in the mathematical approach, however, while for large cohorts this method is expected to be effective, for a small cohort size (compared to the number of features) false detection may hinder the predictive capability. In the second configuration, ranking the single protein models according to their tendency to partition the patients to responders and non-responders (i.e., give a higher rank to a protein model that has more balanced prediction classes) was used. As an extreme example, if a model predicted that all patients belong to a single class (responders or non-responders) the model received the lowest possible balance rank. On the other side of the scale, a model that divided the population evenly between responders and non-responders received the highest balance rank. After ranking the different protein models, the machine learning approach was evaluated using the 200 proteins with the highest balance rank.

Both approaches were used to evaluate the subjects based on their “O” and “S” expression values at T0 and T1. The model performance for “O” (measured by AUC) was above 0.8 in the threshold range of 0.4-0.8, with a stable and smooth behavior (FIG. 3C), peaking at an AUC=0.89 and 95% confidence interval of [0.594, 0.995]. Thus, for these samples the threshold was set at about 0.6. This result is a minor improvement to the AUC=0.846 obtained for the same data set using the mathematical RAP approach. However, due to the large confidence intervals (that are a result of the small data set size) the statistical significance of this difference is moderate.

The peak model performance for “O” when restricting the predictor to the 200 proteins was AUC=0.91 and 95% confidence interval of [0.602, 0.996] (FIG. 3C). The threshold was essentially the same in this case and the AUC represents a slight improvement to the full protein set configuration.

The model performance for “S” (measured by AUC) was above 0.75 in the threshold range of 0.4-0.9, with a stable and smooth behavior (FIG. 3C), peaking at an AUC of 0.81 and 95% confidence interval of [0.587, 0.924]. Thus, for these samples the threshold could be set slightly lower, at about 0.59, although this difference may be negligible. This result is inferior to the behavior observed from the same model configuration using “O” data (about 1 standard deviation lower), which is not unexpected due to the considerably larger number of proteins and the small data set size.

The peak model performance for “S” when restricting the predictor to the 200 proteins was an AUC=0.87 and 95% confidence interval of [0.597, 0.992] (FIG. 3C). The threshold was thus essentially the same as found for the “S” analysis, and represents a considerable improvement compared to the full protein set configuration, consistent with the lowering of the false detection rate imposed by this configuration. Still, the performance of the 200 proteins configuration for “S” is slightly lower than the same configuration using “O”; however, the statistical significance of the difference (<0.3 standard deviations) is low.

### Response Prediction by RAP Number

The RAP score described above enables identifying patient-specific proteins with expression levels that correspond with non-responsiveness, as reflected by responder and non-responder expression. It was therefore hypothesized that the number of RAPs possessed by a certain patient will predict the patient's response; a patient with a small number of RAPs or no RAPs at all is expected to respond to the treatment, since almost all the measured proteins demonstrate expression levels that fit the responder population. A patient with a larger number of RAPs is expected to develop resistance since the expression level of several proteins is similar to the non-responder population. This method does not take into consideration the nature of the RAPs, and each subject may have completely different RAPs. Rather, in some cases, it is the total number of RAPs and not the identity of the RAPs that is important.

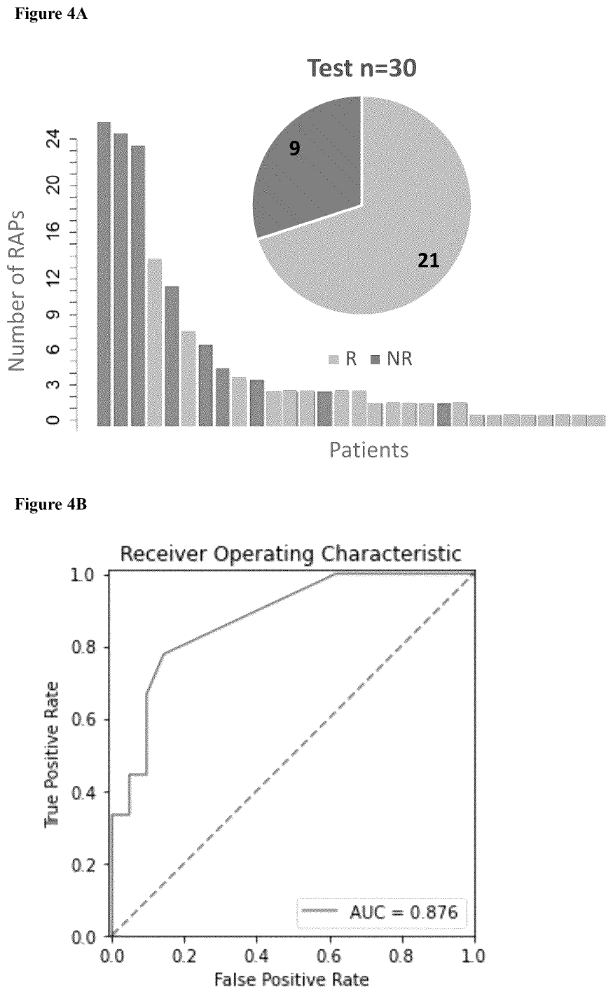

The RAP score predictive performance was tested using the test set. Specifically, for each patient in the test set (n=30), RAP score was calculated for all features using the R and NR protein level distributions of all the patients in the training set (n=78). Together with the threshold, that was calculated using the training set as explained above, it is possible to infer the number and identity of each patient's RAPs in the test set. FIG. 4A shows the 30 subjects from the test set and the calculated number of RAPs (using the mathematical method) for each subject using the T0 and T1 data. The threshold was set at 3 RAPs, and subjects with more than 3 RAPs were predicted to be non-responders. The ROC curve shows an AUC of 0.88 indicating that the analysis is highly predictive (FIG. 4B).

### Targeting RAPs

Improved understanding of molecular and immunologic mechanisms of resistance to ICI therapy may not only identify novel predictive biomarkers but may also suggest targets for combined ICI therapy. Combined therapies aim to selectively block ICI resistance proteins to improve ICI outcomes in non-responding patients.

In order to find targets for combined therapy, all RAPs with a score>2.9 (the defined threshold) found in the test set patients were evaluated. Next, a search for clinical trials in which RAPs from this list are targeted in combination with ICI in non-small cell lung cancer (NSCLC) patients or patients with solid tumors were examined. Mapping of clinical trials with combined therapy yielded 1300 clinical trials targeting 430 proteins in combination with ICI in NSCLC or solid tumors or by 500 different drugs. Comparing the 30 RAPs that passed the score threshold in the test set (RAPs appearing in at least one patient among the thirty patients and having score higher than 2.9) and the list of proteins found to be targeted in clinical trials in combination with ICI, revealed four RAPs that were also targeted in combination with ICI in NSCLC trials: KDR (VEGFR2), IL6, EPHA2 and TACSD2.

IL-6 is one of the targetable RAPs identified in the test set cohort of patients. Recently the inventors showed that therapeutic efficacy of anti-CTLA-4 is significantly improved by the coadministration of anti-IL-6 in tumor-bearing mice (Khononov, et al., 2021, “Host response to immune checkpoint inhibitors contributes to tumor aggressiveness”, J. Immunother. Cancer, March; 9). These results are in line with a previous publication demonstrating improved therapeutic outcome when anti-IL-6 is combined with anti-PD1 or anti-PD-L1 treatment. Moreover, the in vitro experiments in Khononov et al., demonstrate that inhibiting IL-6 diminishes anti-PD-1-induced tumor cell invasive properties, further supporting the notion that blocking specific therapy-induced host factors represents a strategy for overcoming therapy resistance.

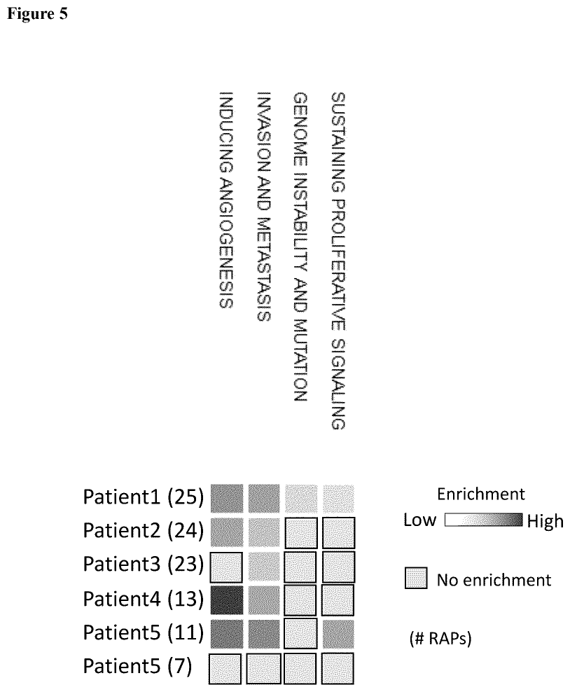

An alternative approach for therapeutic targeting based on the RAPs is by associating the proteins to main biological processes that are cancer related. To this end, each protein was assigned to hallmark/s of cancer, which capture major tumorigenic processes. Then, enrichment analysis was performed for each patient using the RAPs as an input (Fisher exact test; FIG. 5). A preliminary analysis on six patients revealed four enriched processes in total. One patient had significant enrichment in all four processes; four patients displayed enrichment of 1-3 processes; one patient did not have any significant processes.

Once the enrichment analysis is done for a patient, the treating physician can choose a therapy based on the enriched biological processes. For example, if angiogenesis is significantly enriched, the physician may choose to combine an approved drug targeting angiogenesis (e.g., Avastin) with the ICI. Another example is a patient with high proliferation signal; in this case, the physician may choose to combine ICI with a chemotherapy against tumor cell proliferation.

In order to further examine the biological aspects of the RAPs, the 19 RAPs that were obtained in at least 3 patients of the test set cohort were examined. Most patients had 4-5 RAPs. The most common RAP among the examined patients was VEGFR2 (KDR; was identified as a RAP in 12 patients). Notably, most of the RAPs were identified in T1, suggesting that resistance to therapy is mainly acquired and results from host response. VEGFR2 was identified as a RAP at both T0 and T1, though at T1 it was defined as a RAP in more patients (12 patients compared to 8 at TO). VEGFR2 is one of the two receptors of vascular endothelial growth factor (VEGF), a major growth factor for endothelial cells whose expression is higher in responders.

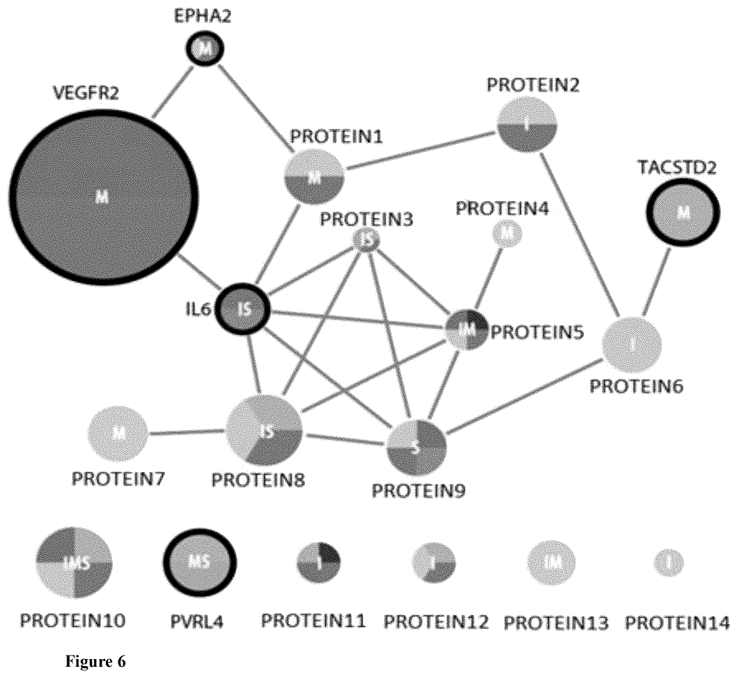

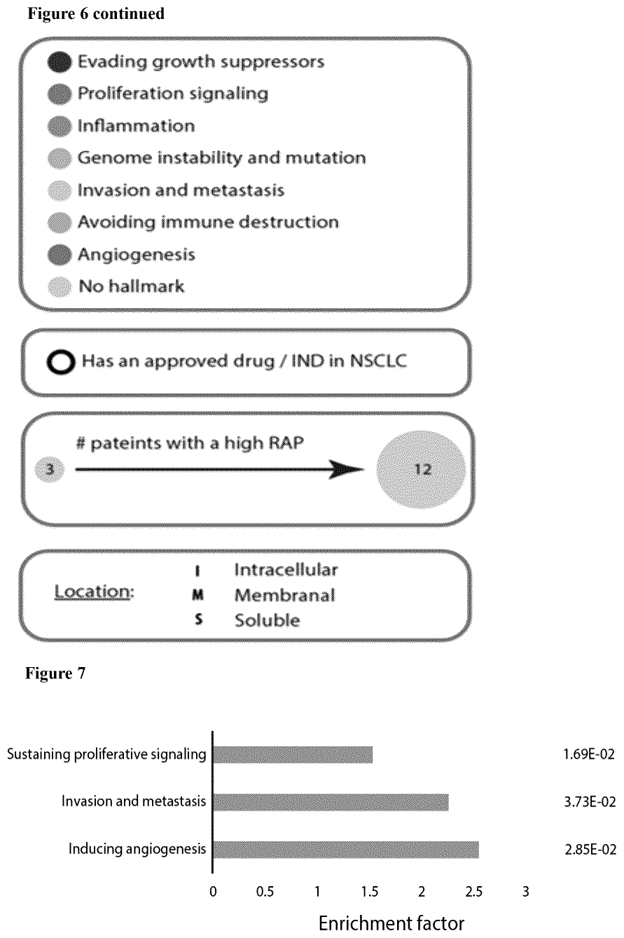

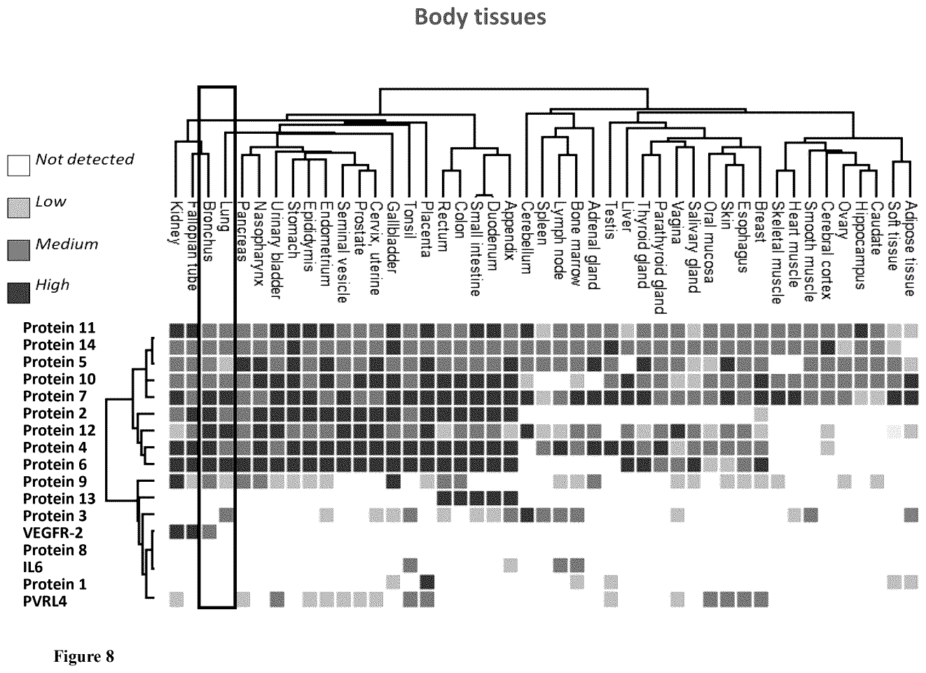

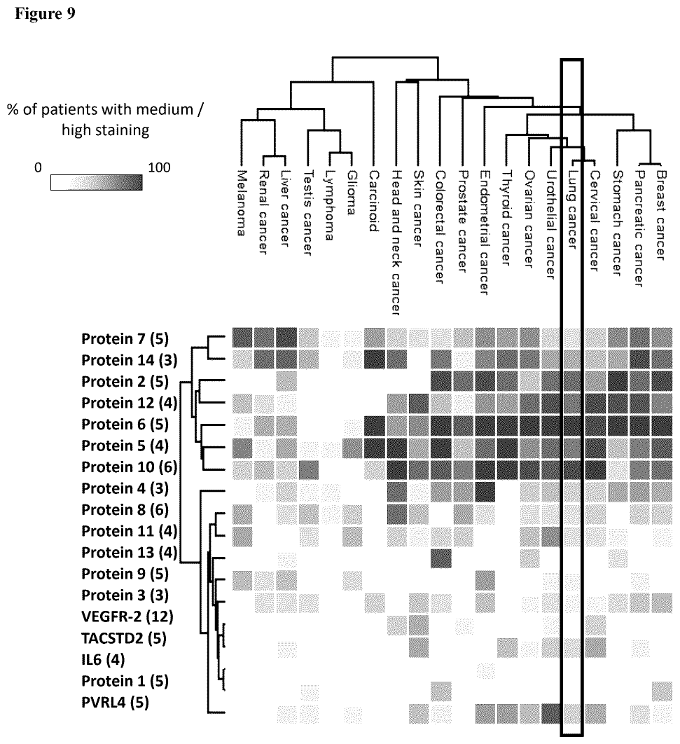

A network analysis revealed that most of the RAPs are functionally associated with each other, and five of them are highly interconnected (FIG. 6). Most proteins are associated with at least one hallmark of cancer, which further implies that these RAPs are indeed associated with resistance to therapy. Several hallmarks of cancer were significantly enriched with the 19 RAPs (FIG. 7), and multiple intracellular and membranal proteins were identified as RAPs (FIG. 6); therefore, an analysis of presumed cell of origin was performed to further understand the results (FIG. 8). Enrichment for lung and bronchus as the cell type of origin was observed. Further, various cancer types were examined for expression of the 19 RAPs and enrichment for lung cancer was also observed (FIG. 9).

### Example 2: Combining RAPs and Clinical Data

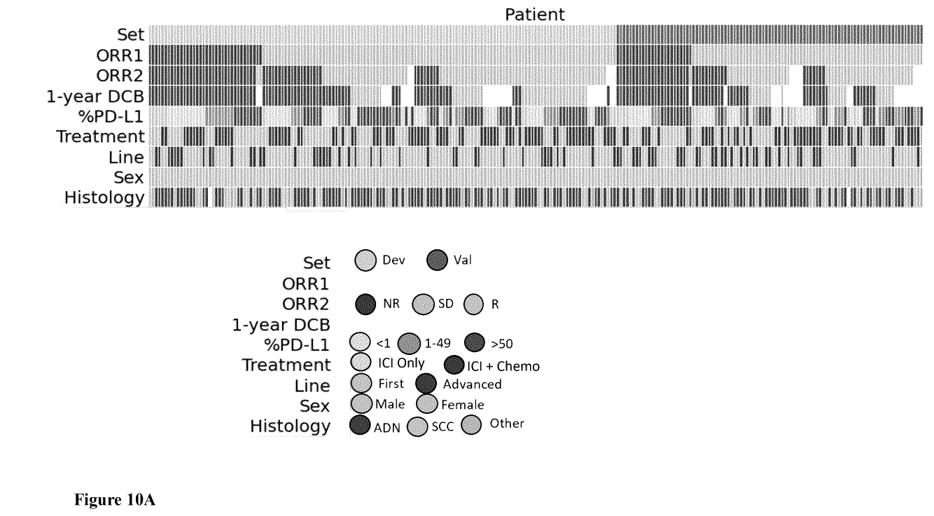

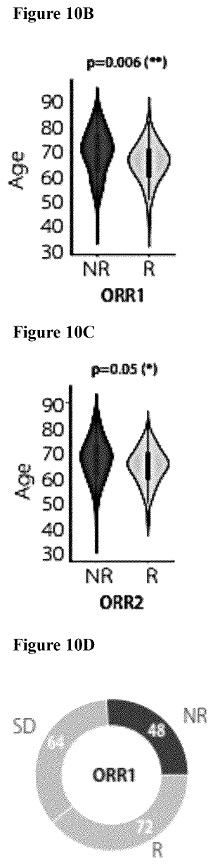

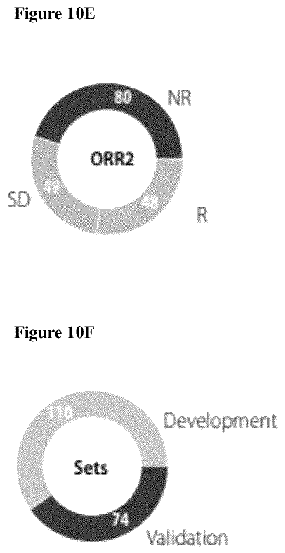

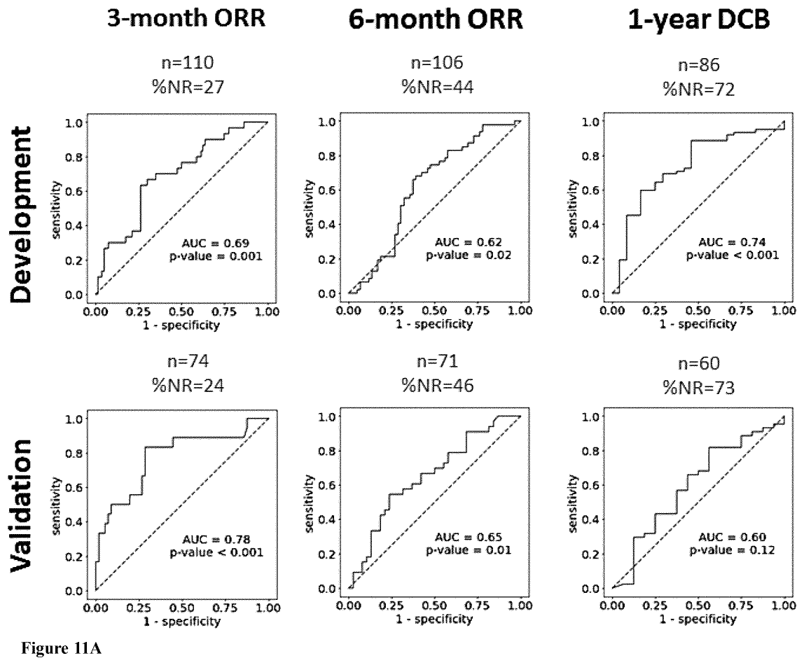

A cohort of 184 NSCLC patients was acquired from which blood samples were obtained prior to the first administration (T0) and after the first (T1) administration with ICI. Protein levels were measured. Response evaluation was based on ORR at three months and six months and durable clinical benefit (DCB) at one year post treatment initiation. Progression free survival (PFS) and overall survival (OS) were also monitored. For 3- and 6-month evaluation, subjects with progressive disease or death were considered as non-responders, while subjects with stable disease, minimal remission, partial remission, and complete remission were considered as responders. DCB was defined as one year of PFS with continued ICI treatment. Cases of ICI treatment stop due to adverse event (but no signs of progression) were treated as responders. Additional clinical information collected throughout the study included: line of treatment (first or advanced), PD-L1 immunostaining (below 1%, between 1-49%, above 50%), age and sex (see FIG. 10A-10F). The presented analysis is based on TO only. The breakdown of ICIs/therapies used is provided in Table 3.

TABLE 3

| | | Number of |
|---|---|---|
| Treatment | Target | patients |
| Pembrolizumab | PD-1 | 54 |
| Pembrolizumab, Chemotherapy | PD-1 | 86 |
| Nivolumab | PD-1 | 25 |
| Nivolumab, Chemotherapy | PD-1 | 2 |
| Ipilimumab, Nivolumab | CTLA4 | 7 |
| Ipilimumab, Nivolumab, Chemotherapy | CTLA4 | 6 |
| Atezolizumab | PD-L1 | 1 |
| Atezolizumab, Chemotherapy, targeted therapy | PD-L1 | 1 |
| Durvalumab | PD-L1 | 1 |
| Durvalumab, Chemotherapy | PD-L1 | 1 |

The cohort was divided into a development set (60% of the subjects) and a validation set (40% of the subjects). The development set was further divided into training set and test set. The models were trained on the training set and predictions were generated for a subset of patients not seen by the models during training (i.e., test sets). The division of the development set into training and test set was performed multiple times (each time for training the model on a different subset of the development set and performing predictions on the remaining patients. i.e., the training and test sets were mixed and remixed and tens of iterations were run to test that a model/classifier was effective across the entire development set) in order to generate a stable prediction for all patients in the development set. The prediction quality was then quantified by calculating the ROC AUC for the patients included in the development set. The validation set was used only at the very end of the analysis to validate the functionality of the final classifier. This division was performed multiple times,

Models were generated based on response evaluation at three time-points: three months, six months, and a year after treatment onset. All 184 patients were evaluated at the three-month time point, 177 were evaluated at six months and 146 were evaluated at 1 year. Resistance increased over time. 26% of the subject were non-responders at three months, 45% were non-responders at six months and 74% were non-responders at 1 year. These ratios were similar between the development and validation sets.

During model generation based on the development set, the development set was randomly divided into a training and a test sets 60 times. On each iteration, the top candidate proteins were selected using the Kolmogorov-Smirnov test that defines for each protein how much it differentiates between responders or non-responders. For each selected protein, a single protein XGBoost model (SP model) was generated based on the training set and predictions were made for the test set. A protein was defined as a RAP for a specific patient if the predicted resistance probability (i.e., the resistance score) was above a predefined threshold, and the average of all the iterations was used for each patient. A uniform threshold was assigned for all models, in order to handle class imbalance. Different thresholds were defined for each time point (e.g., three months threshold=0.25, six-month threshold=0.42, one year threshold=0.45). For each patient, the number of proteins for which the model score exceeded a defined threshold (i.e., the number of RAPs) was calculated.

Merely looking at the number of RAPs was predictive with this cohort. However, a predictor model was created that could also integrate clinical data. The presented clinical classifier used the number of RAPs, the line of treatment (was the ICI the first line of treatment or an advanced line), the subject's age and the percent of PD-L1 staining in the tumor (below 1% of cells positive, between 1-49%, or above 50%) as the inputs. The classifier then produced a total resistance score between 0 and 1, in which 0 was most similar to responders and 1 was most similar to non-responders. Subjects with a score above a predetermined threshold were predicted to be non-responders. Similarly, a response score, which is 1-resistance score, was also calculated. For the response score, a subject with a score above a predetermined threshold was predicted to be a responder.

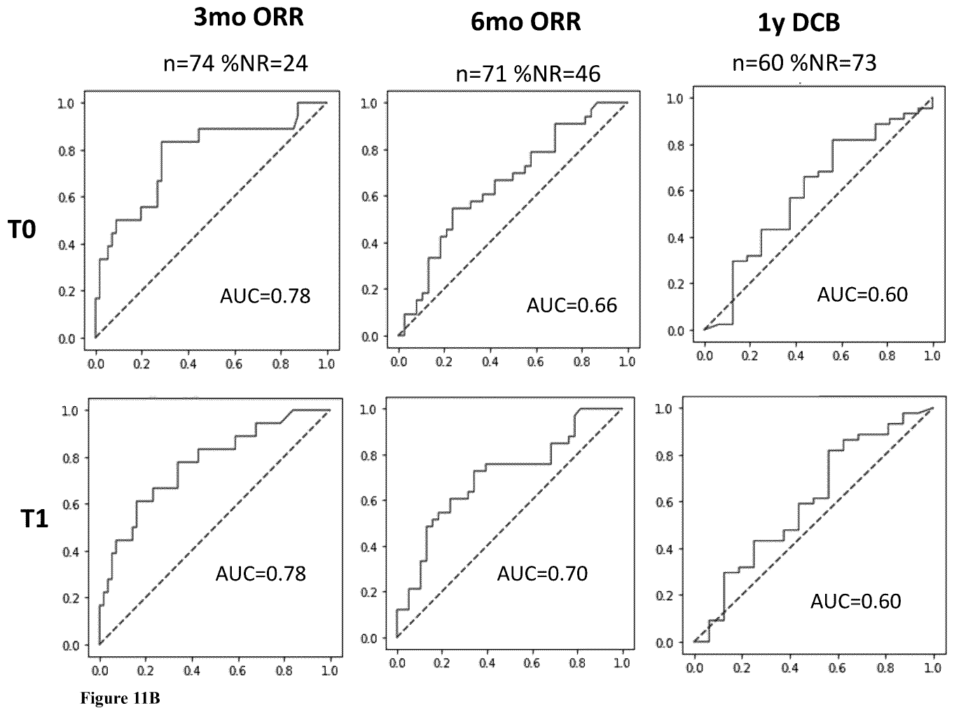

In order to test the performance of the classification model, a ROC AUC was calculated using the total resistance score together with actual response. The ROC AUC was calculated separately for 3-months ORR, 6-months ORR and 1-year DCB for both TO and T1. The results are summarized in FIG. 11A. The classifier was found to be predictive at all time points and for both development and validation sets. A similar analysis showed that the classifier was found to be predictive at all time points also for the T1 data (FIG. 11B).

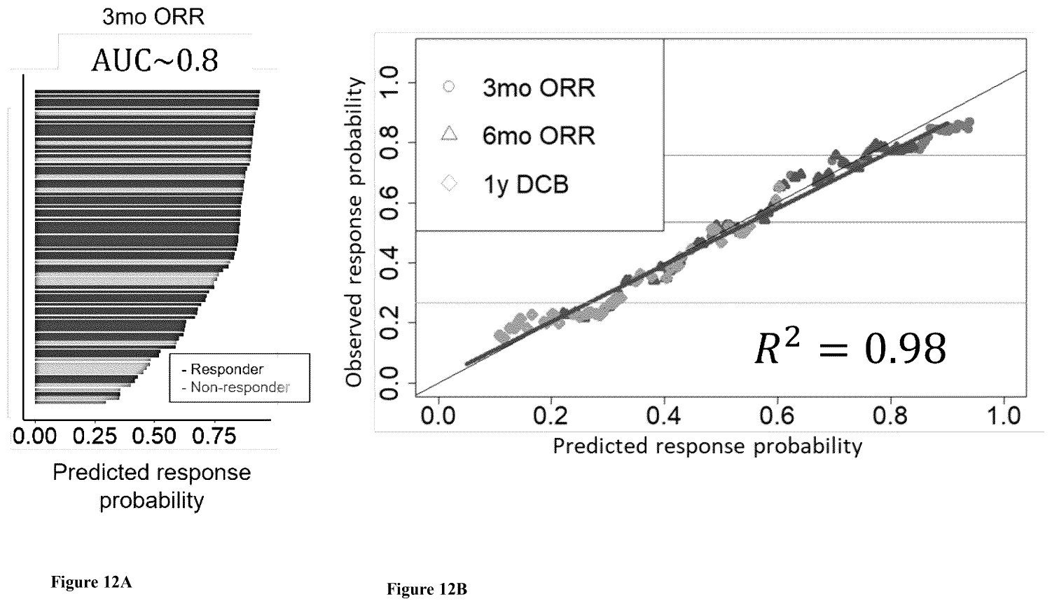

Further to checking the performance of the classification model, the correlation between the predicted response probability (response score) assigned by the classification model to each patient and the observed response probability was also examined. For this purpose, for each value of response score S₀, the observed response probability is given by the fraction of responders among patients that were assigned a response score within the range S₀+0.1. The choice of an interval of ±0.1 is arbitrary and reflects the validation set size; within a larger validation set the interval can be further reduced. The agreement between the predicted response score and the actual response probability was quantified by the goodness of fit R\hat{ }2. The goodness of fit for all 3 timepoints (3 months ORR, 6 months ORR and 1 year DCB) was R\hat{ }2=0.98 for time point TO (FIG. 12A-12B).

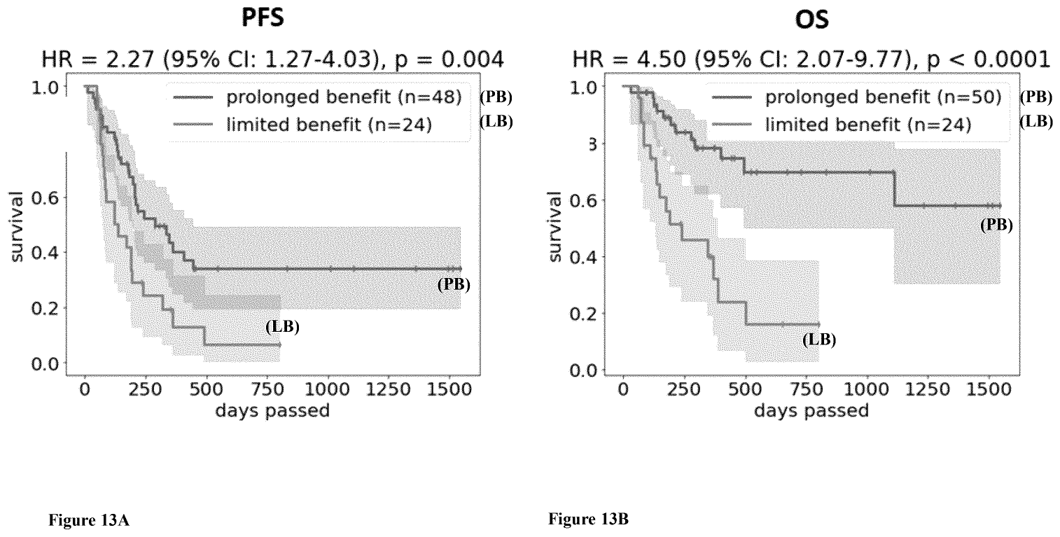

Patients within the validation set were stratified to prolonged benefit and limited benefit populations, where the stratification was based on the predicted 3-month response score. In survival analysis the stratification quality was measured by the hazard ratio (HR), which gives the ratio of probability for event per time unit within the two population. For example, HR of 4 in overall survival (OS) means that the probability for a death event per time unit among the limited benefit population is 4 times the probability per time unit among the prolong benefit population. The HR in the validation set was 2.27, p<0.004, for PFS (FIG. 13A) and 4.50, p<0.0001, for OS (FIG. 13B).

This validation experiment demonstrates that the classifier that incorporates clinical data and RAP number is highly predictive of patient response.

### Functional Network Analysis of RAPs

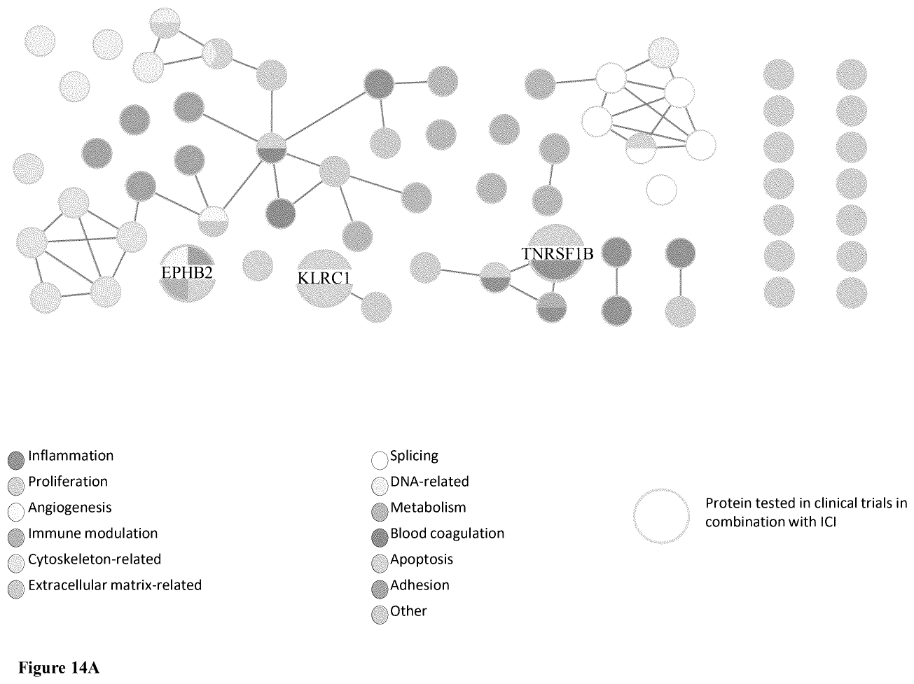

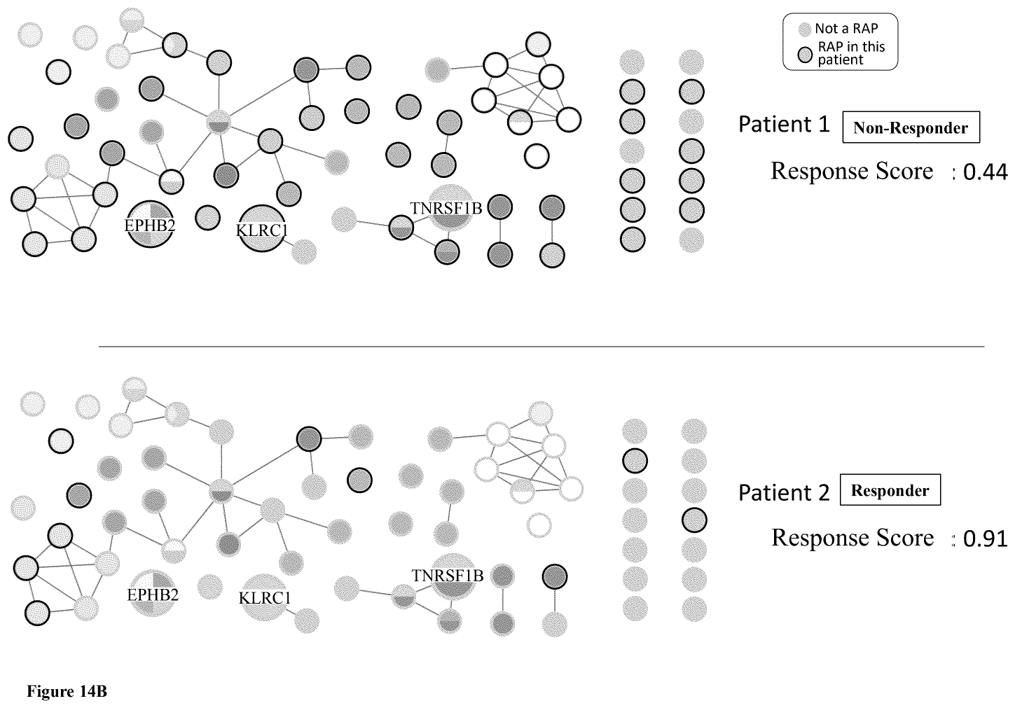

The RAP-based analysis is further used as a basis for the generation of resistance maps (FIG. 14A). The resistance map displays both the interactions between RAPs and the RAP functions. For this purpose, a RAP was defined when a protein was selected in at least 10 model iterations in one or more patients (during the RAP calculations, the model runs 60 iterations, and the number that a given protein is selected for the model is recorded), resulting in a total of 73 RAPs in the current cohort of patients. Each node represents a RAP, and the edge between nodes indicates a functional relation. Nodes with a larger size indicate investigational new drugs (INDs) in combination with immunotherapy. The nodes are colored based on the protein function. The map shows multiple interactions between different RAPs, while the RAPs are involved in different functional processes that may be relevant for resistance to therapy, such as splicing, immune modulation, angiogenesis and cell proliferation. A patient-specific map can be generated based on the patient's RAPs, which aids in 1) mapping resistance mechanisms in the individual patient and 2) identifying targeted treatments that counteract resistance. Two examples of patients in the cohort are illustrated in FIG. 14B. In these examples, a non-responder had 44 RAPs and a response probability score of 0.44 (which corresponds to a resistance score of 0.56 which is above the predetermined threshold of 0.2 for non-response). This patient had RAPs from multiple functional groups, but DNA-related RAPs were not present in this patient. The second subject was a responder with 10 RAPs, below the predetermined threshold. These RAPs were mainly related to the cytoskeleton. This patient had a high response probability of 0.91 (which corresponds to a resistance score of 0.09 which is below the predetermined threshold of 0.2.

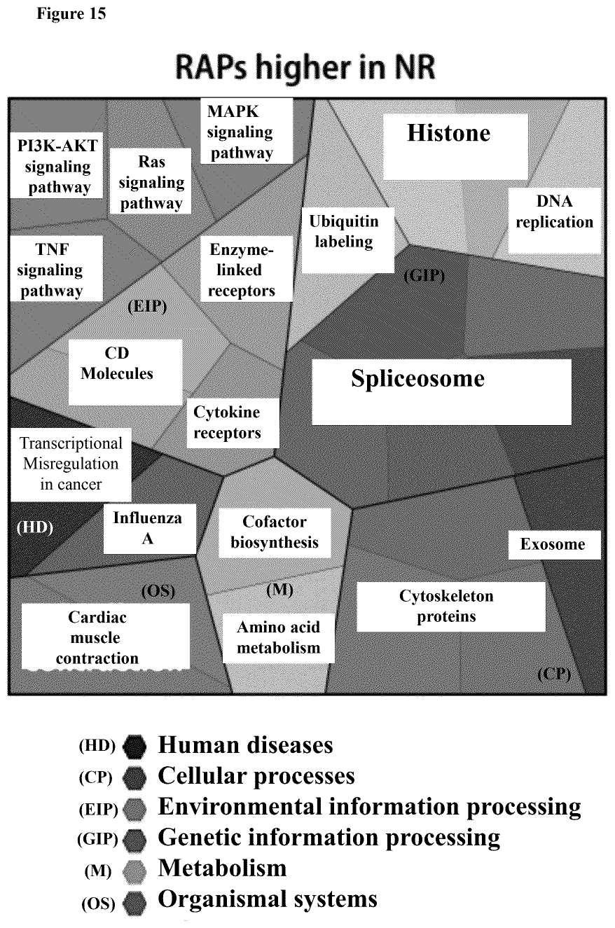

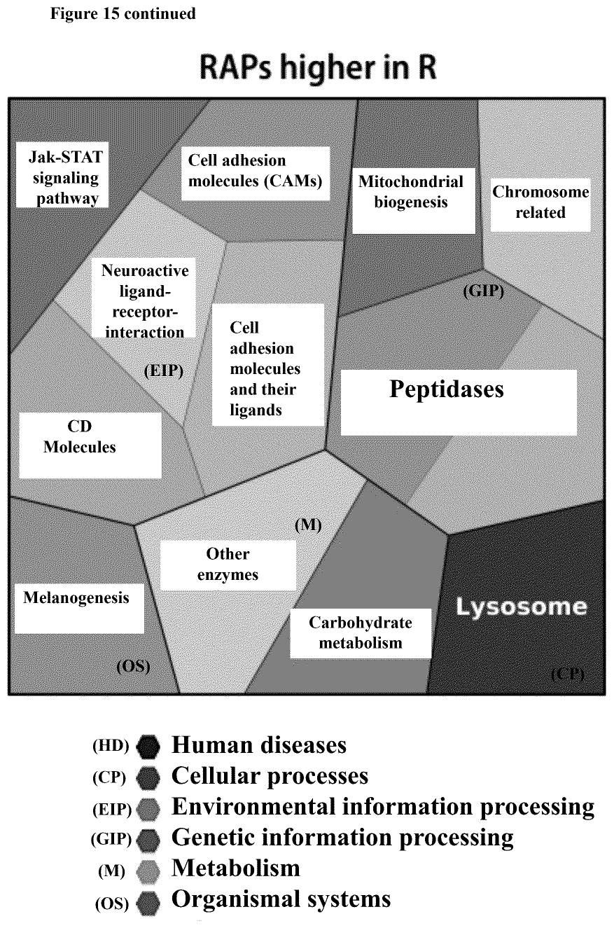

Further examination of the patient RAPs shows functional differences between RAPs with higher representation in each response group (FIG. 15). While non-responder RAPs are involved in splicing, signaling and cytoskeleton-related processes, the responder RAPs are mainly involved in proteolysis and cell adhesion. Interestingly, RAPs higher in the responder group includes 2 peptidases that may be involved in antigen presentation, thereby promoting response to therapy. In order to convert non-responders to responders a RAP is selected for which there is a known therapeutic agent. The agent is selected such that it modulates the RAP to alter pathway function to more closely approximate pathway function in responders. If therapeutics that target the RAPs are unavailable or undesirable a therapeutic agent that modulates the pathway containing the RAP is selected. The selected agent must modulate the pathway containing the RAP to alter pathway function so that it more closely approximates pathway function in responders. The therapeutic agent is used to convert non-responders to responders or as a combination treatment with the ICI.

### Example 3: The RAP-Based Model Forecasts Differential Outcomes Based on PD-L1-Tumor Expression in Patients

To develop a blood-based model for predicting benefit from first-line PD-(L)1-based ICI therapy, blood plasma samples and clinical data were collected from ICI-treated, advanced stage NSCLC patients. Pre-treatment plasma samples from 425 patients were profiled by a protein assay that measures approximately 7000 proteins in a single plasma sample. Following patient exclusion due to technical or clinical reasons, the study cohort consisted of 339 remaining patients.

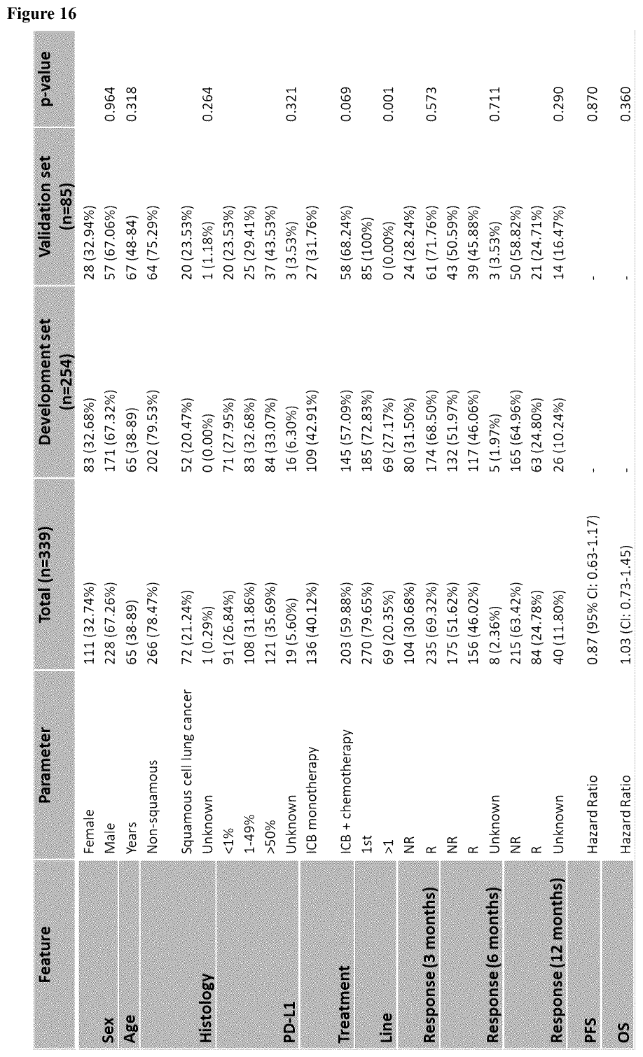

Patient clinical parameters are presented in FIG. 16. The median age was 65 years with a predominance of male patients as a third of the patients were female. The majority of patients (78.47%) had non-squamous cell carcinoma (mostly adenocarcinoma) and 21.24% of the patients had squamous cell carcinoma, in agreement with expected proportions. Most of the patients had ECOG performance status of 0-1 (94%). Patients were either treated with ICI-chemotherapy combinations (59.8860%) or ICI monotherapy (40.12%). There was an approximately equal distribution of patients with PD-L1-negative, PD-L1-low and PD-L1-high tumors, where negative, low and high refer to PD-L1 expression on <1%, 1-49% and ≥50% of the tumor cells, respectively. The PD-L1-high group was the largest (36%). Clinical benefit (CB) was defined as previously described.

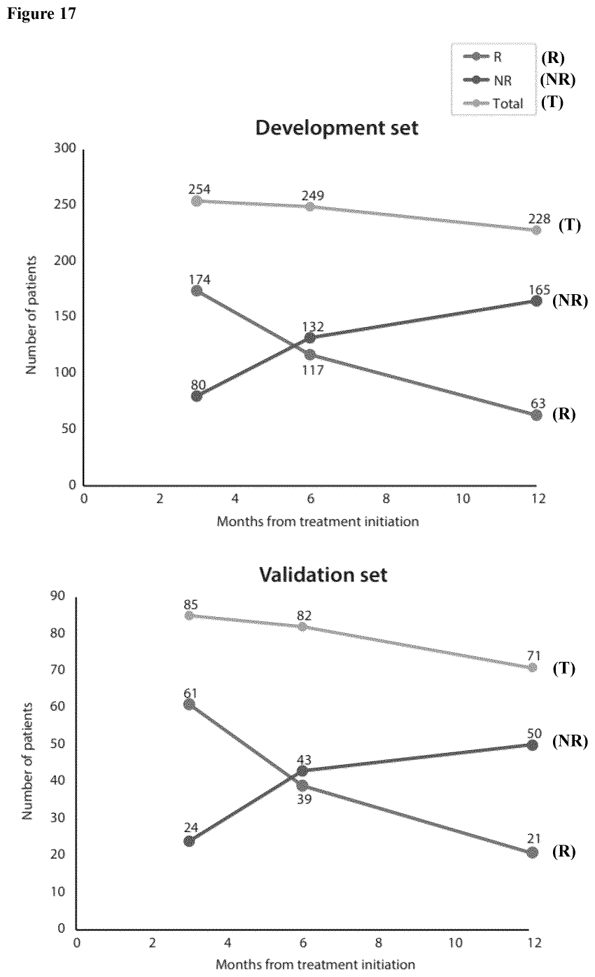

Therapeutic benefit was assessed at 3, 6 and 12 months after commencement of treatment. For each time point, patients were categorized into clinical benefit (CB), or no clinical benefit (NCB) groups as follows. At the 3- and 6-month time points, patients displaying complete response, partial response or stable disease were classified as CB patients, whereas patients displaying progressive disease or who had died were classified as NCB patients. At the 12-month time point, patients who were alive and displayed durable clinical benefit (defined as absence of progressive disease for at least 1 year after starting treatment) were classified as CB patients, and all other patients were classified as NCB patients. Based on these criteria, 69.32%, 46.02% and 24.78% of the patients achieved CB at 3, 6 and 12 months, respectively (FIG. 16). The cohort size varied between time points due to patient death or lack of clinical benefit data per time point (FIG. 17). As such, the dataset included 339, 331 and 299 patients for the 3-, 6- and 12-month time points, respectively.

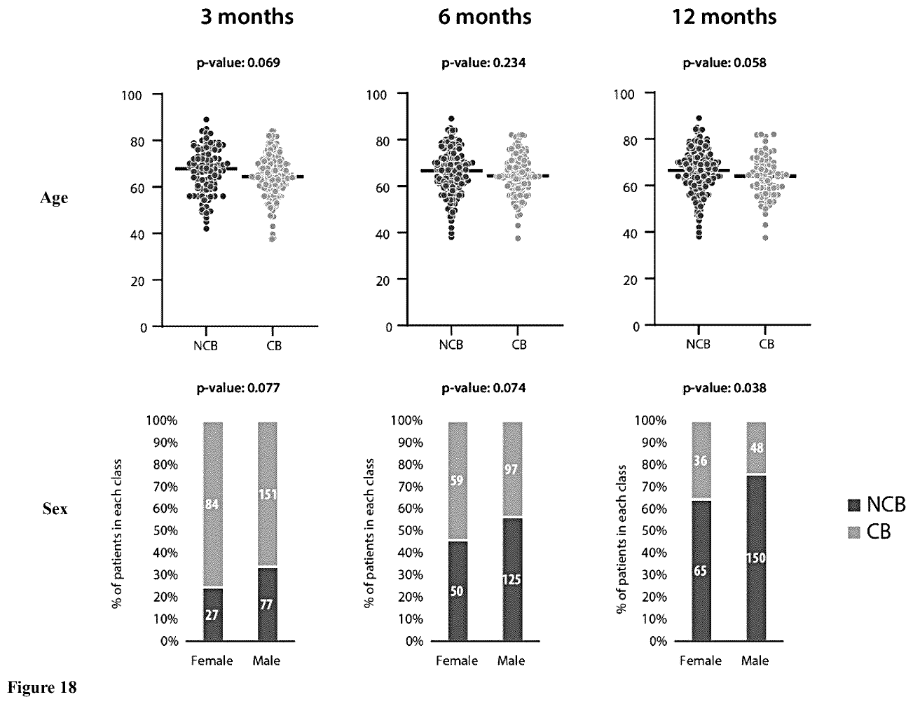

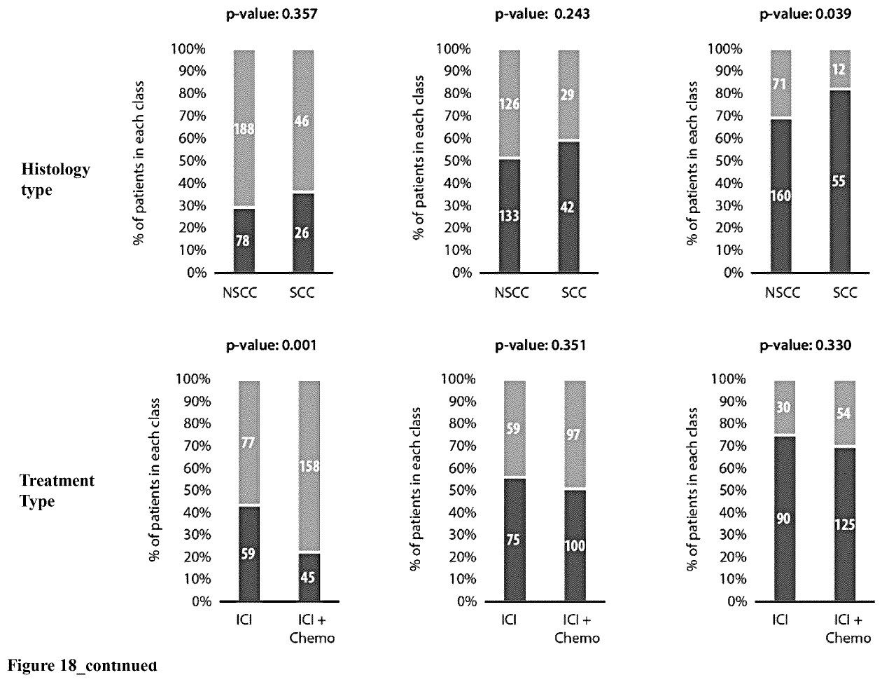

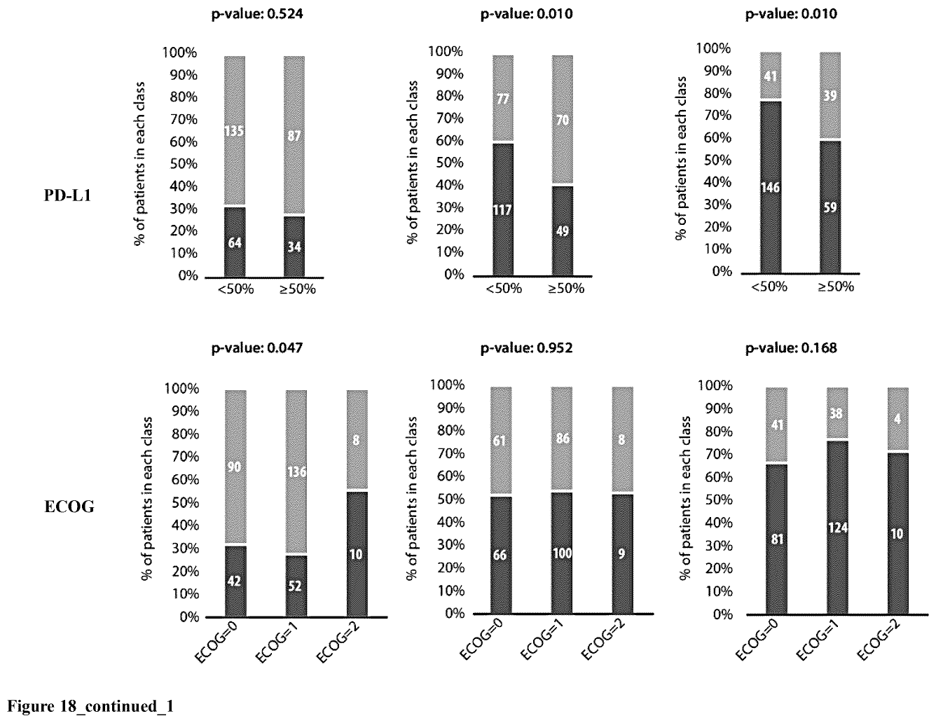

Various clinical parameters were found to be associated with CB (FIG. 18). At the 3-month time point, a higher proportion of CB patients was found in the ICI-chemotherapy-treated group in comparison to the ICI monotherapy group (78% vs. 57%, respectively; p-value=0.001), while no associations between treatment type and CB were found at the other time points. PD-L1 status correlated with CB at the 6- and 12-month time points, as a higher proportion of CB patients was found in the PD-L1-high group in comparison to the combined group of PD-L1-low and PD-L1-negative patients (66% vs. 59%, respectively, for 6 months; p-value=0.010; 40% vs. 22%, respectively, for 12 months; p-value=0.010). In addition, at the 12-month time point, the non-squamous lung cancer group had a higher CB rate in comparison to the squamous cell carcinoma group (31% vs. 18%, respectively; p-value=0.039). ECOG performance status correlated with CB at the 3-month time point, with a higher proportion of CB patients in the ECOG 0 and 1 groups compared with ECOG 2 (68% and 72% vs. 44%, respectively; p-value=0.047). Finally, a higher CB rate was found in females in comparison to males at 12 months (36% vs. 24%, respectively; p-value 0.038).

### Example 4: Predicting Benefit from ICI Therapy Based on Clinical Parameters

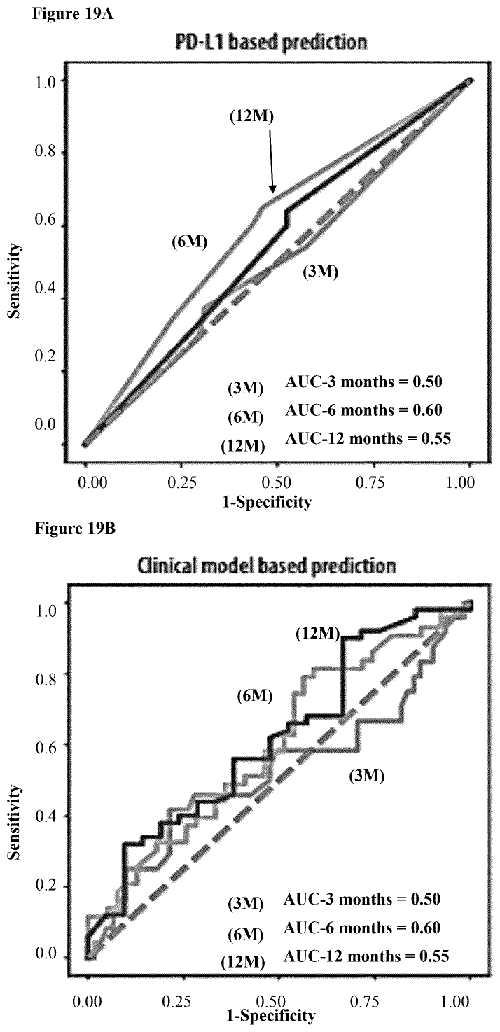

While PD-L1-based companion diagnostic tests recommend the use of ICI monotherapy for PD-L1-high NSCLC patients, clinical evidence also demonstrates a trend for increased benefit with increasing tumor PD-L1 levels in patients treated with combination ICI-chemotherapy. Evaluating the predictive performance of the PD-L1 biomarker was performed over a range of expression levels (i.e., <1%, 1-49% and ≥50%) in the mixed cohort comprised of patients treated with either ICI monotherapy or combination ICI-chemotherapy. Predictive models were generated for each CB assessment time point (3, 6 and 12 months) with a division of the cohort into development and validation sets. The development set, comprised of 75% of the patients (n=254), was used for model generation. Once the model was developed, the overall performance was assessed in a blinded manner on the independent validation set comprised of the remaining 25% of the patients (n=85; FIG. 19A).

Even though PD-L1 expression correlated with CB at the 6- and 12-month time points (p-value=0.01; FIG. 18), CB prediction at each of the three time points was poor, with area under the curve (AUC) of the receiver operating characteristics (ROC) plot of 0.50 (p-value=5.13e−01), 0.60 (p-value=6.13e−02) and 0.55 (p-value=2.76e−01) at 3, 6 and 12 months, respectively (FIG. 19A).

We next asked whether integrating additional clinical parameters would improve the predictive capability of the PD-L1 biomarker. Three clinical parameters known to correlate with treatment benefit, namely, patient sex, ECOG performance status, and line of treatment, were considered. Accordingly, we developed a predictive model based on PD-L1, sex, ECOG and treatment line, termed here as the ‘clinical model’. The clinical model displayed only a minor improvement in response prediction capability compared to PD-L1 alone, with AUCs of 0.52, 0.60 and 0.62 for 3, 6, and 12 months, respectively (FIG. 19B). Therefore, a stronger predictive model is required.

### Example 5: The Resistance Associated Protein (RAP) Prediction Model

Aiming to develop a more robust predictive model, we designed an additive model where the output is based on the sum of predictions from a large collection of individual features associated with therapeutic benefit. Since each feature on its own has a minor effect on the final output, the effects of any false discoveries are minimized, and model stability is maintained. This approach potentially mitigates the effects of significant heterogeneity between patients and the large number of features in a comparatively small cohort.

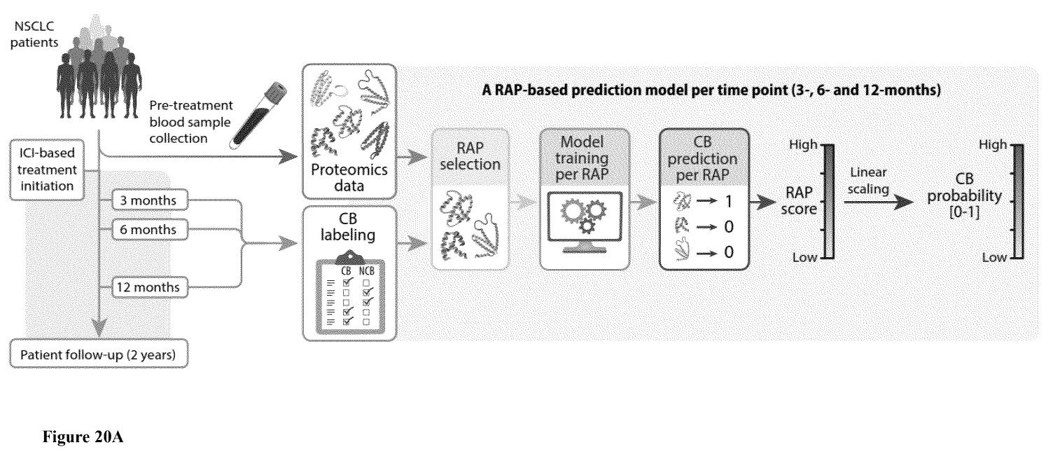

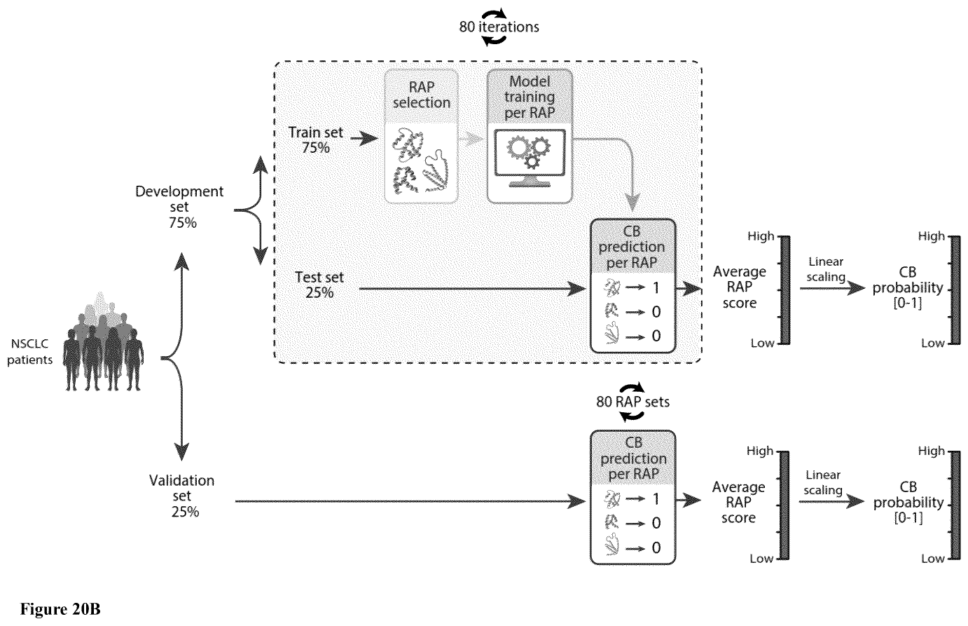

Briefly, the model is based on a set of proteins that display differential plasma level distributions in CB and NCB populations, as determined by a statistical test. Such proteins, termed resistance associated proteins (RAPs), serve as potential indicators of treatment benefit depending on their plasma level in the individual patient (FIG. 20A). Specifically, for a given patient, a machine learning (ML)-based model that was trained on CB and NCB populations infers a CB or NCB prediction from the plasma level of each one of the patient's RAPs within the entire RAP set. In this way, the patient is assigned a collection of predictions based on his/her personal RAP profile, and the sum of all predictions reflects the patient's likelihood of benefiting from treatment. Patients displaying numerous CB predictions are more likely to benefit, whereas patients with numerous NCB predictions are less likely to benefit.

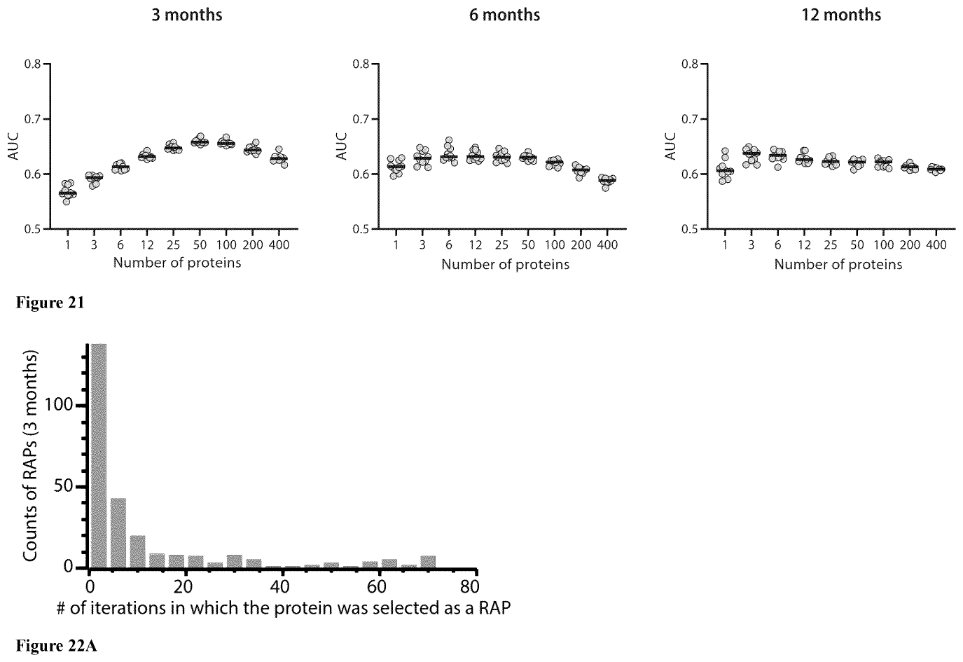

Three RAP-based models were developed, one for each of the three CB assessment time points. The models were developed following the same workflow, where CB labelling for the 3-, 6- or 12-month time points, together with protein expression data and patient sex, were used as input (FIG. 20A). Firstly, to define the collection of RAPs on which the final model will be based, the development set (75% of the patient cohort; n=254) was divided into train and test sets consisting of 75% and 25% of the development set patients, respectively. (FIG. 20B; see also Materials and Methods). Proteins displaying statistically significant differences between their plasma level distributions in CB and NCB populations were identified in the train set, and the 50 proteins with the lowest p-values were selected as RAPs (FIG. 21; see also Methods). Next, for each selected RAP, a ML algorithm was trained with two features, namely, RAP expression level and patient sex, to develop a binary classifier for therapeutic benefit per RAP. Predictions were then inferred per RAP for each patient in the test set and a RAP score was computed based on the collective predictions from the 50 selected RAPs. The 3-step process (i.e., RAP selection, model training and RAP score computation) was repeated 80 times, each time with a random division of the patients into train and test sets (FIG. 20B). RAP scores were averaged per patient and linearly scaled to generate a model whose final output is CB probability—a clinically oriented metric reflecting the patient's likelihood of benefiting from treatment.

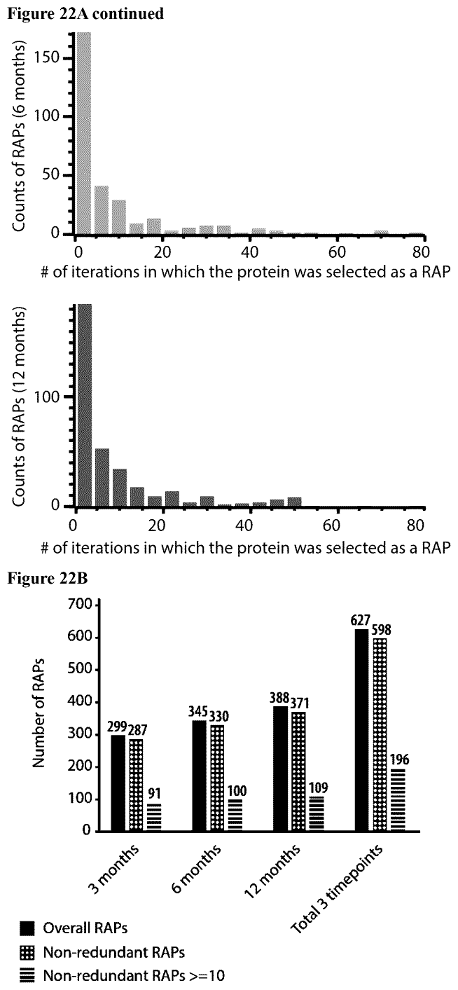

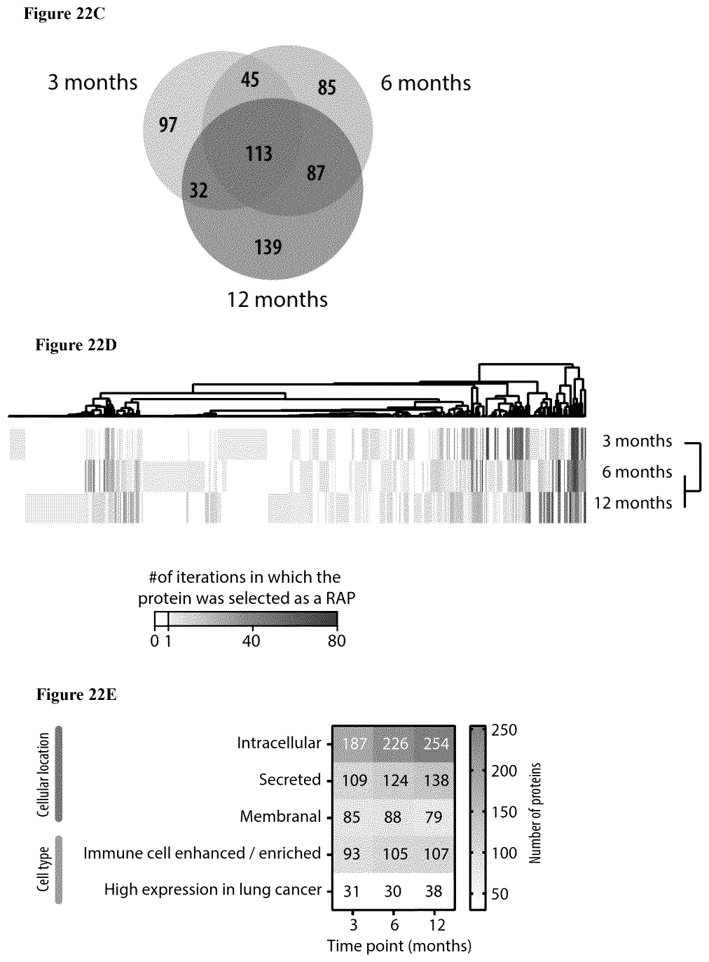

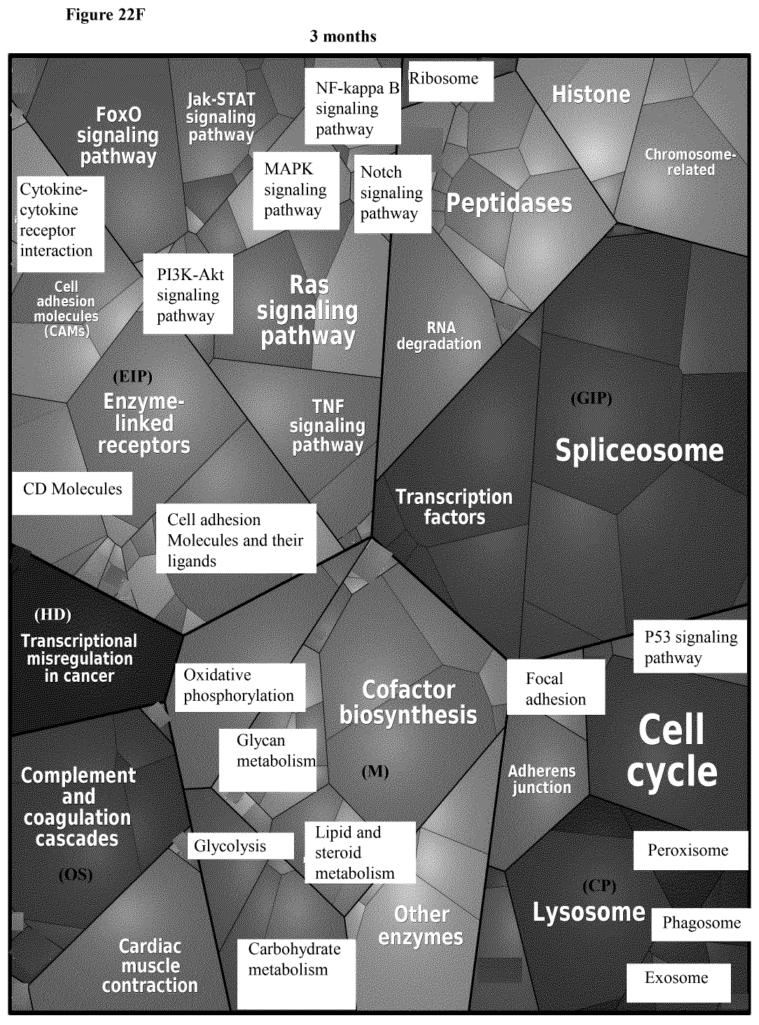

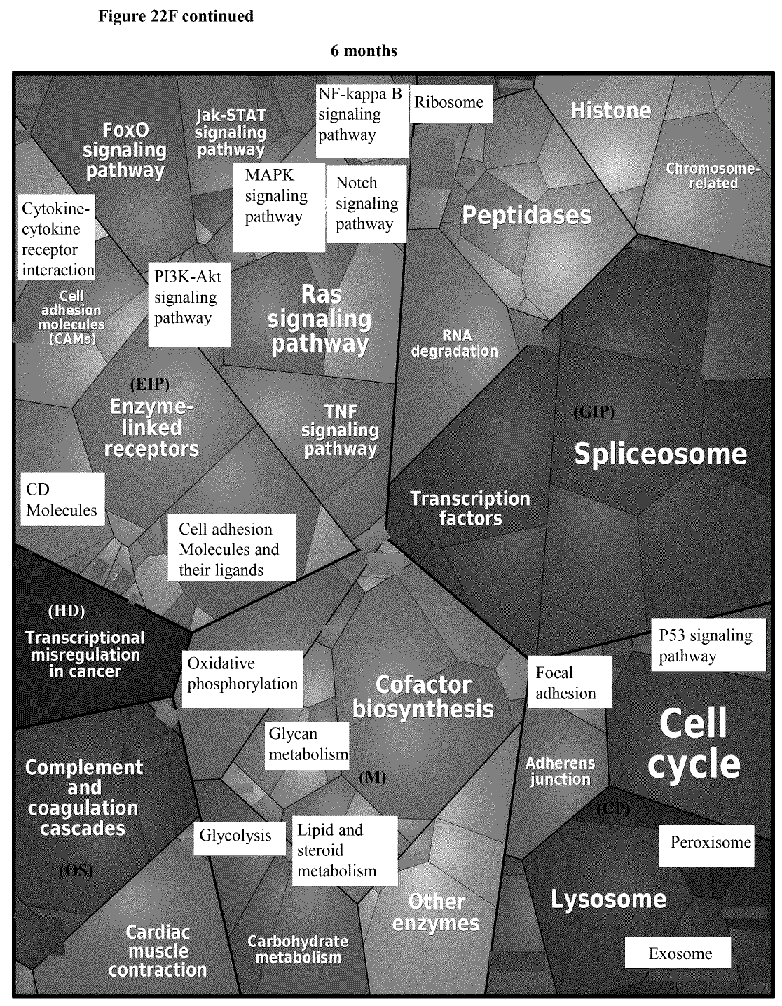

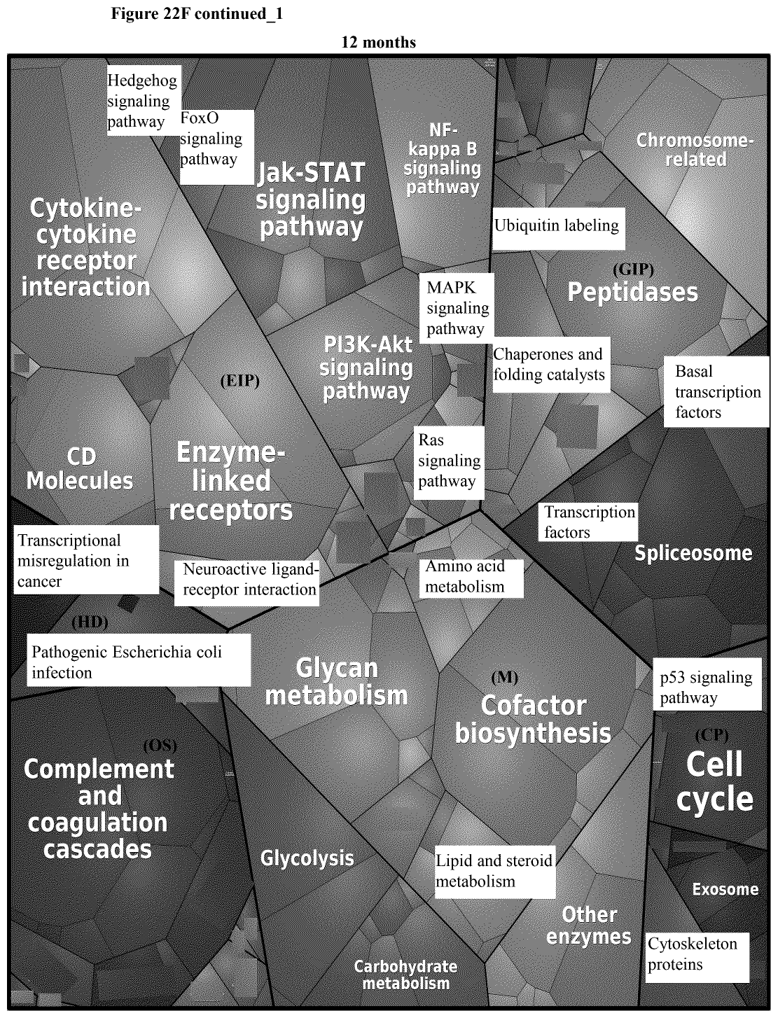

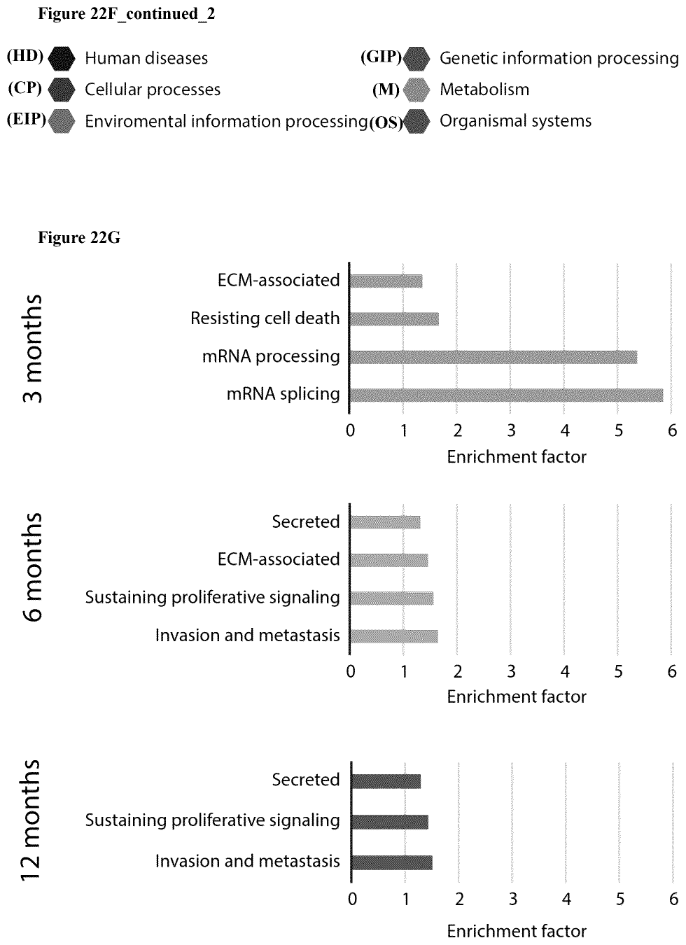

Since RAP selection was performed via an iterative process during model development (50 RAPs were selected from the train set after randomly mixing the patients between train and test sets 80 times), the same RAPs could be selected several times overall (FIG. 22A). Out of a total of 287, 330 and 371 RAPs selected for the 3-, 6- and 12-month time points, respectively, approximately 100 RAPs were selected at least 10 times per time point (FIG. 22B). Across the three time points, a total of 598 RAPs were selected, out of which 113 RAPs were common to all three time points (FIG. 22C). In addition, approximately 30 RAPs were selected more than 10 times across the three time points (FIG. 22D).

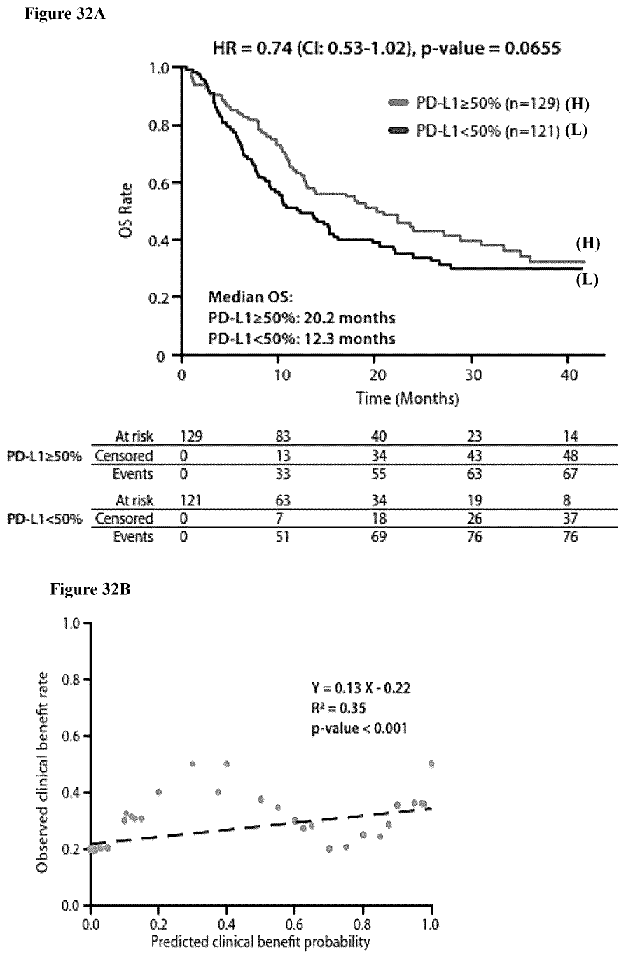

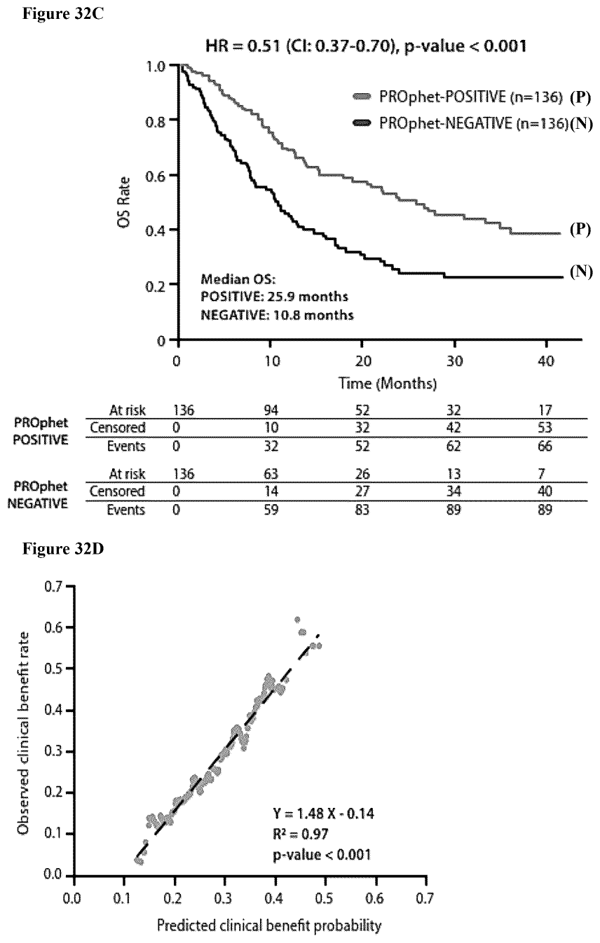

To gain insight into the biological functions of the selected RAPs, we first categorized them according to cellular location and origin based on the Human Protein Atlas database. Mostly, RAPs were found to be intracellular proteins, with a large proportion possibly originating from immune cells. Approximately 8-10% of RAPs per time point are known to be highly expressed in lung tumors (FIG. 22E). Next, we performed a functional analysis of the RAPs selected per time point. At all time points, multiple RAPs were found to be involved in splicing or alternative splicing (FIG. 22F), while splicing was significantly enriched at the 3-month time point (FIG. 22G); Fisher exact test, false discovery rate <0.1; the test was applied using a cut-off of identification in at least 10 iterations, but similar results were obtained using different cut-offs). In addition, at all three time points, multiple RAPs were associated with complement and coagulation cascades (FIG. 32F). Lastly, extracellular matrix (ECM) related pathways, represented by proteins such as Osteopontin (SPP1) and TIMP1, were significantly enriched at 3 and 6 months, whereas two hallmarks of cancer (namely, sustaining proliferative signaling and invasion and metastasis) were significantly enriched at 6 and 12 months (FIG. 22G). Notably, multiple RAPs, such as VEGFA, IL-6, FLT4, CSFIR and CA125 (MUC16) are known targets of approved and investigational therapeutic agents, some of which are being explored in combination with ICIs in clinical trials. Overall, these findings demonstrate an association between RAPs and biological pathways related to tumor progression and treatment resistance.

### Example 6: The RAP Model Predicts Benefit from ICI Therapy

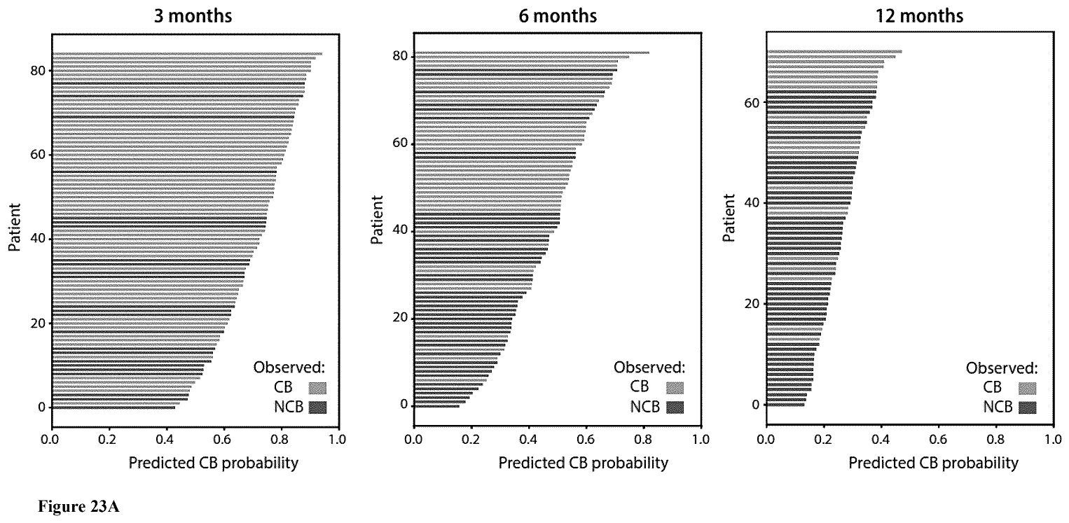

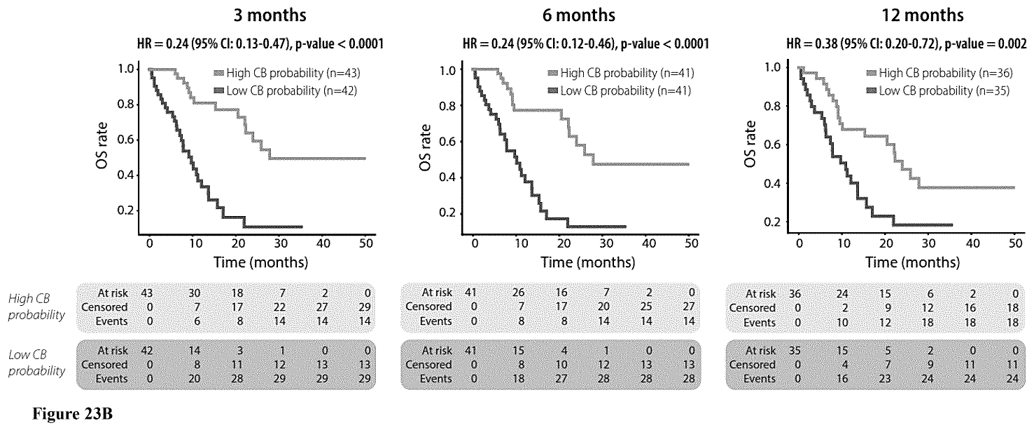

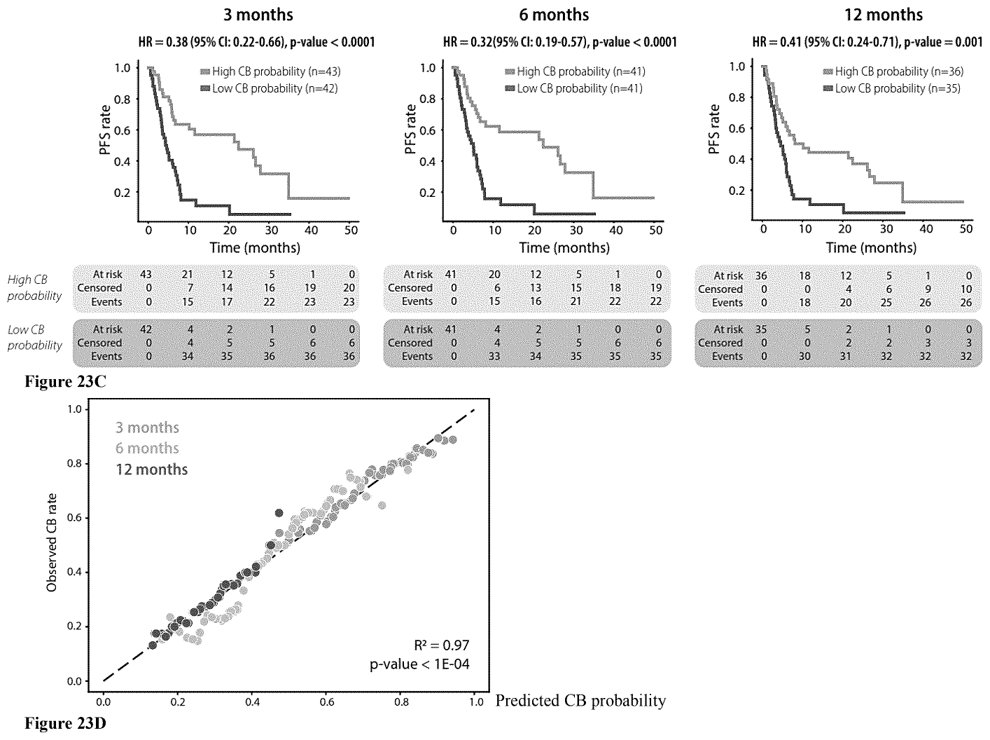

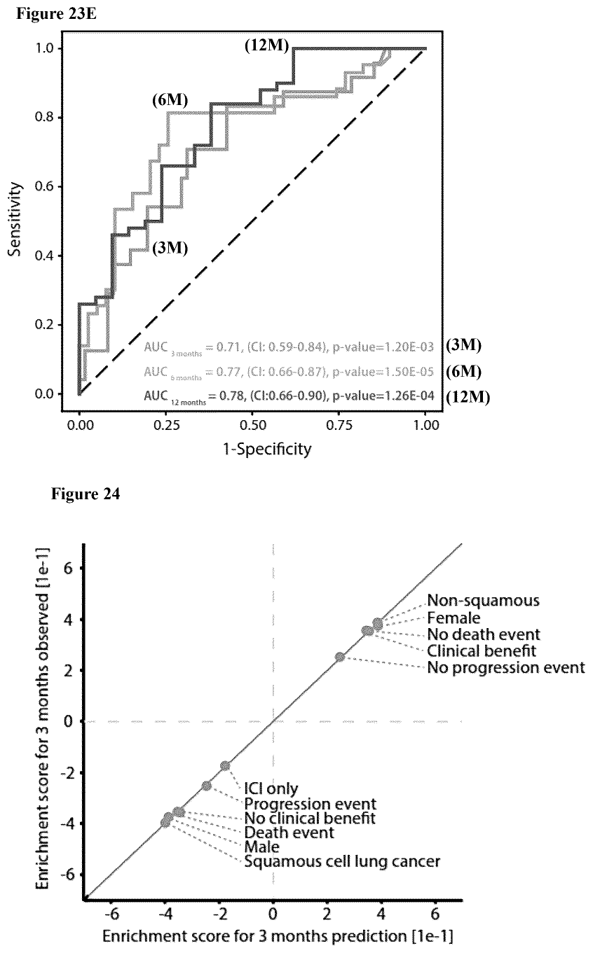

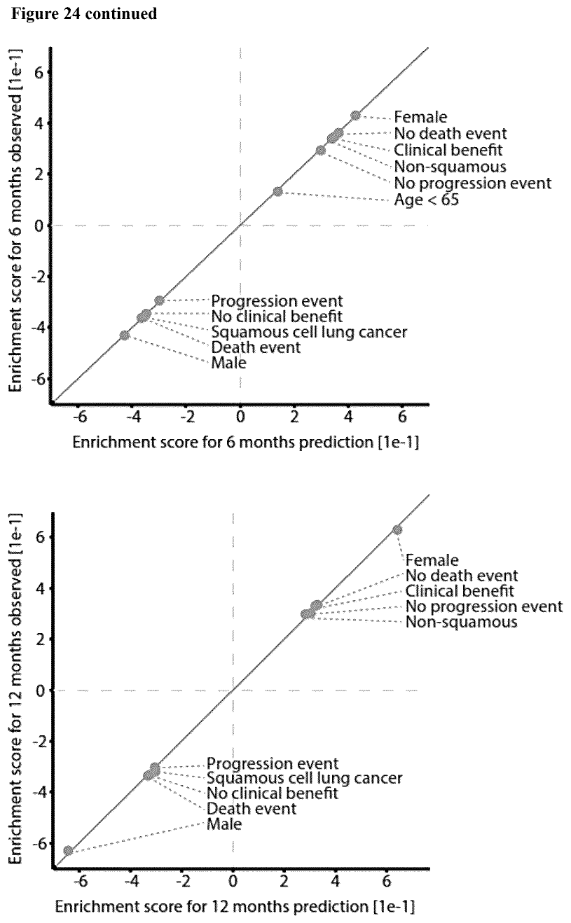

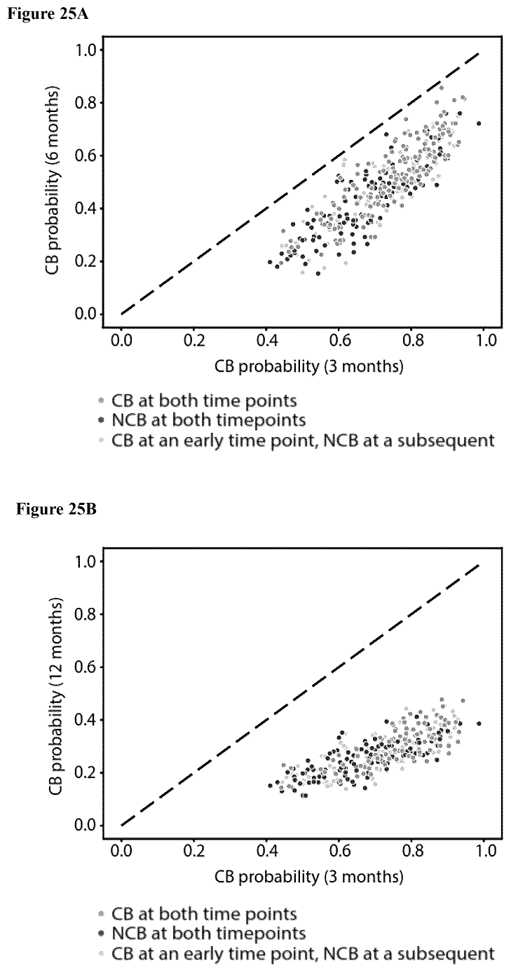

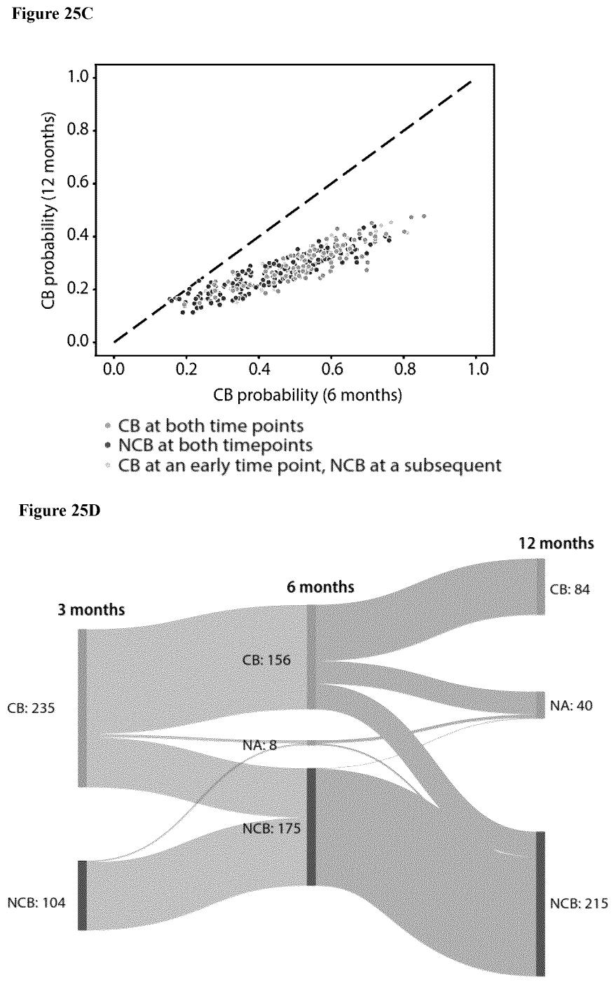

After model development, the RAP models for each time point were locked and tested in a blinded manner on the independent validation set (25% of the patient cohort; n=85). The validation set was comprised of advanced stage NSCLC patients treated with first-line PD-(L)1-based ICI therapy, cither as a monotherapy or in combination with chemotherapy. CB probabilities were determined for each patient in the validation set per time point. The range of the CB probability distribution was different for each time point, with a decrease in the median CB probability over time (FIG. 23A). In addition, the CB probabilities of all patients decreased from one time point to any subsequent time point (FIG. 25A-25C), in agreement with the actual decreased CB rate over time (FIG. 25D). Notably, actual NCB patients clustered at the lower range of predicted CB probabilities for all 3 time points, indicating that the models have high predictive power (FIG. 23A). This finding was further strengthened by an enrichment analysis based on CB probabilities (2D enrichment test; False discovery rate <0.05). Specifically, at all three time points, the group of patients with high CB probability values was significantly enriched with CB patients, females, patients with non-squamous cell carcinoma, and patients with no progressive disease or death events. On the other hand, patients with low CB probability values were significantly enriched with NCB patients, males, patients with squamous cell carcinoma and patients with progressive disease or death events (FIG. 24).

Next, using the median CB probability as a threshold, we classified the patients into high or low CB probability groups. Specifically, patients with a predicted CB probability above or below the median were assigned to high or low CB probability groups, respectively (FIG. 23B). A log-rank test demonstrated that patients in the high CB probability group achieved significantly longer overall survival (OS) than patients in the low CB probability group across the 3 time points (FIG. 23B, Hazard Ratio, HR=0.24-0.38). Similar results were obtained for progression-free survival (PFS; FIG. 23C, HR=0.32-0.41). These findings demonstrate that the RAP-based models effectively classify survival outcomes in ICI-treated NSCLC patients.

To further test model accuracy, predicted CB probability was compared to the observed CB rate, where the latter refers to the proportion of observed CB patients within the group of patients assigned a similar CB probability (i.e., CB probability ±0.15). Linear regression analysis demonstrated a high goodness of fit (R2=0.97) between predicted CB probability and observed CB rate (FIG. 23D). Additionally, the AUCs of the ROC plots were 0.71, 0.77 and 0.78 for the 3-, 6- and 12-month time points, respectively (FIG. 23E), demonstrating strong predictive capability of the RAP models over the first year of ICI-based treatment. Notably, the RAP model displayed superior predictive performance in comparison to the PD-L1-based model (AUCs=0.5-0.6 over the first year) and the clinical model (AUCs=0.52-0.62 over the first year) (FIG. 19A-19B).

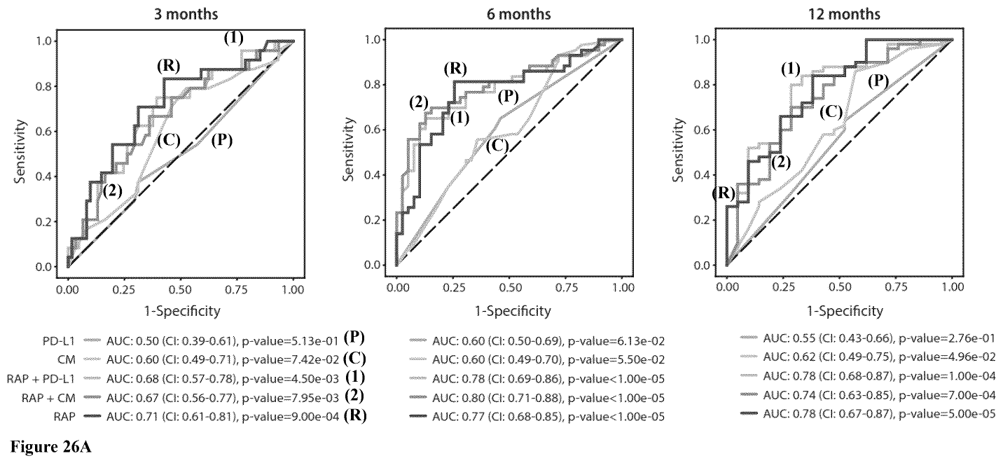

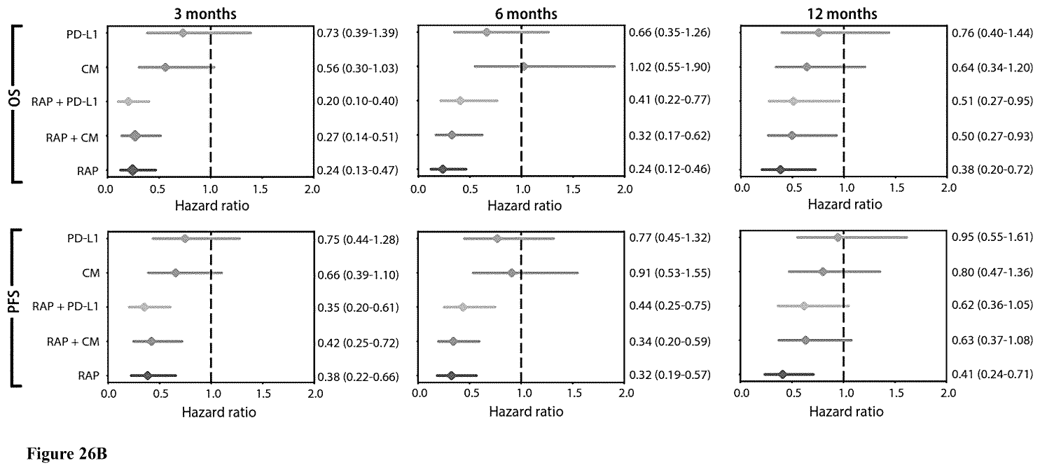

We next asked whether integrating clinical parameters into the RAP model would improve its predictive performance. To this end, we integrated the PD-L1-based model (PD-L1) or clinical model (CM) with the RAP model and compared predictive performance. Interestingly, adding the PD-L1 parameter to the RAP model slightly increased predictive performance for the 6-month time point, while integrating the RAP and clinical models decreased predictive performance overall (FIG. 26A). In the survival analysis, the RAP model displayed the best HR in comparison to the four other models, while the HR was not significant for the PD-L1-based and clinical models (FIG. 26B).

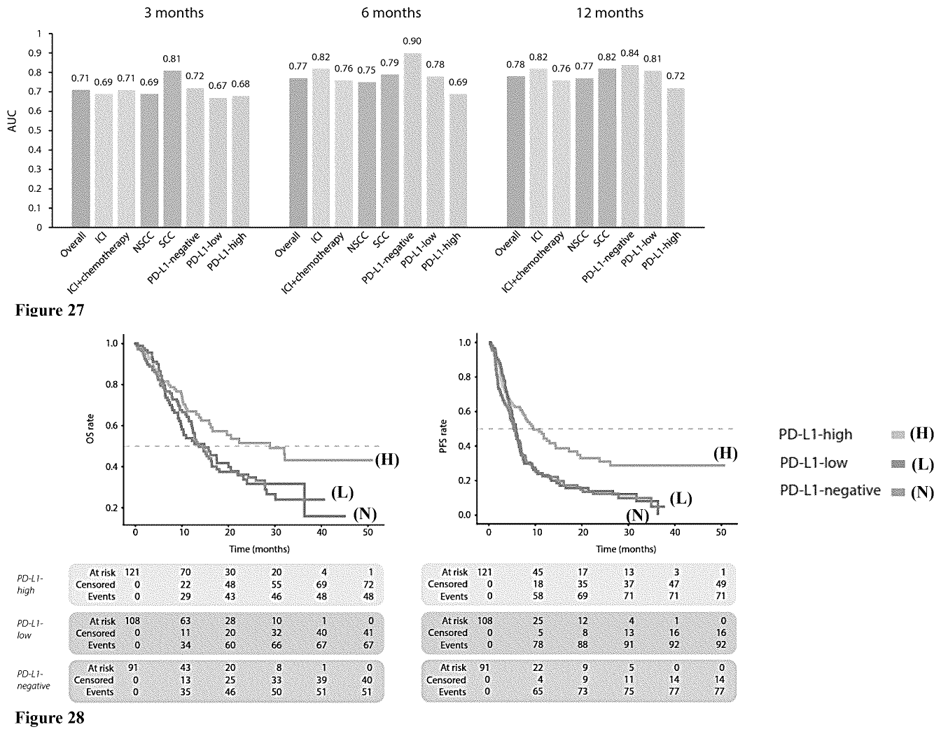

Lastly, we investigated RAP model performance in different patient subsets (FIG. 27). The model displayed strong predictive performance in both ICI monotherapy and ICI-chemotherapy subsets, similar to the performance in the population overall. Histology subset analysis, on the other hand, showed improved prediction for the squamous cell carcinoma subset at 3 months compared to the overall population. At 6 and 12 months, the strongest prediction was observed in the PD-L1-negative subset, while prediction was slightly weaker in the PD-L1-high subset compared to the overall population.

### Example 7: The RAP Model Forecasts Differential Outcomes in Patient Subgroups Classified by PD-L1 Expression

Since PD-L1 expression is a major factor that influences therapy choices, we investigated the model's ability to predict survival outcomes when considering PD-L1 classification. In our cohort, PD-L1-high patients (≥50%) displayed the best outcome, with up to two-fold difference in median OS and PFS in comparison to PD-L1-low (1-49%) and PD-L1-negative (<1%) patients (FIG. 28). Most PD-L1-high patients (65.3%) were treated with ICI monotherapy, in line with the current guidelines.

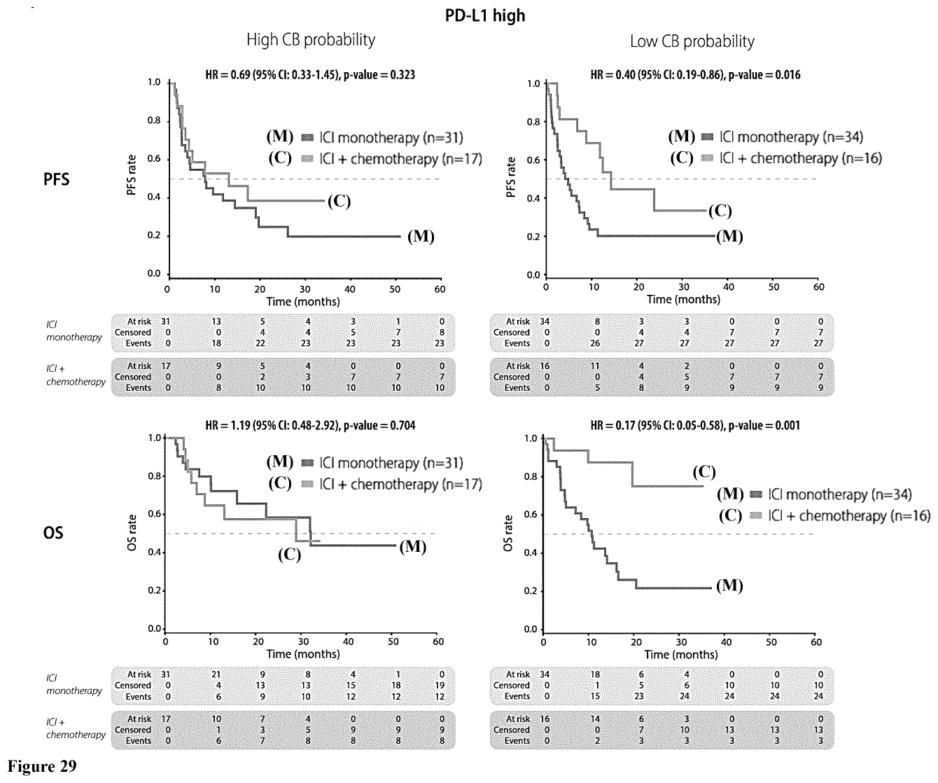

Among the PD-L1-high patients, it is possible to differentiate between patients who would benefit from ICI monotherapy and those who would fare better with combination ICI-chemotherapy. To explore this, the ability of the 12-month RAP model to forecast survival outcomes in PD-L1-high patients receiving ICI monotherapy or combination of ICI-chemotherapy was tested. Patients were classified into high or low CB probability groups using the cohort median CB probability as the threshold, and OS and PFS curves were plotted per group. In the high CB probability group, patients receiving ICI monotherapy or combination therapy fared similarly well (FIG. 29, left panel). Median OS was 32.13 vs 28.95 months and median PFS was 7.85 vs 13.08 months (monotherapy vs combination therapy). This suggests that such patients are suitable candidates for monotherapy and may be spared the more toxic ICI-chemotherapy combination. In contrast, in the low CB probability group, OS and PFS were significantly longer in patients receiving ICI-chemotherapy in comparison to ICI monotherapy (FIG. 29, right panel). Median OS was not reached versus 10.71 months (combination vs monotherapy; HR=0.17; p=0.001) and median PFS was 14.29 vs. 4.14 months (combination vs monotherapy; HR=0.40; p=0.016). This suggests that PD-L1-high patients with low CB probability should rather be treated with combination ICI-chemotherapy despite high PD-L1 levels.

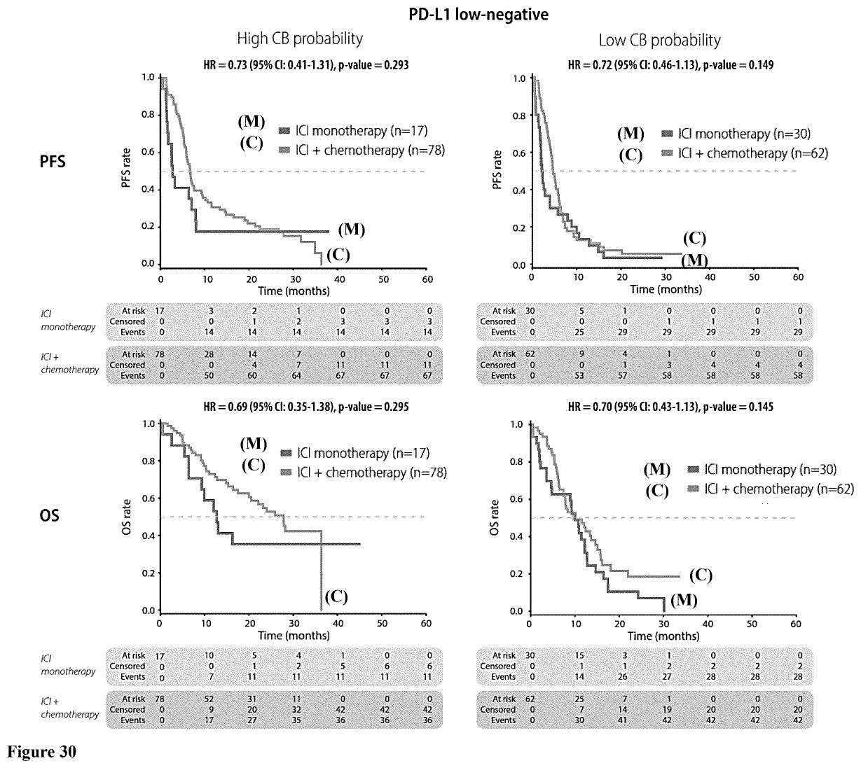

Also, it was asked whether the model could provide insights for managing patients with PD-L1<50%. To this end, the ability of the RAP model to forecast survival outcomes in a mixed group of PD-L1-low and PD-L1-negative patients receiving ICI monotherapy or combination ICI-chemotherapy was tested (overall, 47 PD-L1-low and negative patients received ICI monotherapy, while 87% of them were treated with ICI as an advanced line of treatment). In this analysis, patients in the high CB probability group displayed an OS benefit when treated with ICI-chemotherapy combination in comparison to patients receiving monotherapy, although statistical significance was not reached (FIG. 30, left panel). Median OS was 27.83 months for ICI-chemotherapy vs 12.72 months for ICI monotherapy. Notably, the median OS in the ICI-chemotherapy subset was comparable to that of PD-L1-high patients overall (median OS 28.96; FIG. 28). This result is in line with current guidelines recommending ICI-chemotherapy rather than ICI monotherapy for patients with PD-L1<50%. However, patients in the low CB probability group displayed similarly poor outcomes when treated with either of the two treatment modalities, with a median OS of 10.02 and 9.69 months for monotherapy and ICI-chemotherapy, respectively (FIG. 30, right panel). This suggests that treatment types other than the typically used ICI-chemotherapy combinations including platinum-based chemotherapy, Ist line clinical trials and novel combination treatments could be considered for this patient subgroup. Similar trends were observed when performing such comparisons in subgroups comprised only of PD-L1-low or PD-L1-negative patients. While results presented here were obtained using the RAP model based on the 12-month time point, similar results were obtained with the 3- and 6-month time point models (data not shown).

These collective findings demonstrate the potential clinical utility of the model for optimizing treatment choices. When used in conjunction with PD-L1 testing, the model may help to determine whether a patient should receive ICI alone, an ICI-chemotherapy combination or an alternative to typically used therapies.

### Example 8: Further Confirmation of the RAP (PROphet) Model Forecasts

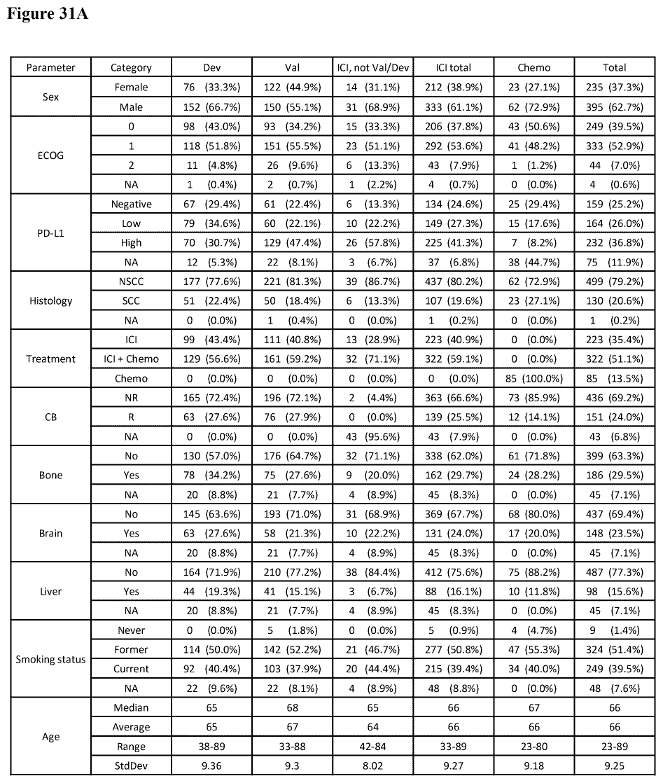

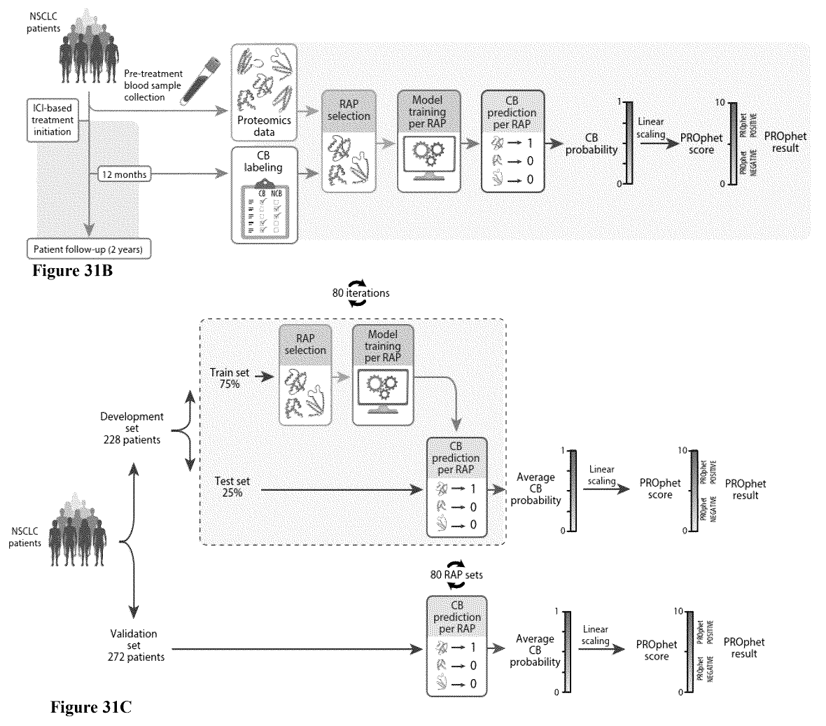

Blood plasma samples and clinical data were collected from 610 advanced stage NSCLC patients treated with ICI as monotherapy or ICI in combination with chemotherapy within the framework of the PROPHETIC clinical study (NCT04056247). A separate cohort of 85 patients treated with chemotherapy alone was used for certain comparisons. Samples analyzed in this study were analyzed for proteomic profiling of about 7000 proteins. Of the 610 enrolled patients, 65 were excluded due to technical or clinical reasons, resulting in 545 patients in the analyzed cohort (FIG. 31A). Clinical benefit (CB) was assessed 12 months after commencement of treatment. Patients displaying progression-free survival (PFS) for at least 12 months after starting treatment were classified as CB patients. All other patients were classified as ‘no clinical benefit’ (NCB) patients.

Patient clinical parameters are presented in FIG. 31B. Focusing on the ICI-based therapy cohort, the median age was 66 years (range of 33-89) with a predominance of male patients (61%). Most of the patients (80%) had non-squamous cell carcinoma, and ECOG performance status of 0-1 (91%). In terms of clinically important metastatic sites, 30%, 16% and 24% of the patients had bone, liver or brain metastasis, respectively. Overall, 25%, 27% and 41% of the patients had PD-L1 levels of <1%, 1-49% and ≥50%, respectively. Patients were treated either with ICI-chemotherapy combinations (59%) or ICI monotherapy (41%). Most of the patients were either former smokers (51%) or current smokers (39%). Overall, 25% of the patients achieved CB at 12 months.

A proteomic-based model development and evaluation was performed on patients receiving ICI-based therapy who had clinical benefit evaluation. The model was developed on a development set (n=228) and tested in a blind manner on an independent validation set (n=272; FIG. 31C). A set of 388 proteins (Table 4) that displayed differential plasma level distributions between CB and NCB populations was identified using Kolmogorov-Smirnov statistical test in 80 iterations of randomly selected training and test sets (FIG. 31C). These proteins, termed resistance associated proteins (RAPs), serve as potential indicators of CB based on XGBoost algorithm; the sum of 388 predictions in a given patient, called a PROphet score (total response score), reflects the patient's likelihood of benefiting from treatment.

As PD-L1-based tests are currently used for treatment guidance in NSCLC patients, the predictive performance of the PD-L1 biomarker on the validation set was evaluated. In this study, cancers with PD-L1≥50% displayed non-significant overall survival (OS) benefit compared to PD-L1<50% cancers (p-value=0.0655; hazard ratio, HR, between PD-L1≥50% and PD-L1<50% of 0.74, confidence interval, CI, of 0.53-1.02; FIG. 32A). Additionally, the PD-L1-based predictive model displayed a poor correlation between predicted clinical benefit probability and observed benefit rate (R²=0.35; FIG. 32B), demonstrating limited predictive capabilities of the PD-L1 biomarker.

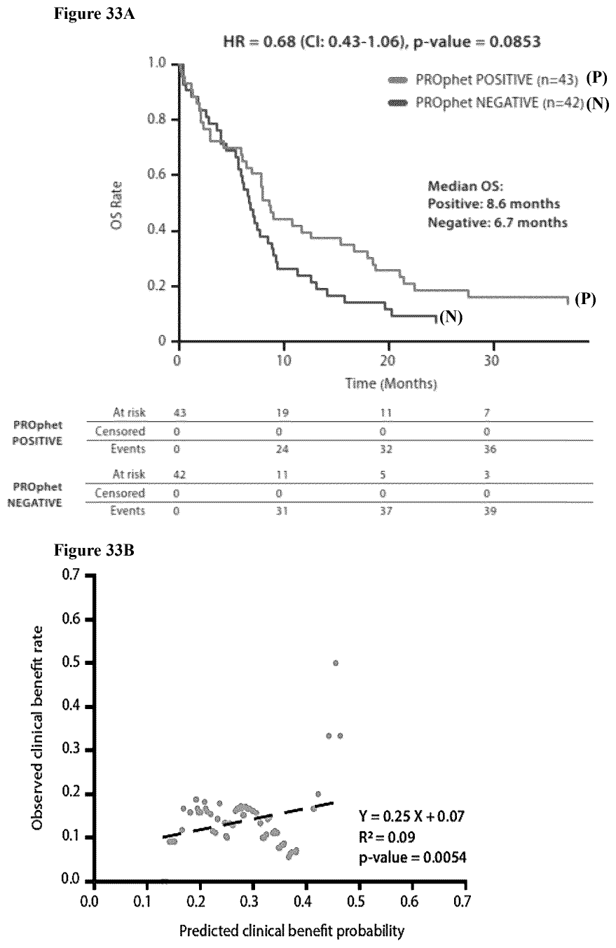

Using the proteomic and clinical data from the patients receiving ICI-based treatment, a model outputting CB probability (a continuous metric) was created. Patients with a predicted CB probability equal to or above versus below the median in the development set were classified into positive or negative groups, respectively. This proteomics-based model was termed PROphet (FIG. 31C-31D). This model displayed superior predictive performance in comparison to PD-L1, with a hazard ratio (HR) of 0.51 between the positive and -negative groups (CL=0.37-0.70; p-value<0.001; FIG. 32C) and a median OS of 25.9 and 10.8 months, for positive and -negative groups, respectively. Furthermore, the model demonstrated a high goodness of fit between the predicted CB probability and observed CB rate (R²=0.97; FIG. 32D), altogether demonstrating strong predictive performance. When examining the model capabilities on a retrospective cohort of treatment-naïve patients receiving chemotherapy alone, PROphet subgroups did not display a significant difference in OS (HR=0.68; CI=0.43-1.06; p-value=0.0853) (FIG. 33A). The correlation between the predicted CB probability and the observed CB rate was poor (R²=0.09, FIG. 33B). Altogether, this implies that the PROphet test is predictive for ICI-based therapy rather than for chemotherapy.

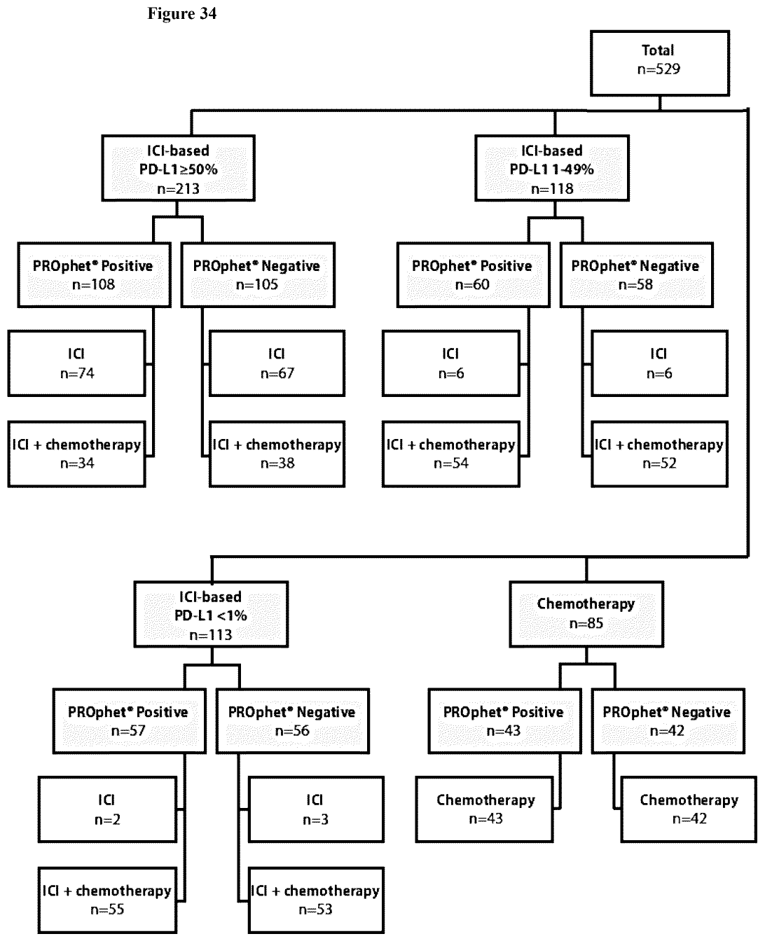

Next, the clinical utility of combining the model result with PD-L1 expression levels (patient stratification is indicated in FIG. 34) was evaluated. The subgroup with PD-L1≥50% patients with a positive result fared similarly well in terms of OS and PFS when receiving ICI monotherapy or combination therapy (OS HR=0.77; CI=0.42-1.43; p-value=0.4096;

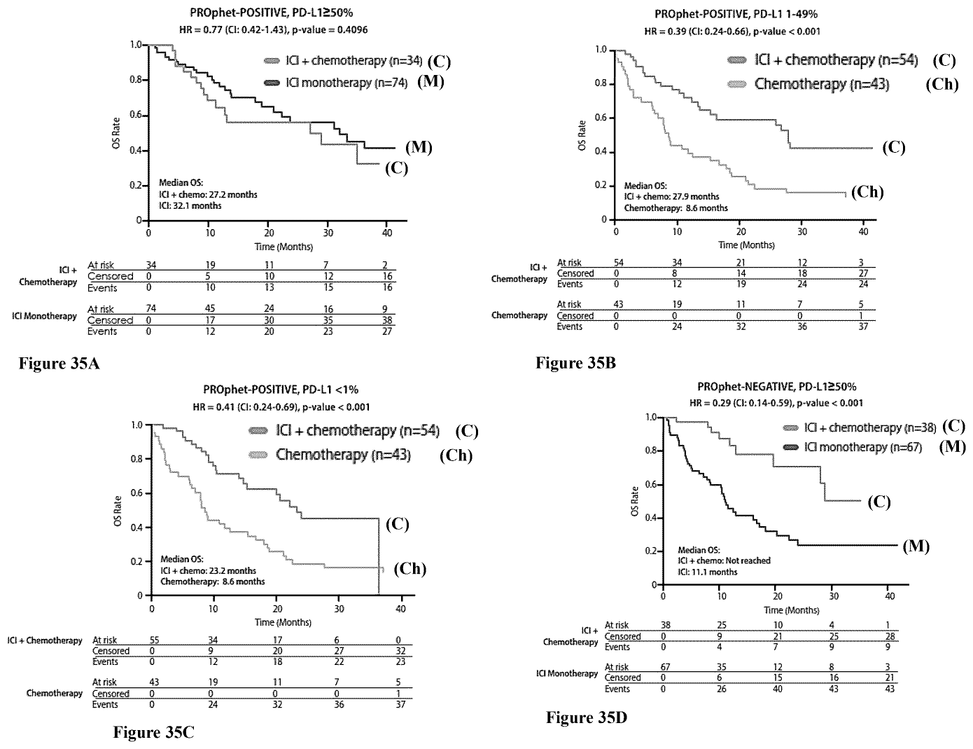

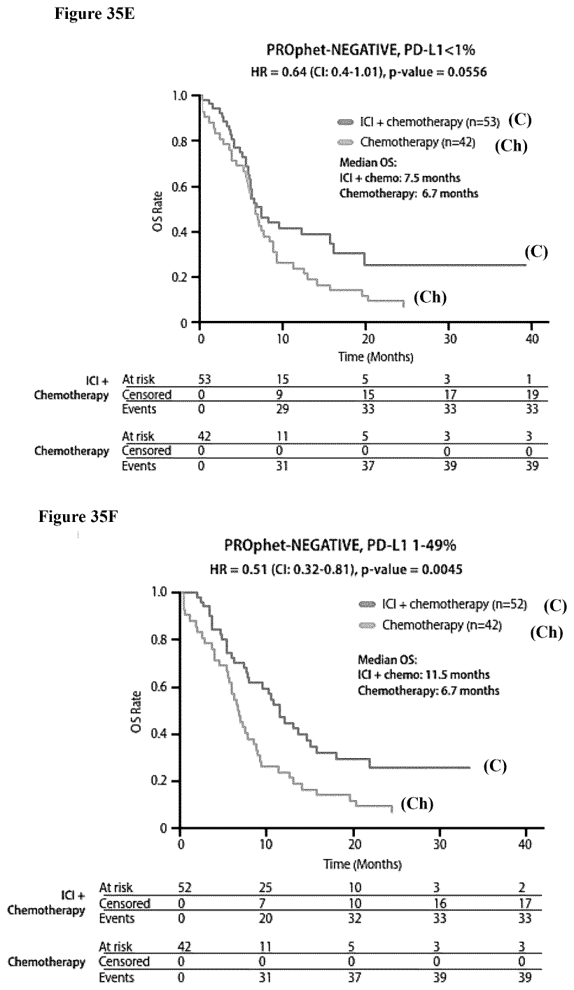

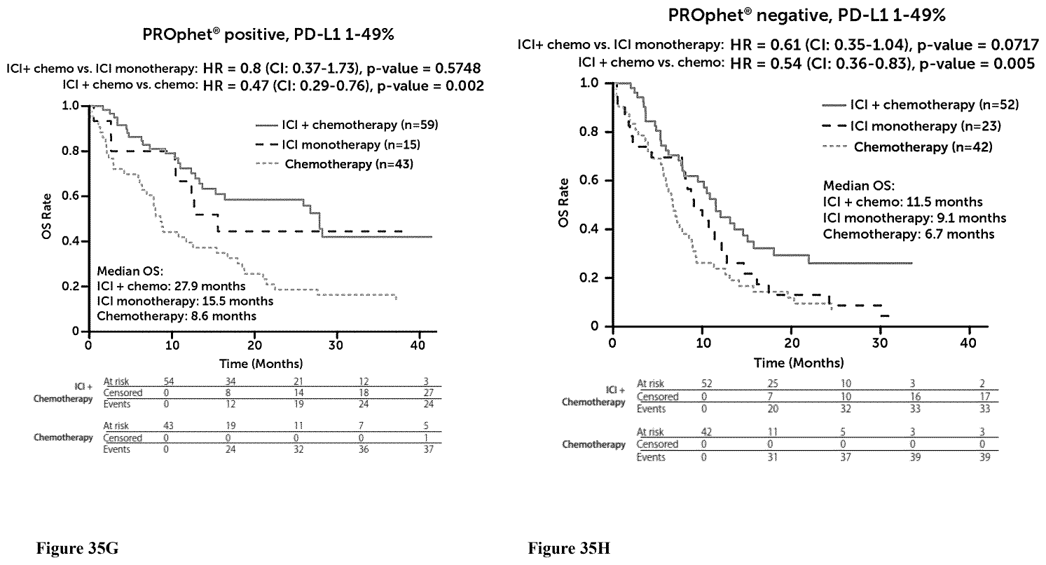

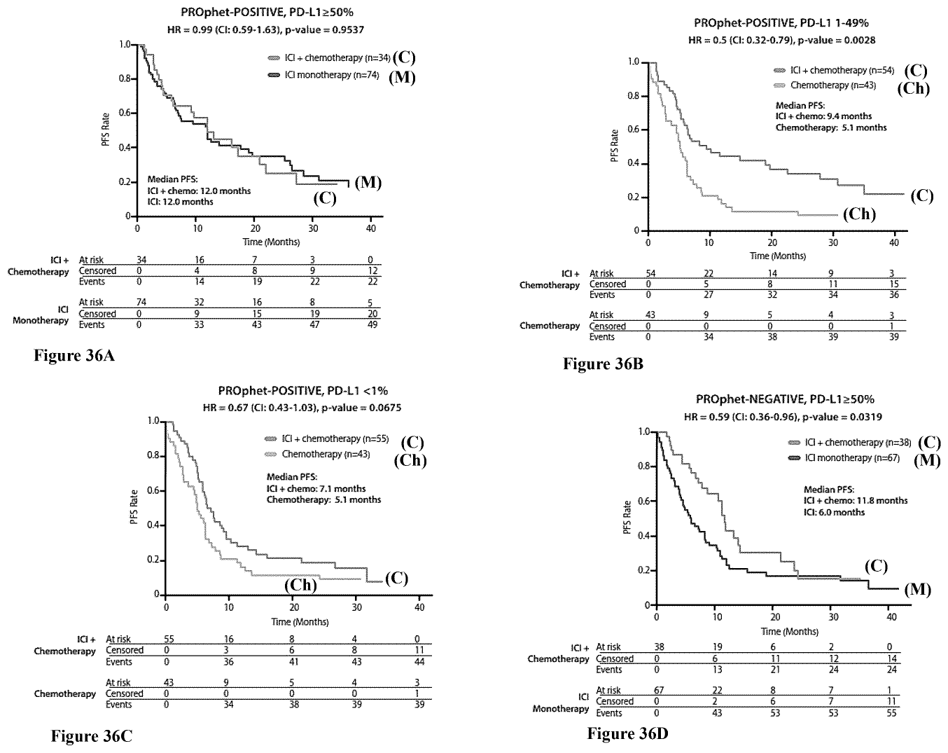

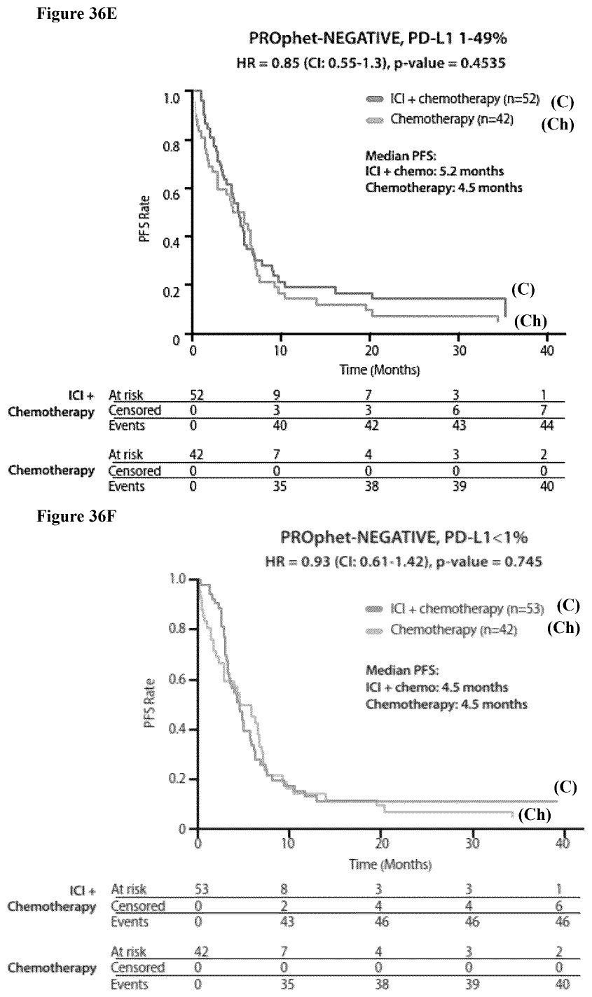

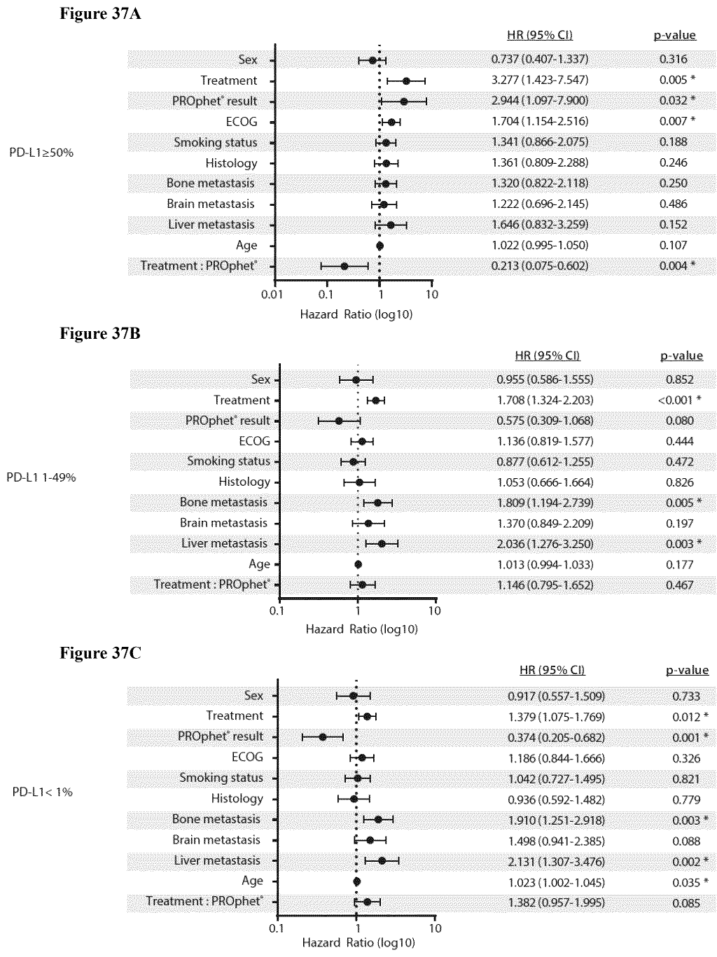

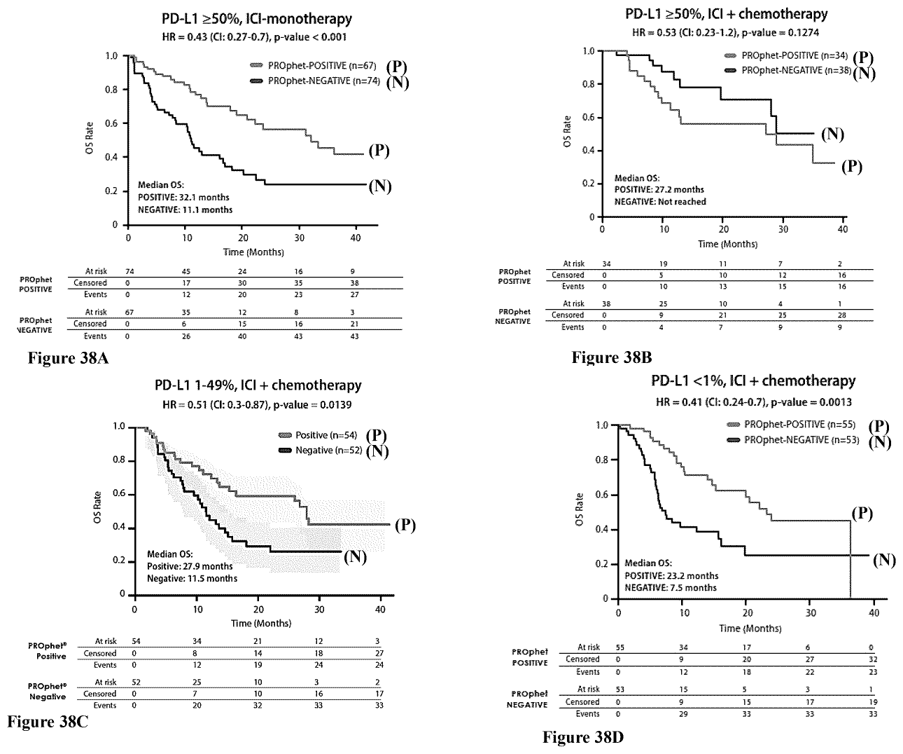

FIG. 35A and FIG. 36A). This implies that such patients are suitable candidates for monotherapy and may be spared the more toxic ICI-chemotherapy combination. In contrast, in PD-L1≥50% patients with negative result, both OS and PFS were significantly longer when receiving ICI-chemotherapy in comparison to ICI monotherapy (FIG. 35D and FIG. 36D), with a median OS that was not reached versus 11.10 months in the combination therapy and monotherapy groups, respectively (HR=0.29; CI=0.14-0.59; p-value<0.001). Multivariate Cox proportional hazard regression analysis identified a significant interaction between the model result and treatment regimen (FIG. 37, ECOG Performance Status Scale was also significant), indicating that treatment effect is dependent on the model result. This suggests that PD-L1≥50% patients with a negative result should consider combination of ICI-chemotherapy despite high PD-L1 levels, in contrast to the patients with the positive result. This coincides with a comparison between the negative and positive subgroups with PD-L1≥50%; where a significant difference between the two groups was observed for ICI-monotherapy but not for ICI-chemotherapy combination (FIG. 38A-38B).

Next, the subgroup of PD-L1<50% was analyzed. The subgroup of PD-L1<50% patients with a positive result displayed a significant benefit in OS for ICI-chemotherapy combination over chemotherapy alone (FIG. 35B-35C) with HR of 0.39 and 0.41 for PD-L1 1-49% and <1% patients, respectively, and median OS of 27.9 and 23.2 months for PD-L1 1-49% and <1% patients receiving ICI-chemotherapy, respectively, versus 8.6 months for chemotherapy. PFS was beneficial for ICI-chemotherapy only in patients with PD-L1 1-49% (FIG. 36B), while no significant difference was observed in PFS for PD-L1<1% (HR=0.67, CI=0.43-1.03; p-value=0.0675 (FIG. 36C). Notably, in the PD-L1<50% group, the ICI-monotherapy was used in the comparison only for the PD-L1 1-49% group as it is not standard of care for these patients (FIG. 35G-35H). In addition, patients receiving chemotherapy were not stratified based on PD-L1 expression level, as the value was not available for many of the patients treated with chemotherapy alone. Overall, our findings suggest that PD-L1<50% patients with a positive result benefit from guidelines-based treatment (i.e., ICI-chemotherapy combination).

When examining patient subgroup with PD-L1 1-49% and a negative result, a significant difference between ICI-chemotherapy and chemotherapy alone was observed, with HR of 0.51 and median OS of 11.5 and 6.7 months in combination therapy versus chemotherapy, respectively (FIG. 35F), while no significant difference in PFS was observed between the two arms (FIG. 36E). In a multivariate analysis there was no interaction between treatment and PROphet result, indicating that on the subgroup of PD-L1 1-49% patients there is no effect of the result on the treatment, as both negative and positive patients benefit from combination therapy. As our results displayed only a moderate benefit from ICI-chemotherapy treatment for PD-L1 1-49% patients with a PROphet negative result, these patients may want to consider other approved therapies or first-line clinical trials, as also recommended by NCCN guidelines.

Conversely, negative patients with PD-L1<1% displayed similarly poor outcomes for both treatment modalities, with median OS of 7.5 and 6.7 months for combination therapy and chemotherapy, respectively (FIG. 35E), and median PFS of 4.5 months for both treatment modalities (FIG. 36F). These findings suggest that such patients are not likely to benefit from ICI-based combination therapy over chemotherapy and may choose to consider other approved therapies or first-line clinical trials. In accordance, PD-L1<1% patients displayed a significant difference between the negative and positive subgroups (FIG. 38D). The guidelines for patients with PD-L1≥50% support the usage of either ICI-monotherapy or ICI combined with chemotherapy, while there is no clear guidance as to which treatment modality will be more beneficial for these patients. The model of invention can successfully differentiate between patients who would benefit from the combination therapy and those who can suffice with ICI-monotherapy and may avoid chemotherapy-related toxicity. The test can improve overall survival rates by guiding PD-L1≥50% patients with negative response scores to ICI-chemotherapy treatment modality.

The guidelines for patients with PD-L1<50% recommend administering ICI-chemotherapy in combination. Patients with PROphet positive response scores and either PD-L1 1-49% or PD-L1<1% expression levels displayed prolonged OS when receiving ICI combined with chemotherapy; therefore, the test successfully identifies the patients who can benefit from standard of care. However, patients with negative response scores displayed differential results for PD-L1 1-49% and PD-L1<1% expression levels; while patients with PD-L1 1-49% displayed significant benefit for the combination therapy, PD-L1<1% patients did not show such significant difference.

The PD-L1 biomarker is currently used to guide treatment selection, however, is not fully trusted, as previously described. The described model of invention provides a proteomic analysis of a pre-treatment plasma sample in combination with PD-L1 test for stratification of the patients into subgroups that provide additional resolution to consider when selecting treatment regimen, thus providing a novel tool for therapeutic decision-making and clinical benefit prediction in NSCLC patients receiving ICI-based therapy, thus addressing an unmet need.

TABLE 4
Resistance associated proteins (RAPs) that are in the basis of the PROphet model

| Gene | UniProt | Gene | UniProt | Gene | UniProt | Gene | UniProt |
|---|---|---|---|---|---|---|---|
| name | ID | name | ID | name | ID | name | ID |
| KCNAB2 | Q13303 | DLD | P09622 | NTAN1 | Q96AB6 | HRG | P04196 |
| IL12B; | P29460; | EPHB4 | P54760 | STARD7 | Q9NQZ5 | SCGB2A1 | O75556 |
| IL23A | Q9NPF7 | | | | | | |
| MCL1 | Q07820 | PRSS27 | Q9BQR3 | APOL2 | Q9BQE5 | SIRT2 | Q8IXJ6 |
| KIR2DS2 | P43631 | MUC16 | Q8WXI7 | FLT4 | P35916 | TNFAIP6 | P98066 |
| AGA | P20933 | CFHR2 | P36980 | RCSD1 | Q6JBY9 | CD300C | Q08708 |
| RPN1 | P04843 | HTRA1 | Q92743 | INIP | Q9NRY2 | GPNMB | Q14956 |
| LAT | O43561 | KRT19 | P08727 | VMAC | Q2NL98 | KRT18 | P05783 |
| MFAP2 | P55001 | RBP4 | P02753 | XPNPEP3 | Q9NQH7 | TNFSF14 | O43557 |
| PUF60 | Q9UHX1 | SMOC2 | Q9H3U7 | IFNE | Q86WN2 | LEPR | P48357 |
| MPZ | P25189 | BTD | P43251 | NELFA | Q9H3P2 | PRKCG | P05129 |
| ACE | P12821 | TXLNA | P40222 | KDM8 | Q8N371 | FGL1 | Q08830 |
| RNF122 | Q9H9V4 | MZB1 | Q8WU39 | NCBP1 | Q09161 | PGLYRP2 | Q96PD5 |
| TXNDC5 | Q8NBS9 | FADD | Q13158 | USF2 | Q15853 | NPFF | O15130 |
| CDH15 | P55291 | GSN | P06396 | LRRC75A | Q8NAA5 | MFAP4 | P55083 |
| FGFBP3 | Q8TAT2 | CDH17 | Q12864 | APCS | P02743 | TMX3 | Q96JJ7 |
| COL11A2 | P13942 | LECT2 | O14960 | PLCD1 | P51178 | PRKCSH | P14314 |
| INPP5E | Q9NRR6 | ADAMTSL1 | Q8N6G6 | ESPN | B1AK53 | DEFB112 | Q30KQ8 |
| ADH7 | P40394 | RNASET2 | O00584 | RFX5 | P48382 | SEMA4D | Q92854 |
| MVK | Q03426 | SEMA4A | Q9H3S1 | RPS6KB2 | Q9UBS0 | ACP6 | Q9NPH0 |
| RNF146 | Q9NTX7 | DDOST | P39656 | NOMO2 | Q5JPE7 | AFP | P02771 |
| SOCS3 | O14543 | BDH2 | Q9BUT1 | TCEAL2 | Q9H3H9 | NGF | P01138 |
| RBFOX2 | O43251 | SNRPB2 | P08579 | CES3 | Q6UWW8 | FTH1; FTL | P02794; |
| | | | | | | | P02792 |
| ARFGAP1 | Q8N6T3 | GOLM1 | Q8NBJ4 | DYRK1A | Q13627 | DMKN | Q6EOU4 |
| SRSF6 | Q13247 | RAB3A | P20336 | CYP2C19 | P33261 | EPHA10 | Q5JZY3 |
| RBM23 | Q86U06 | CD46 | P15529 | CFI | P05156 | CHRDL2 | Q6WN34 |
| DDR1 | Q08345 | SEPTIN6 | Q14141 | IGFBP3 | P17936 | TP53 | P04637 |
| APOF | Q13790 | WWOX | Q9NZC7 | IL6 | P05231 | AOC1 | P19801 |
| TRA2B | P62995 | WDR5 | P61964 | LEP | P41159 | IFNA8 | P32881 |
| MCTS1 | Q9ULC4 | HPCAL1 | P37235 | CRTC3 | Q6UUV7 | CSH1; | P0DML2; |
| | | | | | | CSH2 | P0DML3 |
| TBCA | O75347 | ALDH5A1 | P51649 | VEGFA | P15692 | TNC | P24821 |
| RGS7 | P49802 | VAT1 | Q99536 | IL1RAP | Q9NPH3 | PLTP | P55058 |
| PTPN9 | P43378 | SARS1 | P49591 | HGF | P14210 | CCN1 | O00622 |
| CSNK1G2 | P78368 | AFM | P43652 | PLA2G2A | P14555 | CLSTN3 | Q9BQT9 |
| ILF3 | Q12906 | CDA | P32320 | CCL25 | O15444 | OIT3 | Q8WWZ8 |
| TPPP2 | P59282 | ITLN1 | Q8WWA0 | SERPINA7 | P05543 | GGT2 | P36268 |
| ARHGEF2 | Q92974 | LRIG1 | Q96JA1 | POR | P16435 | FMOD | Q06828 |
| SRSF7 | Q16629 | GREM1 | O60565 | CCN3 | P48745 | C5orf38 | Q86SI9 |
| EWSR1 | Q01844 | PTGR2 | Q8N8N7 | HPX | P02790 | VWA1 | Q6PCB0 |
| FSTL1 | Q12841 | UBE2L6 | O14933 | IGFBP1 | P08833 | INHBC | P55103 |
| SPP1 | P10451 | CLTA | P09496 | MMP3 | P08254 | ADGRF5 | Q8IZF2 |
| FLRT2 | O43155 | GSR | P00390 | FGA; | P02671; | C1QL2 | Q7Z5L3 |
| | | | | FGB; FGG | P02675; | | |
| | | | | | P02679 | | |
| FLRT3 | Q9NZU0 | PDCD6 | O75340 | BCAM | P50895 | PCYOX1 | Q9UHG3 |
| VTN | P04004 | SNCG | O76070 | SPINT1 | O43278 | AOC2 | O75106 |
| ATP1B1 | P05026 | CRH | P06850 | HAT1 | O14929 | CFHR4 | Q92496 |
| WFIKKN2 | Q8TEU8 | RGS21 | Q2M5E4 | GHR | P10912 | LRRC15 | Q8TF66 |
| NRAC | Q8N912 | UBE2R2 | Q712K3 | CFP | P27918 | POSTN | Q15063 |
| PKD2 | Q13563 | BASP1 | P80723 | CNTN1 | Q12860 | UBE2J1 | Q9Y385 |
| HSPA9 | P38646 | GBP5 | Q96PP8 | SERPINF2 | P08697 | GFRAL | Q6UXV0 |
| EMC4 | Q5J8M3 | LMNB2 | Q03252 | IL19 | Q9UHD0 | IGF2 | P01344 |
| ASAP2 | O43150 | POP7 | O75817 | MB | P02144 | LILRB5 | O75023 |
| NAP1L2 | Q9ULW6 | RAET1L | Q5VY80 | C9 | P02748 | LILRA6 | Q6PI73 |
| HTR7 | P34969 | SEMA5B | Q9P283 | IGHM | P01871 | APOA2 | P02652 |
| DCUN1D3 | Q8IWE4 | CNTN3 | Q9P232 | LBP | P18428 | VWA2 | Q5GFL6 |
| RBL2 | Q08999 | UBL3 | O95164 | NAAA | Q02083 | DEPP1 | Q9NTK1 |
| MAD1L1 | Q9Y6D9 | MMACHC | Q9Y4U1 | HAPLN1 | P10915 | C1QTNF3 | Q9BXJ4 |
| GRB14 | Q14449 | GTF2B | Q00403 | IDS | P22304 | SERPINA9 | Q86WD7 |
| RBBP5 | Q15291 | GCHFR | P30047 | NID1 | P14543 | CFHR5 | Q9BXR6 |
| NAB2 | Q15742 | LRATD2 | Q96KN1 | ACAN | P16112 | DLG3 | Q92796 |
| CSF1R | P07333 | SGK1 | O00141 | TGFBI | Q15582 | GLTPD2 | A6NH11 |
| CCN4 | O95388 | TSEN15 | Q8WW01 | DLL4 | Q9NR61 | HBQ1 | P09105 |
| GPD1 | P21695 | SAR1B | Q9Y6B6 | FCGR3B | O75015 | ENTPD1 | P49961 |
| KLK3 | P07288 | CDK5RAP3 | Q96JB5 | ACY1 | Q03154 | AGGF1 | Q8N302 |
| CXCL13 | O43927 | HAUS1 | Q96CS2 | IBSP | P21815 | NRG2 | O14511 |
| GZMA | P12544 | NKIRAS1 | Q9NYSO | SERPINA4 | P29622 | SPON2 | Q9BUD6 |
| C9 | P02748 | PHOSPHO2 | Q8TCD6 | POSTN | Q15063 | FAM241B | Q96D05 |
| IL12B | P29460 | PCDH17 | O14917 | SELE | P16581 | JAML | Q86YT9 |
| RAP1GAP | P47736 | TRIM5 | Q9C035 | B2M | P61769 | BCHE | P06276 |
| IGFBP1 | P08833 | ALDH7A1 | P49419 | HAMP | P81172 | GPNMB | Q14956 |
| DHX58 | Q96C10 | TXNL4A | P83876 | SERPINA1 | P01009 | APOD | P05090 |
| COPS2 | P61201 | CEP20 | Q96NB1 | AHSG | P02765 | DLL1 | O00548 |
| IL1RAP | Q9NPH3 | PDE1B | Q01064 | CKB; | P12277; | PEAR1 | Q5VY43 |
| | | | | CKM | P06732 | | |
| CCL25 | O15444 | ITGA4; | P13612; | PROC | P04070 | RSPO4 | Q2I0M5 |
| | | ITGB1 | P05556 | | | | |
| HPX | P02790 | LRFN3 | Q9BTN0 | PROC | P04070 | LEP | P41159 |
| ADM | P35318 | ADGRB1 | O14514 | ANGPTL4 | Q9BY76 | ARL8B | Q9NVJ2 |
| CD93 | Q9NPY3 | SGSH | P51688 | MBD4 | O95243 | PCDH10 | Q9P2E7 |
| ISG15 | P05161 | MGAT5 | Q09328 | PSMD7 | P51665 | MFAP3L | O75121 |
| MYL6B | P14649 | B3GAT1 | Q9P2W7 | IGHE | P01854 | CD14 | P08571 |
| HSPA1A | P0DMV8 | MGAT5 | Q09328 | CXCL10 | P02778 | COL15A1 | P39059 |
| MBD1 | Q9UIS9 | FBLN7 | Q53RD9 | KLKB1 | P03952 | PCDH10 | Q9P2E7 |
| TRAPPC3 | O43617 | APBB1IP | Q7Z5R6 | CFH | P08603 | HAVCR1 | Q96D42 |
| AKT2 | P31751 | PON2 | Q15165 | PFDN5 | Q99471 | ARHGEF10 | O15013 |
| CRLF1 | O75462 | PPP2R5D | Q14738 | RBM39 | Q14498 | MAN1A2 | O60476 |
| FTL | P02792 | RBFOX1 | Q9NWB1 | DCTPP1 | Q9H773 | CRYZL1 | O95825 |
| RBBP4 | Q09028 | TIMP1 | P01033 | PRSS22 | Q9GZN4 | TFPI2 | P48307 |
| BMPER | Q8N8U9 | GEMIN7 | Q9H840 | KYNU | Q16719 | PLXDC1 | Q8IUK5 |
| SERPINB5 | P36952 | CSNK1A1L | Q8N752 | IL6 | P05231 | ACP2 | P11117 |
| PMP2 | P02689 | PHF11 | Q9UIL8 | AFM | P43652 | BTD | P43251 |
| OTC | P00480 | BTN2A2 | Q8WVV5 | SERPINA6 | P08185 | MFAP2 | P55001 |
| OTOR | Q9NRC9 | SKP2 | Q13309 | ITIH4 | Q14624 | ITIH2 | P19823 |
| AOC1 | P19801 | SPATA46 | Q5T0L3 | SFN | P31947 | EFCAB14 | O75071 |
| FGFBP1 | Q14512 | LIN7A | O14910 | CCL7 | P80098 | PLA1A | Q53H76 |
| ATRN | O75882 | BORCS5 | Q969J3 | LYZ | P61626 | GZMK | P49863 |
| NAGLU | P54802 | ARRDC5 | A6NEK1 | MMP13 | P45452 | YBX1 | P67809 |
| SAA1 | P0DJI8 | PCYT1A | P49585 | STC1 | P52823 | IDO1 | P14902 |
| SAA4 | P35542 | PHYH | O14832 | CAPG | P40121 | NQO1 | P15559 |
| CLSTN1 | O94985 | ANKRD63 | C9JTQ0 | PI3 | P19957 | SPOCK3 | Q9BQ16 |
| GSS | P48637 | VCX | Q9H320 | GPC5 | P78333 | NXT1 | Q9UKK6 |

### Example 9: Evaluation of the Response Prediction Using the PROphet Model in Melanoma and SCLC Patients

It was hypothesized that immunotherapy response encompasses common mechanisms across cancer types. Therefore, the NSCLC response prediction classifier was applied to protein measurements from blood samples from subjects with various other cancers within the framework of the PROPHETIC clinical study (NCT04056247).

TO blood plasma samples and clinical data were collected from 68 non-resectable metastatic melanoma patients treated with anti-PD1 alone or in combination with anti-CTLA-4. The response prediction model performance was quantified based on ROC AUC of CB prediction at 1 year. Specifically, the goodness of fit between predicted response probability and observed response probability was evaluated based on 1-year CB using R² distance from best fit line. Hazard-ratio (HR) between positive and negative patients was also computed.

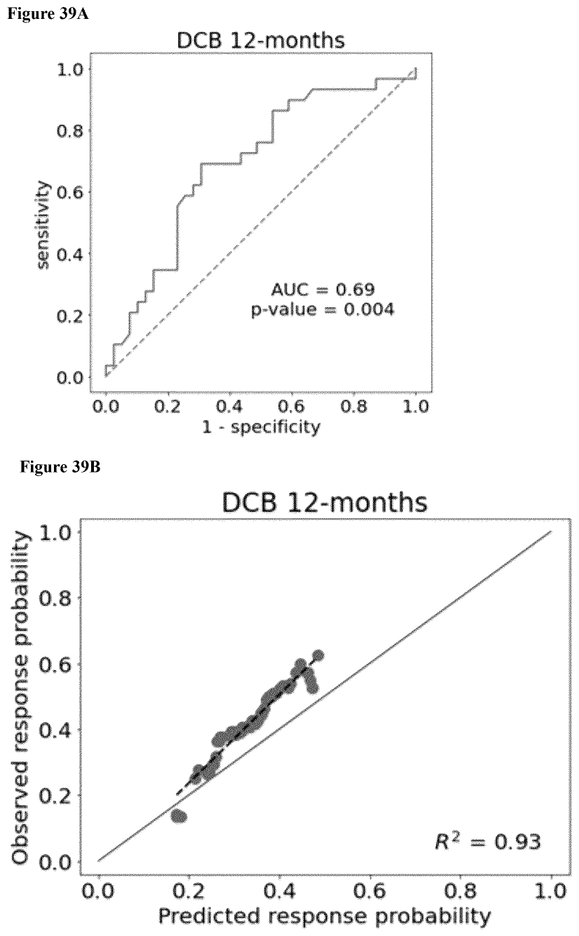

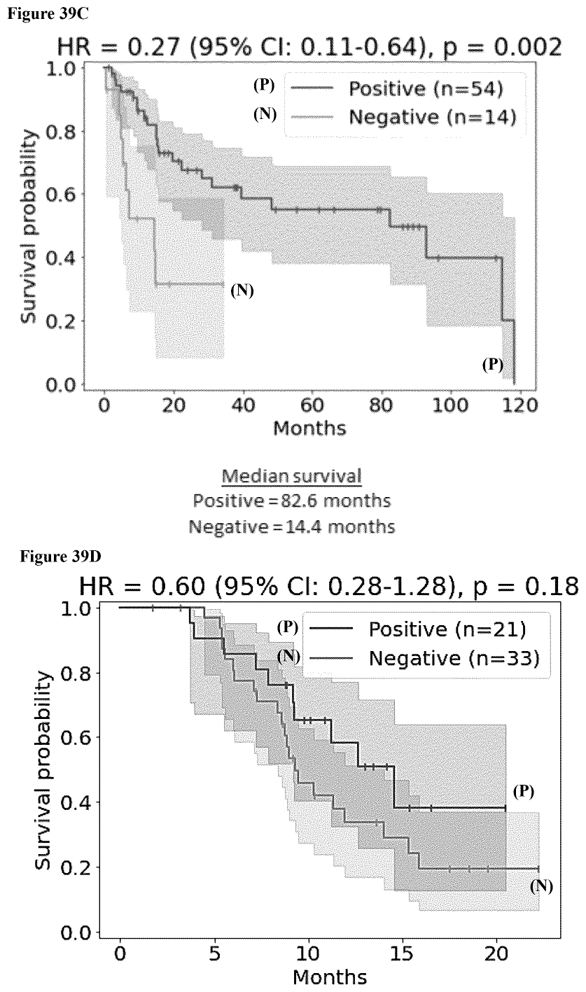

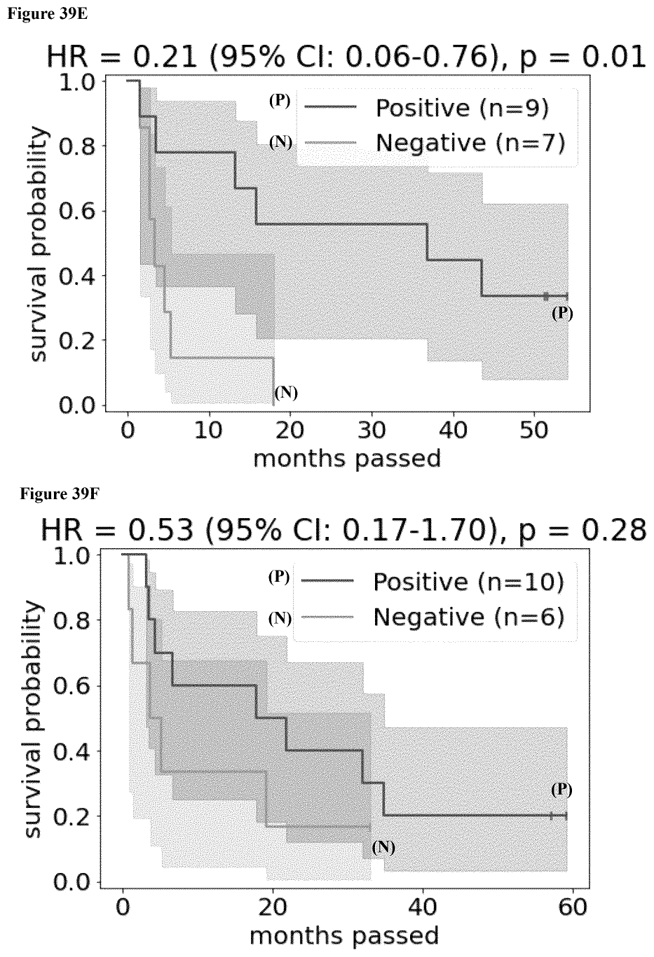

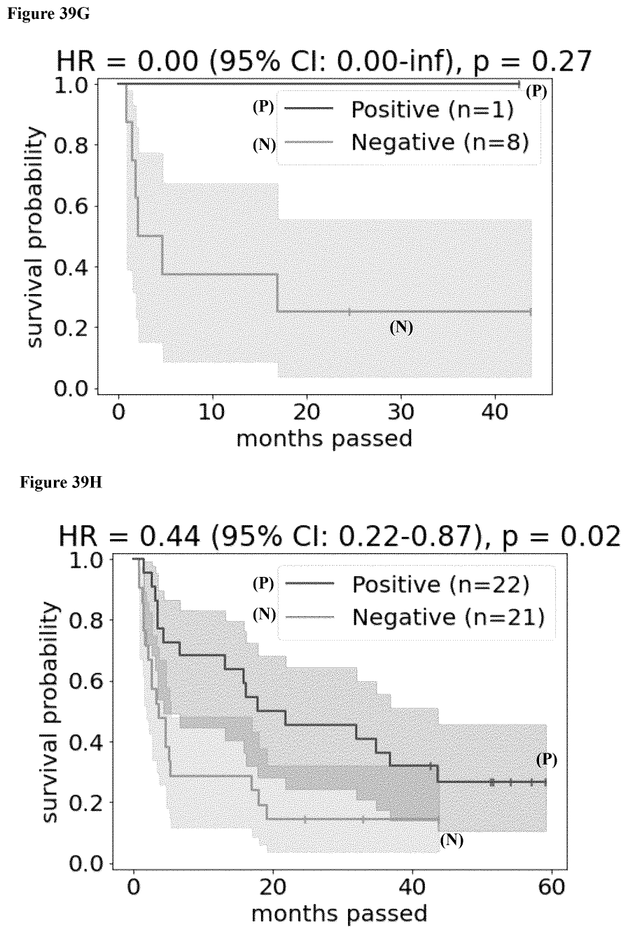

In order for the classifier to be considered predictive, the following criteria need to be met. First, the validation 1-year duration of CB ROC AUC needed to be above 0.60 with a p-value below 0.05. The threshold of 0.6 was selected to assure that the model response probability performs better than random and is relatively low. A more stringent threshold was not selected as the goodness of fit is a more important criterion. The second criterion was goodness of linear fit between predicted response probability and observed response probability. For 1-year duration of CB the fit should be above R\hat{ }2>0.85 relative to best-fit line. The slope should be higher than 0.9. Third, the predicted response probability for 1-year CB should span a range of at least 0.25 (i.e., if the higher response probability that was assigned to a patient in the validation set is 0.6, the lowest response probability should be 0.35 or lower). Finally, the hazard-ratio between the positive and negative patients should be below 0.8. As can be seen in FIGS. 39A-39C, the Model ROC AUC for 1-year durable clinical benefit was 0.69, p=0.004 (39A), the goodness of linear fit between the predicted and observed response probability based on the PROphet model was R^{2=0.93} (39B), the Kaplan Meier plots for PROphet positive and PROphet negative patients showed a Hazard ration of 0.27 (39C), and the predicted probability range was 0.27. Since all the acceptance criteria are met, it can be concluded that the classifier is also considered as predictive for response of melanoma patients treated with anti-PD-1 therapy.

A similar analysis was performed on TO plasma samples from a cohort of 54 small cell lung cancer (SCLC) patients. Patients with at least 7 months follow-up were included in the analysis, and response to treatment was defined as PFS of at least 7 months after treatment initiation. Patients were treated with combinations of ICI (48 atezolizumab, 6 durvalumab) and chemotherapy (carboplatin and etoposide). As can be seen from FIG. 39D, Kaplan Meier plots for PROphet positive and PROphet negative patients showed a Hazard ration of 0.60, p=0.18, showing that the classifier is also considered as predictive for response of SCLC patients to treatment with PD-(L)1 inhibitors.

### Example 10: Evaluation of the Response Prediction Using the PROphet Model in HPV-Related Malignancies

Patients suffering from HPV-related malignancies were also evaluated using the PROphet classifier (FIG. 39H). TO Scrum samples from a cohort of 43 patients suffering from HPV-related malignancies including anogenital (FIG. 39E), cervical (FIG. 39F), and head and neck cancer (FIG. 39G) and treated with anti-PDL1/TGFβ-Trap fusion protein were analyzed for their proteomic expression profile and for their survival probability using the PROphet classifier. As can be seen from the Kaplan Meier curves for PROphet positive and PROphet negative patients in FIGS. 39E-39H a Hazard ratio of the PROphet classifier was predictive for these cancers as well. This finding indicates that the resistance mechanisms being identified by the classifier are probably pan-cancer phenomenon and that the classifier is thus useful for all cancers.

### Example 11: Evaluation of the Response Prediction Using the PROphet Model in NSCLC Patients with Targetable Mutations

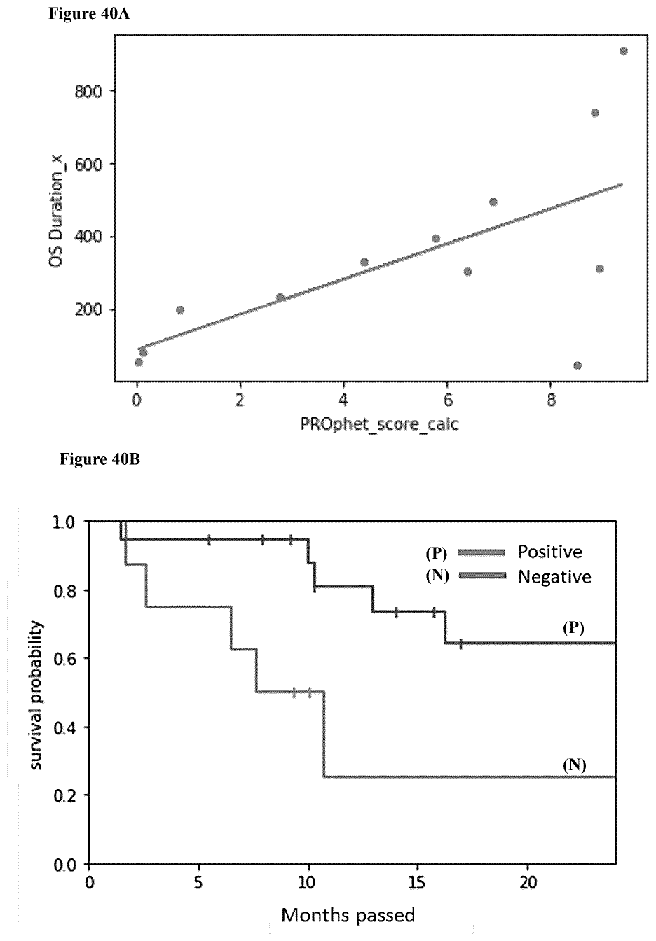

NSCLC patients having EGFR, ALK or ROS1 mutations usually do not respond well to immunotherapy and thus are first treated with tyrosine kinase inhibitors (TKIs). To date, there are no biomarkers for identification of NSCLC patients with EGFR, ALK or ROS1 mutations that are likely to benefit from treatment with PD-(L)1 inhibitors. A cohort of 35 advanced line NSCLC patients previously treated or not treated with TKIs prior to treatment with PD-(L)1 inhibitors was analyzed by the PROphet model. As can be seen in FIG. 40A, the goodness of linear fit between the PROphet score and the overall survival duration (days) was R²=0.41, p=0.0073. A Kaplan Meier curve for PROphet positive and PROphet negative patients showed a hazard ration of 0.36, p=0.07 (FIG. 40B). These results demonstrate the ability of the model to predict response to treatment in this specific sub-population, and also to differentiate between NSCLC patients with targetable mutations that may benefit from PD-(L)1 treatment (PROphet positive patients) and these that will not (PROphet negative patients).

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method of predicting response of a subject suffering from cancer to a therapy comprising an anti-PD-1/PD-L1 immunotherapy, the method comprising:
a, wherein said cancer is a PD-L1 high cancer and said therapy is a monotherapy comprising said anti-PD-1/PD-L1 immunotherapy, receiving factor expression levels for a plurality of factors
i. in a population of subjects suffering from cancer and known to respond to said monotherapy (responders);
ii. in a population of subjects suffering from cancer and known to not respond to said monotherapy (non-responders); and
iii. in said subject; or
wherein said cancer is a PD-L1 low or negative cancer and said therapy is a combination therapy comprising an anti-PD-1/PD-L1 immunotherapy and chemotherapy, receiving factor expression levels for a plurality of factors
i. in a population of subjects suffering from cancer and known to respond to said combination therapy (responders);
ii. in a population of subjects suffering from said cancer and known to not respond to said combined therapy (non-responders); and
iii. in said subject;
b. calculate for factors of said plurality of factors a resistance score, wherein said calculating comprises applying a machine learning algorithm trained on a training set comprising said received factor expression levels in responders and non-responders and the sex of each of said responders and non-responders to individual received factor expression levels from said subject and said subject's sex and wherein said machine learning algorithm outputs said resistance score; and
c. combine said calculated resistance scores to produce a total resistance score;
wherein a subject with a total resistance score beyond a predetermined threshold is predicted to not respond to said therapy and a subject with a total resistance score within said predetermined threshold is predicted to respond to said therapy;
thereby predicting response of a subject to a therapy.

2. The method of claim 1, wherein said total resistance score is converted to a total response score and wherein a total response score above a predetermined threshold indicates the subject is responsive to said therapy and a total response score below a predetermined threshold indicates the subject is not responsive to said therapy.

3. The method of claim 1, wherein said training set comprises received factor expression levels in subjects suffering from cancer and known to respond to a combination therapy comprising an anti-PD-1/PD-L1 immunotherapy and chemotherapy (combo-responders), received factor expression levels in subject suffering from cancer and known to not respond to said combination therapy (combo-non-responders), received factor expression levels in subjects suffering from cancer and known to respond to a therapy comprising an anti-PD-1/PD-L1 immunotherapy (mono-responders), received factor expression levels in subjects suffering from cancer and known to not respond to said therapy (mono-non-responders) and the sex of each of said combo-responders and combo-non-responders.

4. (canceled)

5. (canceled)

6. (canceled)

7. A method of predicting response of a subject suffering from cancer to an anti-PD-1/PD-L1 immunotherapy, the method comprising:
a. receiving factor expression levels for a plurality of factors
i. in a population of subjects suffering from cancer and known to respond to said immunotherapy (responders);
ii. in a population of subjects suffering from cancer and known to not respond to said immunotherapy (non-responders); and
iii. in said subject;
b. calculate for factors of said plurality of factors a resistance score, wherein said calculating comprises applying a machine learning algorithm trained on a training set comprising said received factor expression levels in responders and non-responders and the sex of each of said responders and non-responders, to individual received factor expression levels from said subject and said subject's sex and wherein said machine learning algorithm outputs said resistance score; and
c. combine said calculated resistance scores to produce a total resistance score;
wherein a subject with a total resistance score beyond a predetermined threshold is predicted to not respond to said anti-PD-1/PD-L1 immunotherapy;
thereby predicting response of a subject to an anti-PD-1/PD-L1 immunotherapy.

8. The method of claim 1, wherein said plurality of factors comprises at least two factors selected from the factors provided in Table 4, optionally wherein said plurality of factors consists of factors selected from Table 4.

9. (canceled)

10. The method of claim 1, wherein at least one of:
a. said responders and non-responders are determined based on progression free survival (PFS) at 1 year after initiation of said therapy or combination therapy;
b. said method comprises before (b) selecting a subset of said plurality of factors, wherein said subset comprises factors that best differentiate between said responders and non-responders, and wherein said calculating is for each factor of said subset and wherein said selecting comprises applying a statistical test to said received factor expression levels, optionally wherein said statistical test is a Kolmogorov-Smirnov test, said subset consists of at least 50 factors or both;
c. said factor expression level is from a time point before administration of an anti-PD-1/PD-L1 immunotherapy to said subject;
d. said combining is averaging;
e. said cancer is selected from hepato-biliary cancer, cervical cancer, urogenital cancer, anogenital cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer, renal cancer, skin cancer, head and neck cancer, leukemia and lymphoma; and
f. said anti-PD-1/PD-L1 immunotherapy is selected from Pembrolizumab, Nivolumab, Durvalumab and Atezolizumab.

11. (canceled)

12. (canceled)

13. (canceled)

14. (canceled)

15. (canceled)

16. The method of claim 1, wherein said combining comprises determining the total number of factors with a resistance score above a predetermined threshold and producing a total resistance score proportional to said total number.

17. The method of claim 1, further comprising performing a dimensionality reduction step with respect to said plurality of factors, to reduce the number of factors in said plurality.

18. (canceled)

19. (canceled)

20. The method of claim 18, wherein said cancer is non-small cell lung cancer (NSCLC).

21. The method of claim 1, wherein said cancer is a tyrosine kinase inhibitor resistant cancer.

22. The method of claim 1, wherein at least one of:
a. said predetermined threshold is determined by performing a cross-validation within said training set or is the median score of said training set;
b. said plurality of factors is at least 200 factors;
c. said factors expression levels are factors expression levels in a biological sample provided by said subjects;
d. said factors expression levels are factors expression levels in a biological sample selected from blood plasma, whole blood, blood serum or peripheral blood mononuclear cells provided by said subjects;
e. predicting response comprises predicting overall survival; and
f. predicting response comprises predicting progression free survival, optionally wherein progression free survival is survival at 1 year after initiation of said therapy.

23. (canceled)

24. (canceled)

25. (canceled)

26. (canceled)

27. The method of claim 1, further comprising administering said therapy to said subject predicted to respond to said therapy or administering a combined therapy comprising said anti-PD-1/PD-L1 immunotherapy and chemotherapy to said subject predicted to not respond to said therapy.

28. The method of claim 4, further comprising administering said combination therapy to said subject predicted to respond to said combination therapy or administering an alternative therapy to said subject predicted to not respond to said combination therapy.

29. The method of claim 7, further comprising administering said anti-PD-1/PD-L1 immunotherapy to said subject predicted to respond to said anti-PD-1/PD-L1 immunotherapy or administering an alternative therapy to said subject predicted to not respond to said anti-PD-1/PD-L1 immunotherapy.

30. The method of claim 1, wherein said anti-PD-1/PD-L1 immunotherapy is selected from Pembrolizumab, Nivolumab, Durvalumab and Atezolizumab.

31. The method of claim 1, wherein said chemotherapy is selected from Carboplatin, Paclitaxel, Nab-Paclitaxel, Pemetrexed, Vinorelbine, and Cisplatin.

32. The method of claim 31, wherein said combination therapy is selected from:
a. Carboplatin, Durvalumab, and Paclitaxel;
b. Atezolizumab, Bevacizumab, Carboplatin, and Paclitaxel;
c. Carboplatin, Nab-Paclitaxel, and Pembrolizumab;
d. Carboplatin, Nivolumab, and Paclitaxel;
e. Carboplatin, Nivolumab, Pemetrexed;
f. Carboplatin, Paclitaxel, Pembrolizumab;
g. Carboplatin, Paclitaxel, Pembrolizumab, and radiation;
h. Carboplatin, and Pembrolizumab;
i. Carboplatin, Pembrolizumab, and Pemetrexed;
j. Carboplatin, Pembrolizumab, and Vinorelbine; and
k. Cisplatin, Pembrolizumab, and Pemetrexed.

33. (canceled)

34. (canceled)

35. (canceled)

36. The method of claim 1, wherein
a. the subject suffers from a negative PD-L1 cancer;
b. PD-L1 high cancer comprises at least 50% of cancer cells being positive for surface expression of PD-L1 and PD-L1 low or negative cancer comprises fewer than 50% of cancer cells being positive for surface expression of PD-L1; or
c. said PD-L1 low or negative cancer is PD-L1 negative cancer comprising less than 1% of cells being positive for surface expression of PD-L1.

37. (canceled)

38. (canceled)

39. The method of claim 1, wherein said trained machine learning algorithm is trained by a method comprising:
at a training stage, training a machine learning algorithm on a training set comprising:
(i) factor expression levels of resistance-associated factors in samples from subjects suffering from cancer and known to be responsive to an anti-PD-1/PD-L1 immunotherapy and factor expression levels of resistance-associated factors in samples from subjects suffering from said cancer and known to be non-responsive to said anti-PD-1/PD-L1 immunotherapy;
(ii) at least one clinical parameter of said subjects known to be responsive and said subjects known to be non-responsive; and
(iii) labels associated with the responsiveness of said subjects suffering from said cancer;
to produce a trained machine learning algorithm, wherein said trained machine learning algorithm is trained to output said resistance score.

40. The method of claim 39, wherein said expression levels of resistance-associated factors and said at least one clinical parameter are labeled with said labels; said total resistance score predetermined threshold is 5 and a resistance score above 5 indicates the subject is resistant to the therapy or said total resistance score is converted to a total response score by the equation (10-total resistance score) and wherein a total response score above a predetermined threshold indicates the subject is responsive to therapy, optionally wherein said total response score predetermined threshold is 5; or both.

41. (canceled)
